(19) United States
(12) Patent Application Publication
(10) Pub. No.: US 2016/0159954 A1
(43) Pub. Date: Jun. 9, 2016

(54) POLYDENDRONS

(71) Applicant: The Univeristy of Liverpool

(72) Inventors: Steven Rannard, Liverpool (GB); Andrew Owen, Liverpool (GB); Hannah Rogers, Liverpool (GB); Fiona Hatton, Liverpool (GB); Faye Southworth, Liverpool (GB)

(73) Assignees: The Univeristy of Liverpool, Liverpool (GB); The Univeristy of Liverpool, Liverpool (GB)

(21) Appl. No.: 14/897,890

(22) PCT Filed: Jun. 13, 2014
(86) PCT No.: PCT/GB2014/051823

§ 371 Date: Dec. 11, 2015

(30) Foreign Application Priority Data
Jun. 14, 2013 (GB) 1310652.1

(51) Int. Cl. C08F 220/28; C08F 293/00; A61K 9/51; C08F 220/34

(52) U.S. Cl. CPC C08F 220/28; C08F 220/34; C08F 293/005; A61K 9/5138; C08F 2220/281; C08F 2220/286

(57) ABSTRACT

A method of preparing a pH-responsive non-gelled branched vinyl polymer scaffold carrying dendrons, comprising the living or controlled polymerization of a mono functional vinyl monomer and a difunctional vinyl monomer, using a dendron initiator.

The present invention relates to nanomaterials, in particular nanomaterials having hybrid structures comprising a branched vinyl polymer scaffold together with dendritic components. The present invention is particularly, though not exclusively, concerned with such hybrid materials from the perspective of medical applications, for example the carrying and delivering of drugs and other medically useful materials, the enhancement of therapeutic and diagnostic properties, and improved or more efficient or cost-effective formulations.

Dendrimers have been extensively studied in this context, amongst many other contexts. The word “dendrimer” was coined in the early 1980s, following work on cascade chemistry and arborols, to describe polymers which contain dendrons. A “dendron” is a tree-like, repeatedly-branched, moiety. Thus, a dendron is a wedge-shaped dendritic fragment of a dendrimer. Typically, dendrimers have ordered, symmetrical architectures. A dendrimer comprises a core from which several dendrons branch outwards, to form a three-dimensional, usually spherical structure.

Dendrimers can be prepared by step-wise divergent or convergent growth. Divergent procedures start at the core of the dendrimer and grow outwards. Convergent procedures prepare dendrons first and then couple the dendrons together. In convergent procedures, the dendrons are typically coupled together at their focal points (i.e. at the base of the “tree”, or the apex of the dendritic wedge) via chemically addressable groups.

For a nanomaterial to carry and deliver a drug or other biologically useful material, it is necessary for it to exhibit suitable properties in aqueous media and to have suitable domains to encapsulate the drug (which, for most drugs, need to be hydrophobic domains) and/or means of conjugating, bonding or otherwise associating with the drug. It is also advantageous for the nanomaterial to be able to carry a high “payload” of drug. Dendrimers satisfy these requirements. Due to their repeatedly branched iterative nature, they are large compared to non-polymeric active molecules and contain a large number of surface groups, and can therefore encapsulate, and/or be conjugated to, a large amount of material. Whilst they can be made from all kinds of chemical building blocks, they commonly comprise organic chains which provide hydrophobic microenvironments for drugs or other organic molecules. At the same time they can be stable in aqueous media so that drugs or other hydrophobic materials can be delivered within the body.

Whilst dendrimers have many interesting properties and promising features, they also have significant disadvantages. Dendrimer syntheses are lengthy and costly. The production of ideally branched structures requires multiple repeated steps of synthesis, purification and characterisation. Maintaining a 100% degree of branching generates complexity and takes time and requires very controlled reaction conditions. Even with high levels of successful recovery between steps, the compound effect after several steps means that the overall mass recovery suffers significantly. Whilst convergent methods are better than divergent methods from the viewpoint of ease and speed of procedure, they are still arduous, and other problems beset convergent methods, for example steric difficulties hindering coupling.

Geometric realities of iterative branching mean that the crowding constraints at the surface of the dendrimer sphere limit the size of the nanomaterials. Therefore dendrimers typically have a maximum size of about 10 nm. This limits the amount of material they can carry.

Further description of dendrimers and their structures, preparation and applications, can be found in numerous articles including: S. M. Grayson and J. M. Fréchet, Chem. Rev. 2001, 101, 3819-3867; H. Frauenrath, Prog. Polym. Sci 2005, 325-384; F. Aulenta, W. Hayes and S. Rannard, European Polymer Journal 2003, 39, 1741-1771; E. R. Gillies and J. M. J. Fréchet, Drug Discovery Today, 2005, 10, 1, 35-43; and S. H. Medina and M. E. H. El-Sayed, Chem. Rev. 2009, 109, 3141-3157.

From a first aspect the present invention provides a method of preparing a pH responsive product, said product being a non-gelled branched vinyl polymer scaffold carrying dendrons, comprising the living or controlled polymerization of a monofunctional vinyl monomer and a difunctional vinyl monomer, using a dendron initiator.

At least one dendron initiator is used in the present invention; optionally further initiators (selected from non-dendron initiators and/or other dendron initiators) may also be used in combination with the dendron initiator.

From a second aspect the present invention provides a pH responsive non-gelled branched vinyl polymer scaffold carrying a dendron moiety.

The vinyl polymer scaffold carries at least one type of dendron, and may optionally carry further moieties (selected from non-dendrons and/or other dendrons).

By “pH responsive” is meant that physical and/or chemical characteristics of the material change under different pH conditions. Such change encompasses for example: change in aggregation or solubility of the material or particles or aggregates thereof; change in stability of particles or nanoparticles of the material; change in ability to undergo association or disassociation in particular environments, e.g. aqueous environments; change in solubility, hydrophilicity or hydrophobicity which allows an active molecule, for example a drug or other payload to be carried to a particular site, for example in the body, and then released under particular pH conditions; change which allows the release of carried or encapsulated matter to be triggered or controlled; and/or cleavage of one or more bonds, e.g. resulting in breakdown of the material and/or release of payload.

The present invention provides products which can be referred to as “polydendrons” because they contain a plurality of dendrons. The dendrons may be the same or different. Polydendrons retain the advantages of dendrimers without having their disadvantages of cost, complexity and arduous synthesis. Instead of the dendritic structure extending all the way to the centre, the core is a tunable and cost-effective non-gelled branched vinyl polymer scaffold. The polydendrons typically take the form of units (which optionally are approximately spherical) with a large number of external surface dendron groups and with the vinyl scaffolds typically being present predominantly in the centre of the units.

Either the polymer scaffold or the dendrons or both may have the required pH responsive character.

One class of pH responsive polydendrons are those comprising functionality (e.g. amine or acidic functionality) within the polymer which can be protonated or deprotonated (particularly within pH ranges to be found in living systems) thereby exhibiting altered solubility. As exemplified herein, amine-containing components (e.g. amine containing meth(acrylic) polymers) can be incorporated into the polydendrons, and the amines can be protonated in acidic conditions to result in a carrier which is more hydrophilic and more soluble in aqueous systems under low pH conditions. This means that a material can be encapsulated within a polydendron nanoprecipitate, and then can be released in an acidic environment. Analogously, acid-containing components can be incorporated into polydendrons and exhibit the converse effect.

A second class of pH responsive polydendrons are those comprising functionality (e.g. amine or acidic functionality) within, or at the surface of, the dendron which can be protonated or deprotonated as described above (particularly within pH ranges to be found in living systems) thereby exhibiting altered solubility. As exemplified herein, amine-containing dendrons (e.g. tertiary amines) can be incorporated into the polydendrons, and the amines can be protonated in acidic conditions to result in a carrier with altered solubility in aqueous systems under low pH conditions. This means that a polydendron may be assembled and triggered to disassemble and material can be encapsulated and then released in an acidic environment. Analogously, acid-containing components can be incorporated into polydendrons and exhibit the converse effect.

The non-gelled branched vinyl polymer scaffolds of the present invention exhibit good solubility and low viscosity. They can be contrasted with polymer structures which are insoluble and/or exhibit high viscosity, such as extensively crosslinked insoluble polymer networks, high molecular weight linear polymers, or microgels.

The products can be made by, but are not limited to being made by, living polymerization, controlled polymerization or chain-growth polymerization. Several types of living and controlled polymerization are known in the art and suitable for use in the present invention. A preferred type of living polymerization is Atom Transfer Radical Polymerization (ATRP), however other techniques such as Reversible Addition-Fragmentation chain-Transfer (RAFT) and Nitroxide Mediated Polymerisation (NMP) or conventional free-radical polymerization controlled by the deliberate addition of chain-transfer agents are also suitable syntheses.

The skilled person is aware of techniques to provide branched but non-gelled vinyl polymer scaffolds. For example, suitable procedures are described in WO 2009/122220; N. O'Brien, A. McKee, D. C. Sherrington, A. T. Slark and A. Titterton, Polymer 2000, 41, 6027-6031; T. He, D. J. Adams, M. F. Butler, C. T. Yeoh, A. I. Cooper and S. P. Rannard, Angem. Chem. Int Ed. 2007, 46, 9243-9247; V. Bütün, I. Bannister, N. C. Billingham, D. C. Sherrington and S. P. Armes, Macromolecules 2005, 38, 4977-4982; I. Bannister, N. C. Billingham, S. P. Armes, S. P. Rannard and P. Findlay, Macromolecules 2006, 39, 7483-7492; and R. A. Slater, T. O McDonald, D. J. Adams, E. R. Draper, J. V. M. Weaver and S. P. Rannard, Soft Master 2012, 8, 9816-9827. The non-gelled and soluble products of the present invention are different to materials disclosed in L. A. Connal, R. Vestberg, C J. Hawker and G. G. Qiao, Macromolecules 2007, 40, 7855-7863 which comprise multiple cross-linking in a gelled network.

The polymerization of each vinyl polymer chain starts at an initiator. Polymerization of monofunctional vinyl monomers leads to linear polymer chains.

Copolymerization with difunctional vinyl monomers leads to branching between the chains. In order to control branching and prevent gelation there should be less than one effective brancher (difunctional vinyl monomer) per chain. Under certain conditions, this can be achieved by using a molar ratio of brancher to initiator of less than one:this assumes that the monomer (i.e. the monofunctional vinyl monomer) and the brancher (i.e. the difunctional vinyl monomer) have the same reactivity, that there is no intramolecular reaction, that the two functionalities of the brancher have the same reactivity, and that reactivity remains the same even after part-reaction. Of course, the systems and conditions may be different, but the skilled person understands how to control the reaction and determine without undue experimentation how a non-gelled structure may be achieved. For example, under dilute conditions some branchers form intramolecular cycles which limit the number of branchers that branch between chains even if the molar ratio of brancher to initiator (i.e. polymer chain) is higher than 1:1 in the reaction.

In the present invention, dendrons are used as macromolecular initiators. In order to be able to initiate polymerization, the dendrons must bear suitable reactive functionality. For example, in ATRP, convenient and effective initiators include alkyl halides (e.g. alkyl bromides), and so dendrons which carry halides at their focal points can act as initiators. In this scenario, propagation starts at the apex of the dendron “wedge”. The skilled person is well aware of the types of components and reagents which are used in ATRP and other living or controlled polymerizations, and hence the type of functionality which must be present on or introduced to dendrons for them to act as initiators.

One possible way of introducing bromo groups to dendrons is to functionalize dendron alcohols with alpha-bromoisobutyryl bromide. There are however many other ways of functionalizing dendrons so that they can act as initiators and other types of functionality which will initiate polymerization. The concept of a dendron initiator is applicable to all suitable types of polymerization and the functionality can be varied as necessary.

There is no particular limitation regarding the type of dendron that can be used, or the chemistry used to prepare the dendrons. In some scenarios it is desirable to have particular groups present at the surface (i.e. at the tips of the “branches” of the dendron), and these may be incorporated during the synthesis of the dendron. The dendrons are preferably non-vinyl.

Any suitable coupling chemistry may be used to build up the dendrons. In one example, amines and alcohols may be coupled together, for example using carbonyldiimidazole. This is, however, merely one example and numerous other coupling methods are possible.

If exclusively one type of dendron initiator is used then in the resultant hybrid branched product one end of each vinyl polymer chain bears that dendron.

Optionally, mixed initiators are used, in other words not only a dendron initiator but also at least one further initiator (which may be a different type of dendron initiator, or alternatively an initiator other than a dendron initiator) may be used. This allows considerable advantages in terms of varying the composition and the properties of the resultant polydendron structure.

The pH responsiveness may reside in one or more component of the polydendron. Either the vinyl polymer scaffold or the dendron or both [and/or other component(s) e.g. other initiator or substituent] can be tailored so as to be pH responsive. The experimental details below show the synthesis of various different polydendrons and components thereof some of which bring about pH responsive character, such that a polydendron may comprise one or more component which brings about pH responsive character and one of more component which is not.

Amine functionality has been found to be particularly useful in practice, in order to provide tunable, pH-dependent response. This is due in part to the use of a decreasing pH within various cells, tissues and organs of the body to achieve beneficial action (e.g. degradation of exogenous materials), leading to opportunities to trigger polydendron behaviour (e.g. releasing of payload drugs).

The pH responsiveness can also be linked to the hydrophilicity/hydrophobicity of the dendron or the core. The non-gelled scaffold core represents a large volume of material within the confines of the polydendron. When hydrophobic, the scaffold may provide optimal conditions for encapsulation of hydrophobic active ingredient molecules (e.g. drug molecules). As such, the polydendron offers the ability to deliver such materials in hydrophilic solvent environments. Through the response to pH, the scaffold will become hydrophilic and result in the exclusion of the encapsulated hydrophobic molecules. Polydendrons may be nanoprecipitated to form aggregated structures. If the dendron is pH-responsive, and connected to a non-responsive scaffold core, the aggregated materials will be triggered to disassemble on modification of the pH, also leading to release of encapsulated material and/or a dramatic change in the physical size of the aggregate. The pH responsive functionality of the polydendron may therefore be present exclusively at the dendron component of the polydendron, exclusively at the scaffold core of the polydendron or carried by both the core and the dendron of the polydendron.

As shown below the methodology can be tailored to make polydendrons of each of the following four types:
Hydrophilic dendron/hydrophobic core
Hydrophobic dendron/hydrophilic core
Hydrophilic dendron/hydrophilic core
Hydrophobic dendron/hydrophobic core

Each of these four types have the capacity to be pH responsive.

A further way to provide pH responsiveness is to use linkers, moieties or substituents which are cleavable under particular pH conditions, e.g. in acid environments.

The pH responsiveness is particularly advantageous for drug delivery, drug transport and drug release applications. It also has applications when material or payload other than drug is carried.

Thus, for example, pH sensitive cores can be used which can release their drug or other material when the polydendron material enters a highly acidic cellular compartment.

In contrast conventional dendrimer polymers have internal chemistry which is redundant and merely serves as a scaffold.

Multiple modes of responsivity can be used, e.g. a triple trigger release mechanism can be based on the use of a pH responsive polymer and acid cleavable linker so that a drug may be released and the polydendron aggregate may break down and further degrade to low molecular weight components that more easily leave the body.

Thus the present invention provides significant benefits in terms of improved drug delivery methods, improved drug release, targeting, administration, dose and breakdown of drug carriers to facilitate their removal from the body. Drugs that may be encapsulated include, but are not limited to, hydrophobic agents for chronic and acute, oral, topical, opthalmic and parenteral administration of anticancer, infectious disease, age-related disease, genetic, CNS, psychiatric, paediatric and parasitic therapies. Some non-limiting examples of functional groups which may bring about pH responsiveness include the following which may be present on the dendron(s), within the core, or both: amines (e.g. primary, secondary or tertiary amines, cyclic aliphatic and cyclic aromatic amine moieties); carboxylic acids including aromatic and aliphatic acids; non-carboxylic acids such as sulphonic acids and organic sulphate groups; betaines, acetals, ketals, t-butoxy carbonyl groups, acetates and acetoxy functionality.

Optionally, the drug or other payload is released at a pH of about 5 to about 8.5, e.g. about 5.5 to 8, or 6 to 7.5.

The present invention resides in the combination of features which work well together. The branched vinyl polymer methodology is intermingled with the use of at least one dendron initiator. The way in which the living or controlled polymerization occurs means that, if different initiators are used, these will be distributed statistically and evenly around the surface of the non-gelled branched vinyl polymer scaffold. Some polymer chains will have one type of initiator at one end whereas other polymer chains will have another type at their end. There may be one type of initiator, two types of initiator, or more, e.g. three or four or more, and therefore the multiplicity of types of end group may be one or more.

The vinyl polymer core is easily tunable and very cost-effective. Different types of monomers, with different properties (e.g. differing solubility properties) may be used. The methodology allows a sizeable scaffold to be built, and the molecular weight and size can be controlled by choice of particular monomers (a wide range can be used) and reaction conditions, for example the ratio of initiator to monomer.

The material is non-gelled and therefore soluble. At the same time the option to use different types of initiator, or mixed initiators, allows further tunability and flexibility. There are synergistic advantages: for example the use of dendrons and other moieties as initiators means that they do not need to be introduced separately but instead are used as reagents within an already very efficient and convenient polymerization process, The process conveniently and cost-effectively results in the the initiators being distributed throughout the materials. The initiators themselves are relatively easy to synthesize. Regarding the need for the initiators to have suitable means and functionality to initiate polymerization, the considerations described above in relation to the dendron initiators apply mutatis mutandis to any further initiator(s) which may be used.

The living or controlled polymerization methodology inherently allows control in the synthesis of the polymeric scaffold. For example, ATRP and other techniques are robust and flexible in being suitable for use with a large variety of functional groups and in avoiding unwanted side reactions. The size and dispersity of the products can be controlled. The monomer units are usually homogeneously distributed between the initiator molecules and therefore the chain length, and hence the molecular weight, can be controlled. The conditions can be controlled to result in materials having low polydispersity indexes when forming linear polymers, i.e. mixtures wherein the individual components have approximately the same size. This is particularly useful in the present invention as the individual chains comprising the branched structure (i.e. the primary chains) have similar chain lengths. The resulting branched polymers of the invention have a distribution of structures with varying numbers of linear chains connected to form the branched architectures.

The optional use of at least one further initiator, in addition to the dendron initiator, within the living or controlled polymerization methodology, brings further advantages. The further initiator alters the properties of the polydendron, for example the solubility, hydrophilicity, hydrophobicity, aggregation, size, reactivity, stability, degradability, therapeutic, diagnostic, biological transport, plasma residence time, cell interaction, drug compatibility, stimulus response, targeting and/or imaging characteristics.

The optional further initiator may comprise or be derived from one or more of the following: a small molecule, a drug, an active pharmaceutical ingredient, a polymer, a peptide, a sugar, a dendron, a moiety which carries or can carry a drug, an anionic functional group, a cationic functional group, a moiety which enhances solubility (for example, of the polydendron within aqueous systems, or of a drug or other carried material), a moiety which prolongs residence time within the body, a moiety which enhances stability of a drug or other active material, a moiety which reduces macrophage uptake, a moiety which enhances controlled release, a moiety which enhances drug transport, or a moiety which enhances drug targeting.

The initiator may be a macroinitiator, for example a macroinitiator prepared by synthesis from one or more monomer (e.g. a water soluble monofunctional monomer), or a macroinitiator prepared by modification of a pre-synthesized polymer. The macroinitiator may be a copolymer, i.e. may comprise a polymer made from at least two monomers, e.g. monofunctional monomers. The macroinitiator may further be selected from natural polymers, for example water soluble or partially soluble polymers, e.g. polysaccharides, polypeptides or proteins.

Each type of initiator may fall within one or more than one of the above definitions; for example the initiator may be a dendron and may also carry a drug. The initiator may also be a pro-drug, releasing a moiety that becomes pharmacologically active after a further process within the body.

The present inventors have been surprised at how effective the present invention is, in allowing a range of properties to be controlled and tuned. As described in more detail below, they have observed: that the surface chemistry can be varied widely across a hydrophobic—amphiphilic—hydrophilic spectrum; that the encapsulation environment can be varied significantly; that the salt stability can be controlled; and that transcellular permeability (in an in vitro model) can be tuned and improved.

In view of the drug delivery capabilities, from further aspects the present invention also provides pharmaceutical compositions comprising the products of the present invention, and allows enhancements in terms of medical administration possibilities.

For example, the surprisingly effective way in which the polydendrons interact controllably with, and transport encapsulated materials through, model gut-epithelium, is relevant to oral delivery applications. Materials of this type are also useful within parenteral administration such as intravenous, subcutaneous and intramuscular injection.

Polyethylene glycol (PEG) groups are advantageous for use in the initiators of the present invention. In comparison to polydendrons which carry dendrons alone, polydendrons which carry not only dendrons but also PEG groups exhibit enhanced stability in aqueous systems, controlled interaction with cells, and prolonged systemic half-life. Non-limiting examples of suitable PEGs include those with end functionality such as methyl, hydroxyl, amine, acid etc, functionality, and/or those with molecular weights above 300 g/mol, preferably those with hydroxyl and acid functional chains and/or with molecular weights >750 g/mol. Particularly preferred are hydroxyl compounds and/or those with molecular weights >1000 g/mol. Alternatively, other chemical moieties which function in the same or similar way and which can advantageously be used in the present invention include acrylate and methacrylate moieties including water-soluble polymeric chains (e.g. less than 20000 g/mol), for example derived from vinyl or non vinyl monomers such as ethylene glycol methacylate, glycerol methacrylate, vinyl alcohol, acrylic acid, methacrylic acid, or hydroxyethyl methacrylate.

The initiators may include groups which allow post-functionalization of the polydendrons. Thus, whilst various possible initiator structures and moieties have been discussed above, an alternative to them being present within the initiator at the start of the reaction is to incorporate them later by reaction of the polydendron with suitable materials.

Suitable functional groups in initiators which allow post-functionalization include thiols, hydroxyl groups, amines, acids or isocyanates, amongst others.

For example, N-hydroxysuccinimide functionalized initiators can be incorporated into polydendrons and post-functionalized with materials containing amine groups.

The several means of flexibility and levels of control provided by the present invention reside in the ability to alter several variables including: the amount of initiator(s) relative to vinyl polymer, the ratio between dendron initiator(s) and non-dendron initiator(s) [or other dendron initiator(s)], the nature and properties of the dendron initiator(s), the nature and properties of non-dendron initiator(s), the extent of branching, the nature and properties of the monomer(s), the nature and properties of the brancher(s), and the capacity of the nanomaterials for drugs or other materials.

A further advantage of the methods and products of the present invention is that they are compatible with the preparation of nanomaterials which are stable and of controllable and uniformts size. Nanoprecipitation of branched vinyl polymers is disclosed in R. A. Slater, T. O McDonald, D. J. Adams, E. R. Draper, J. V. M. Weaver and S. P. Rannard, Soft Matter 2012, 8, 9816-9827. This technique has been successfully used on single and mixed initiator—carrying polydendrons of the present invention to prepare stable nanoparticles. The nanoparticles are prepared by the self assembly during precipitation with dispersity and size of these nanoparticles being effectively controlled by varying the nature of the solvents, precipitation method, concentration, and presence of other components. Uniform or near uniform assembled nanoparticle sizes with low polydispersities can be achieved. Nanoparticles of uniform and controllable size are extremely useful in the field of drug encapsulation and delivery.

The nanoparticles may for example be prepared by precipitation of the polydendron out of solution using a solvent which is a non-solvent for the vinyl polymer scaffold but which is a good solvent for the dendrons or other surface groups.

This nanoprecipitation using a solvent switch might have been expected to lead to collapse of the internal vinyl polymer core, but self-assembly of the individual polydendron particles is observed leading to very stable distributions of larger complex nanoparticles with a narrow size distribution.

A preferred “non-solvent” for the vinyl polymer, i.e. medium in which the nanoprecipitate particles are stable, is water.

By way of example, where the core is a polyHPMA-EGDMA material and the dendrons are selected from amine functional dendrons (e.g. G1A, G1D and G2D shown in the examples), then the material can be first dissolved in THF and nanoprecipitated into water.

The characteristics of the polydendron, including the electronic/charge and steric nature, and the nature of the solvent, affect the way in which the material behaves in that solvent. Without wishing to be bound by theory, the particles generally increase in size until they reach a colloidally stable state during the nanoprecipitation process.

As exemplified below, the present invention allows the encapsulation and release of not only organic materials—e.g. nile red, simulating encapsulation of a drug—but also inorganic materials—e.g. magnetic particles. This expands the utility of the present invention to cover further therapeutic and targeting uses. The encapsulation of inorganic material (e.g. magnetic material, e.g. iron oxide) in polydendrons may also be considered as a standalone invention within this disclosure.

The branches are typically distributed statistically throughout the connected linear polymer chains (rather than discretely in block polymerised monofunctional vinyl monomers and difunctional vinyl monomers). Each branch may be a glycol diester branch, for example.

The difunctional vinyl monomer acts as a brancher (or branching agent) and provides a branch between adjacent polymer chains. The branching agent may have two or more vinyl groups.

The monofunctional monomer utilised for the primary chain may comprise any carbon-carbon unsaturated compound which can be polymerised by an addition polymerisation mechanism, for example vinyl and allyl compounds. The monofunctional monomer may be hydrophilic, hydrophobic, amphiphilic, anionic, cationic, neutral or zwitterionic in nature.

The monofunctional monomer may be selected from but is not necessarily limited to monomers such as: vinyl acids and derivatives (including esters, amides and anhydrides), vinyl aryl compounds, vinyl ethers, vinyl amines and derivatives (including aryl amines), vinyl nitriles, vinyl ketones, and derivatives of the aforementioned compounds as well as corresponding allyl variants thereof

Vinyl acids and derivatives thereof include: (meth)acrylic acid, fumaric acid, maleic acid, itaconic acid and acid halides thereof such as (meth)acryloyl chloride.

Vinyl acid esters and derivatives thereof include: C1 to C20 alkyl(meth)acrylates (linear and branched) such as for example methyl (meth)acrylate, stearyl (meth)acrylate and 2-ethyl hexyl (meth)acrylate; aryl(meth)acrylates such as for example benzyl (meth)acrylate; tri(alkyloxy)silylalkyl(meth)acrylates such as trimethoxysilylpropyl(meth)acrylate; and activated esters of (meth)acrylic acid such as N-hydroxysuccinamido (meth)acrylate.

Vinyl aryl compounds and derivatives thereof include: styrene, acetoxystyrene, styrene sulfonic acid, 2- and 4-vinyl pyridine, vinyl naphthalene, vinylbenzyl chloride and vinyl benzoic acid.

Vinyl acid anhydrides and derivatives thereof include: maleic anhydride.

Vinyl amides and derivatives thereof include: (meth)acrylamide, N-(2-hydroxypropyl)methacrylamide, N-vinyl pyrrolidone, N-vinyl formamide, (meth)acrylamidopropyl trimethyl ammonium chloride, [3-((meth)acrylamido)propyl]dimethyl ammonium chloride, 3-[N-(3-(meth)acrylamidopropyl)-N,N-dimethyl]aminopropane sulfonate, methyl (meth)acrylamidoglycolate methyl ether and N-isopropyl(meth)acrylamide.

Vinyl ethers and derivatives thereof include: methyl vinyl ether.

Vinyl amines and derivatives thereof include: dimethylaminoethyl (meth)acrylate, diethylaminoethyl (meth)acrylate, diisopropylaminoethyl (meth)acrylate, mono-t-butylaminoethyl (meth)acrylate, morpholinoethyl(meth)acrylate and monomers which can be post-reacted to form amine groups, such as N-vinyl formamide. Vinyl aryl amines and derivatives thereof include: vinyl aniline, 2 and 4-vinyl pyridine, N-vinyl carbazole and vinyl imidazole.

Vinyl nitriles and derivatives thereof include: (meth)acrylonitrile. Vinyl ketones or aldehydes and derivatives thereof include: acreolin.

Monomers based on styrene or those containing an aromatic functionality such as styrene, α-methyl styrene, vinyl benzyl chloride, vinyl naphthalene, vinyl benzoic acid, N-vinyl carbazole, 2-, 3- or 4-vinyl pyridine, vinyl aniline, acetoxy styrene, styrene sulfonic acid, vinyl imidazole or derivatives thereof may also be used.

Other suitable monofunctional monomers include: hydroxyl-containing monomers and monomers which can be post-reacted to form hydroxyl groups, acid-containing or acid-functional monomers, zwitterionic monomers and quaternised amino monomers.

Hydroxyl-containing monomers include: vinyl hydroxyl monomers such as hydroxyethyl (meth)acrylate, 1- and 2-hydroxy propyl (meth)acrylate, 2-hydroxy methacrylamide, glycerol mono(meth)acrylate and sugar mono(meth)acrylates such as glucose mono(meth)acrylate.

Monomers which can be post-reacted to form hydroxyl groups include: vinyl acetate, acetoxystyrene and glycidyl (meth)acrylate.

Acid-containing or acid functional monomers include: (meth)acrylic acid, styrene sulfonic acid, vinyl phosphonic acid, vinyl benzoic acid, maleic acid, fumaric acid, itaconic acid, 2-(meth)acrylamido 2-ethyl propanesulfonic acid, mono-2-((meth)acryloyloxy)ethyl succinate and ammonium sulfatoethyl (meth)acrylate.

Zwitterionic monomers include: (meth)acryloyl oxyethylphosphoryl choline and betaines, such as [2-((meth)acryloyloxy)ethyl]dimethyl-(3-sulfopropyl)ammonium hydroxide.

Quaternised amino monomers include: (meth)acryloyloxyethyltri-(alkl/aryl)ammonium halides such as (meth)acryloyloxyethyltrimethyl ammonium chloride.

Oligomeric, polymeric and di- or multi-functionalised monomers may also be used, especially oligomeric or polymeric (meth)acrylic acid esters such as mono(alk/aryl) (meth)acrylic acid esters of polyalkyleneglycol or polydimethylsiloxane or any other mono-vinyl or allyl adduct of a low molecular weight oligomer.

Oligomeric and polymeric monomers include: oligomeric and polymeric (meth)acrylic acid esters such as mono(alk/aryl)oxypolyalkyleneglycol (meth)acrylates and mono(alk/aryl)oxypolydimethyl-siloxane(meth)acrylates. These esters include for example: monomethoxy oligo(ethyleneglycol) mono(meth)acrylate, monomethoxy oligo(propyleneglycol) mono(meth)acrylate, monohydroxy oligo(ethyleneglycol) mono(meth)acrylate, monohydroxy oligo(propyleneglycol) mono(meth)acrylate, monomethoxy poly(ethyleneglycol) mono(meth)acrylate, monomethoxy poly(propyleneglycol) mono(meth)acrylate, monohydroxy poly(ethyleneglycol) mono(meth)acrylate and monohydroxy poly(propyleneglycol) mono(meth)acrylate.

Vinyl acetate and derivatives thereof can also be utilised.

Further examples include: vinyl or allyl esters, amides or ethers of pre-formed oligomers or polymers formed via ring-opening polymerisation such as oligo(caprolactam), oligo(caprolactone), poly(caprolactam) or poly(caprolactone), or oligomers or polymers formed via a living polymerisation technique such as poly(1,4-butadiene).

The corresponding allyl monomers to those listed above can also be used where appropriate.

Specific examples of monofunctional monomers include:

amide-containing monomers such as (meth)acrylamide, N-(2-hydroxypropyl)methacrylamide, N,N′-dimethyl(meth)acrylamide, N and/or N′-di(alkyl or aryl) (meth)acrylamide, N-vinyl pyrrolidone, [3-((meth)acrylamido)propyl]trimethyl ammonium chloride, 3-(dimethylamino)propyl(meth)acrylamide, 3-[N-(3-(meth)acrylamidopropyl)-N,N-dimethyl]aminopropane sulfonate, methyl (meth)acrylamidoglycolate methyl ether and N-isopropyl(meth)acrylamide;

(meth)acrylic acid and derivatives thereof such as (meth)acrylic acid, (meth)acryloyl chloride (or any halide), (alkylaryl)(meth)acrylate;

vinyl amines such as aminoethyl (meth)acrylate, dimethylaminoethyl (meth)acrylate, diethylaminoethyl (meth)acrylate, diisopropylaminoethyl (meth)acrylate, mono-t-butylamino (meth)acrylate, morpholinoethyl(meth)acrylate; vinyl aryl amines such as vinyl aniline, vinyl pyridine, N-vinyl carbazole, vinyl imidazole, and monomers which can be post-reacted to form amine groups, such as vinyl formamide;

vinyl aryl monomers such as styrene, vinyl benzyl chloride, vinyl toluene, alpha-methyl styrene, styrene sulfonic acid, vinyl naphthalene and vinyl benzoic acid;

vinyl hydroxyl monomers such as hydroxyethyl (meth)acrylate, hydroxy propyl (meth)acrylate, glycerol mono(meth)acrylate or monomers which can be post-functionalised into hydroxyl groups such as vinyl acetate, acetoxy styrene and glycidyl (meth)acrylate;

acid-containing monomers such as (meth)acrylic acid, styrene sulfonic acid, vinyl phosphonic acid, vinyl benzoic acid, maleic acid, fumaric acid, itaconic acid, 2-(meth)acrylamido 2-ethyl propanesulfonic acid and mono-2-((meth)acryloyloxy)ethyl succinate or acid anhydrides such as maleic anhydride;

zwitterionic monomers such as (meth)acryloyl oxyethylphosphoryl choline and betaine-containing monomers, such as [2-((meth)acryloyloxy)ethyl]dimethyl-(3-sulfopropyl)ammonium hydroxide;

quaternised amino monomers such as (meth)acryloyloxyethyltrimethyl ammonium chloride.

vinyl acetate or vinyl butanoate or derivatives thereof.

The corresponding allyl monomer, where applicable, can also be used in each case. Mixtures of more than one monomer may also be used to give statistical, graft, gradient or alternating copolymers.

Some preferred monofunctional vinyl monomers include methacrylate monomers or styrene. Some preferred hydrophobic methacrylate monomers include 2-hydroxypropyl methacrylate (HPMA), n-butyl methacrylate (nBuMA), tert-butyl methacrylate (tBuMA), and oligo(ethylene glycol) methyl ether methacrylate (OEGMA). HPMA is particularly preferred, and is readily available or synthesised as a mixture of (predominantly) 2-hydroxypropyl methacrylate and 2-hydroxyisopropyl methacrylate. A preferred hydrophilic methacrylate monomers is diethylaminoethyl methacrylate (DEAEMA).

The polydendron also contains a brancher which is a multifunctional (at least difunctional) vinyl containing molecule.

The multifunctional monomer or brancher may comprise a molecule containing at least two vinyl groups which may be polymerised via addition polymerisation. The molecule may be hydrophilic, hydrophobic, amphiphilic, neutral, cationic, zwitterionic, oligomeric or polymeric. Such molecules are often known as cross-linking agents in the art.

Examples include: di- or multivinyl esters, di- or multivinyl amides, di- or multivinyl aryl compounds, di- or multivinyl alk/aryl ethers. Typically, in the case of oligomeric or polymeric di- or multifunctional branching agents, a linking reaction is used to attach a polymerisable moiety to a di- or multifunctional oligomer or polymer. The brancher may itself have more than one branching point, such as T-shaped divinylic oligomers or polymers. In some cases, more than one multifunctional monomer may be used. The corresponding allyl monomers to those listed above can also be used where appropriate.

Preferred multifunctional monomers or branchers include but are not limited to: divinyl aryl monomers such as divinyl benzene; (meth)acrylate diesters such as ethylene glycol di(meth)acrylate, propyleneglycol di(meth)acrylate and 1,3-butylenedi(meth)acrylate;

polyalkylene oxide di(meth)acrylates such as tetraethyleneglycol di(meth)acrylate, poly(ethyleneglycol) di(meth)acrylate and poly(propyleneglycol) di(meth)acrylate; divinyl (meth)acrylamides such as methylene bisacrylamide; silicone-containing divinyl esters or amides such as (meth)acryloxypropyl-terminated poly(dimethylsiloxane); divinyl ethers such as poly(ethyleneglycol)divinyl ether; and tetra- or tri-(meth)acrylate esters such as pentaerythritol tetra(meth)acrylate, trimethylolpropane tri(meth)acrylate or glucose di- to penta(meth)acrylate.

Further examples include: vinyl or allyl esters, amides or ethers of pre-formed oligomers or polymers formed via ring-opening polymerisation such as oligo(caprolactam), oligo(caprolactone), poly(caprolactam) or poly(caprolactone), or oligomers or polymers formed via a living polymerisation technique such as oligo- or poly(1,4-butadiene).

Some preferred types of difunctional vinyl monomers include dimethacrylate monomers, for example ethyleneglycol dimethacrylate (EGDMA).

The molar ratio of difunctional vinyl monomer to initiator is preferably no more than 2, more preferably no more than 1.5, and most preferably no more than 1 if conducted under appropriate conditions.

The amount of difunctional vinyl monomer relative to monofunctional vinyl monomer is preferably 7.5 mol % or less, 2 mol % or less, or 1.6 mol % or less, more preferably between 1 and 7.5 mol %, for example between 1 and 2 mol %

In a preferred embodiment, the method is a one-pot method. In this embodiment, the reaction of monofunctional vinyl monomer, difunctional vinyl monomer and initiators is carried out conveniently and cost-effectively.

Preferably the method comprises preparing a mixture of the monofunctional vinyl monomer, difunctional vinyl monomer and initiators under suitable conditions. The mixture may contain a catalyst (such as CuCl) or additional agents depending on the addition polymerisation technique being used. The mixture may also contain a ligand (such as 2,2′-bipyridine). The mixture may also contain a chain transfer agent.

Suitable ATRP initiators include isobutyrate esters, preferably haloisobutyrate esters, most preferably bromoisobutyrate esters. Thus the initiator can for example have the following general formula I:

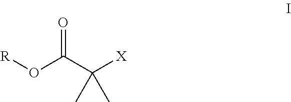

wherein X denotes a chemically addressable group and is preferably a halide, for example Cl or Br, most preferably Br, and wherein R is any suitable organic moiety. Where the initiator is a dendron initiator, R is branched into a dendritic wedge and X is the chemically addressable group at the apex of the dendritic wedge. Whilst isobutyryl esters are convenient and effective to use in this context, other chemistries are possible.

It of course will be understood that part of the initiator (in this case the X group, usually bromide) is present in the initiator but reacts during the process so that it is not necessarily present in the product at the end of all primary chains.

Where the initiator of general formula I is a dendron initiator, R is a moiety which divides into two or more (preferably two) first generation branches (preferably identical first generation branches). Optionally each of those first generation branches then divides into two or more (preferably two) second generation branches (preferably identical second generation branches). Optionally each of those second generation branches then divides into two or more (preferably two) third generation branches (preferably identical third generation branches). There may analogously be further generations of branching. A dendron having only first generation branches is known as a generation 1 dendron; a Dendron having first and second generation branches is known as a generation 2 dendron.

The outermost branches of the dendron (the part most likely to end up on the surface of the polydendron) may comprise one or more of a variety of chemical groups, for example aromatic groups (e.g. benzene rings, e.g. of benzyloxy groups), amines (e.g. tertiary amines), alkyl groups (e.g. alkyl chains or branched alkyl groups e.g. tertiary butyl groups), amide groups, xanthates or carbamates (e.g. terminating in a tertiary butyl group). These are however merely non-limiting examples: many chemistries are possible. One of the advantages of the present invention is that is compatible with a wide variety of different types of dendrons and other groups; the flexibility provided by the use of mixed initiators is considerable. The properties can be tuned by selecting dendrons with different chemical constituents and/or different surface groups, for example hydrophilic or hydrophobic groups, large or small moieties, groups of different polar or electronic character, groups which may allow further conjugation, etc.

Each segment may comprise one or more of an alkyl chain, ester, carbamate, or other linking group. Again these are merely non-limiting examples and many chemistries are possible.

Within the dendron, the structure may divide at any suitable point, for example a carbon atom or a nitrogen atom, or a larger moiety such as a ring. For example the structure may comprise a N,N-bis-substituted amino component, e.g. esters of 1-[N,N-bis-substituted amino]-2-propanol.

Some specific and non-limiting examples of possible dendrons will now be described.

A first class of possible dendrons include those having benzyloxy surface groups. For example the surface group may have the following structure:

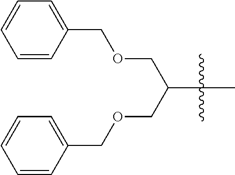 [SMILES: *C(COCc1ccccc1)COCc1ccccc1]

Optionally two of these moieties may be linked via carbamate chains to an amide branching point.

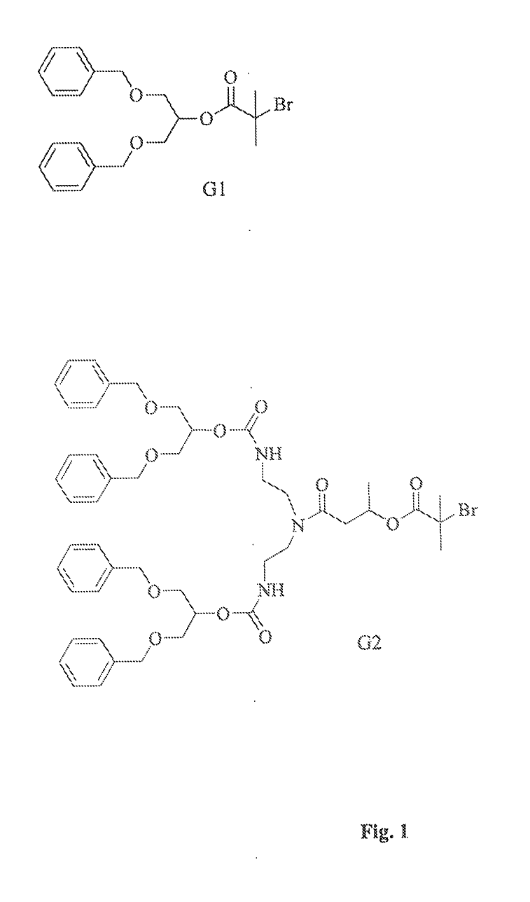

Examples in this class of dendrons include the G1 and G2 structures shown in FIG. 1.

A second class of possible dendrons include those having tertiary amine surface groups, for example where the end amines are dimethyl substituted. Optionally the branching may occur at tertiary amine centres and the segments may contain ester linkages.

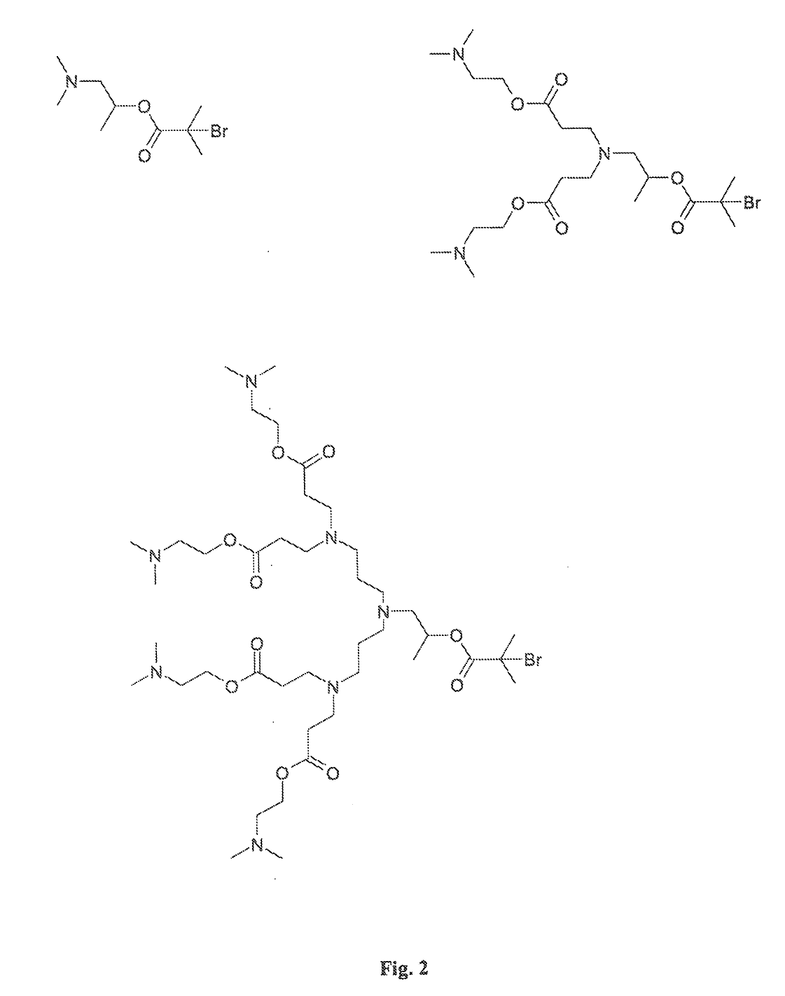

Examples in this class of dendrons, and a suitable component thereof; are shown in FIG. 2.

A third class of possible dendrons include those having carbamate surface functionality, for example tertiary butyl carbamates, and optionally carbamate functionality within the segment(s).

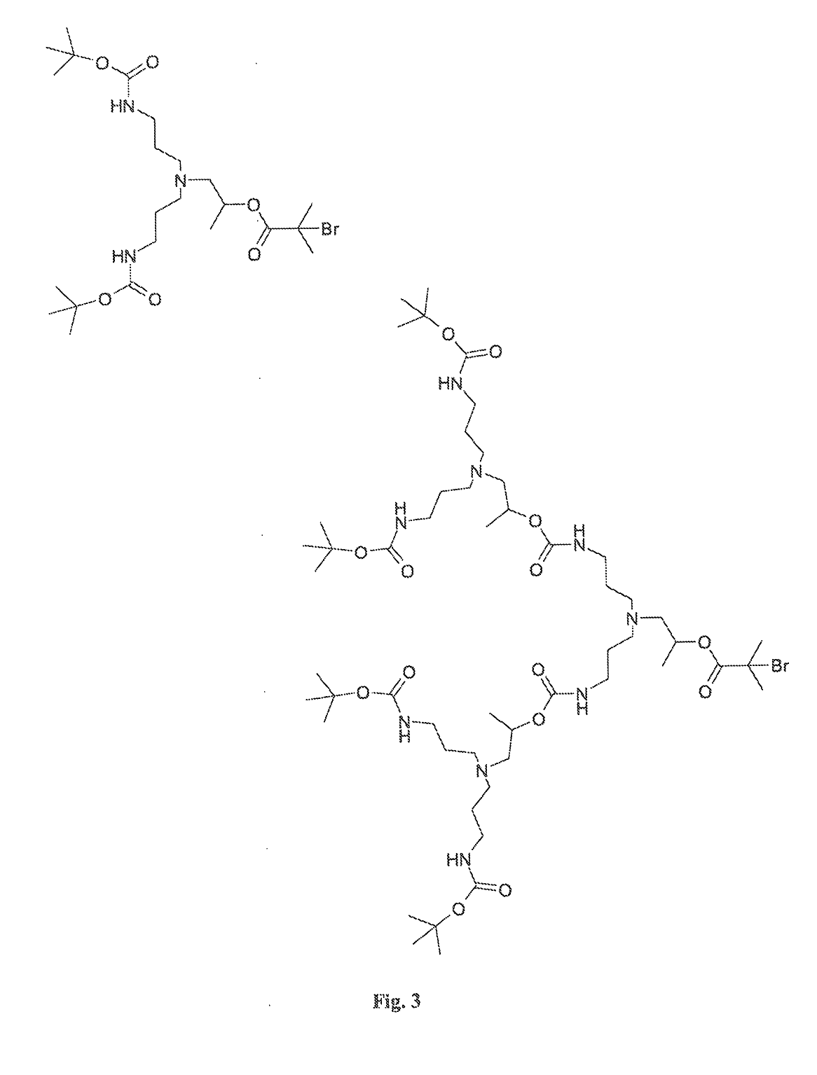

Examples in this class of dendrons are shown in FIG. 3.

A fourth class of possible dendrons include those having xanthate functionality, optionally with branches comprising esters.

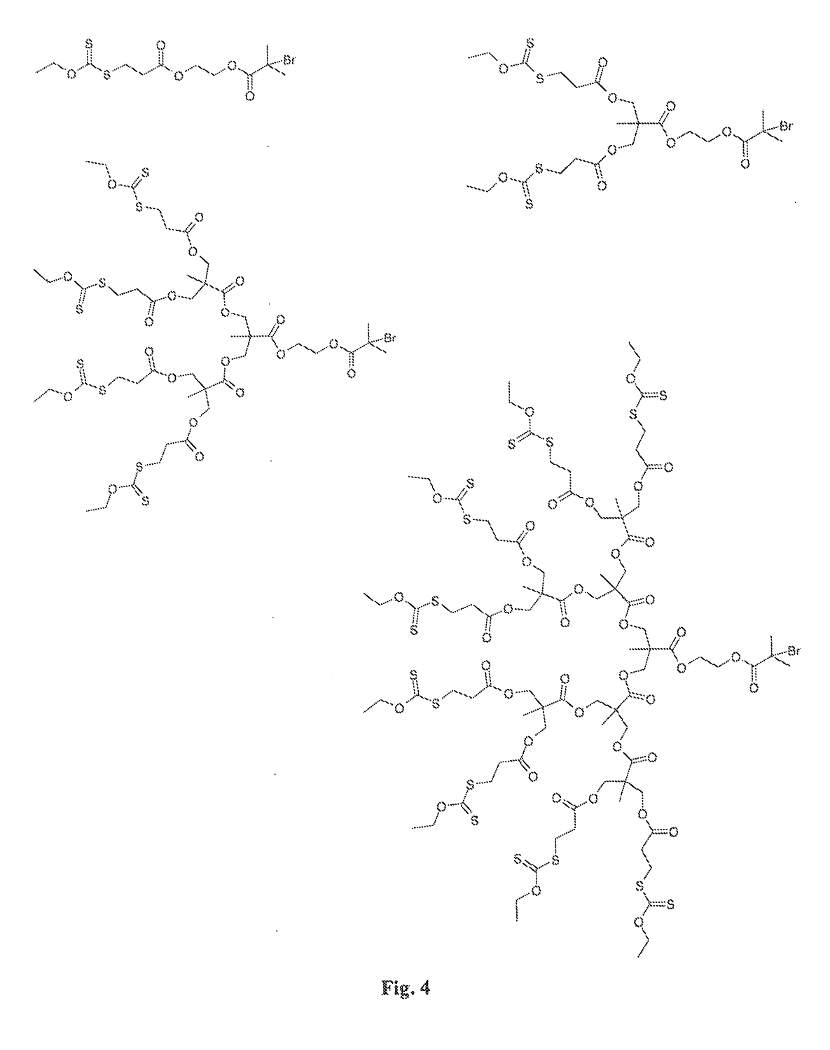

Examples in this class of dendrons, and a suitable component thereof, are shown in FIG. 4.

The dendrons may be prepared by known chemical techniques. Some possible methods of preparation include those described below.

The present invention will now be described in further non-limiting detail and with reference to the Examples and Figures in which:

FIGS. 1 to 4 show some examples of dendron initiators and components thereof which can be used in the present invention;

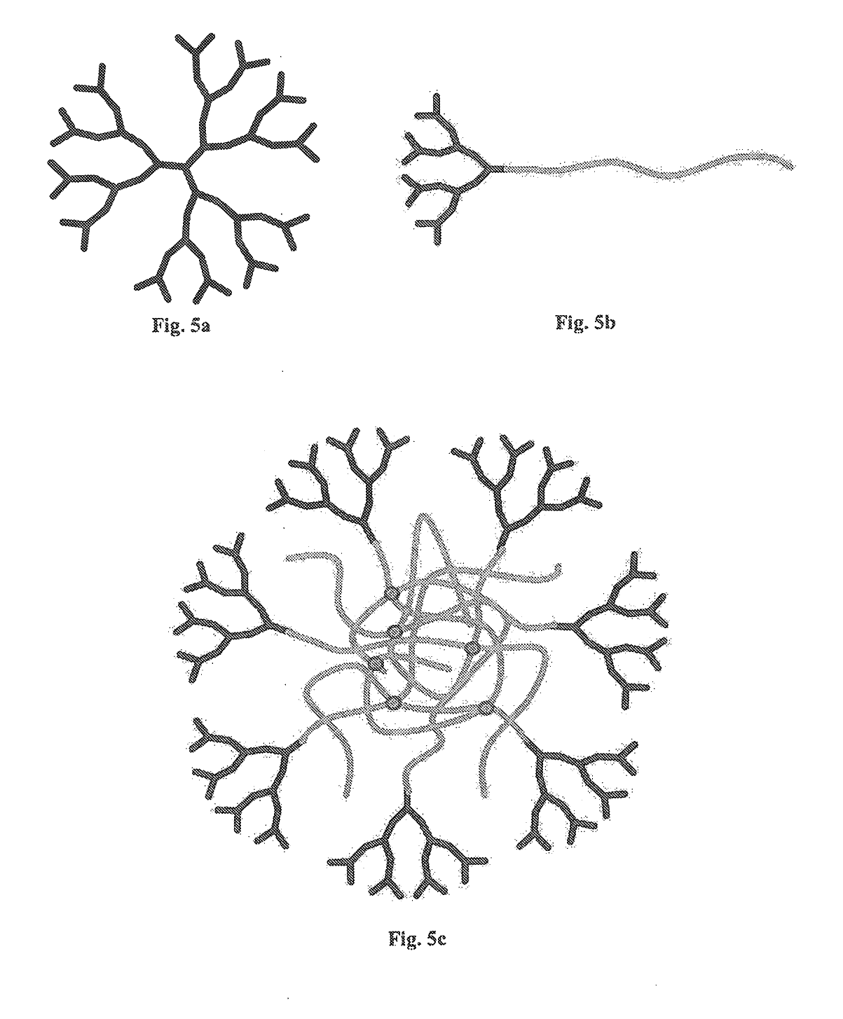

FIG. 5 shows, schematically, structural differences between dendrimers and polydendrons;

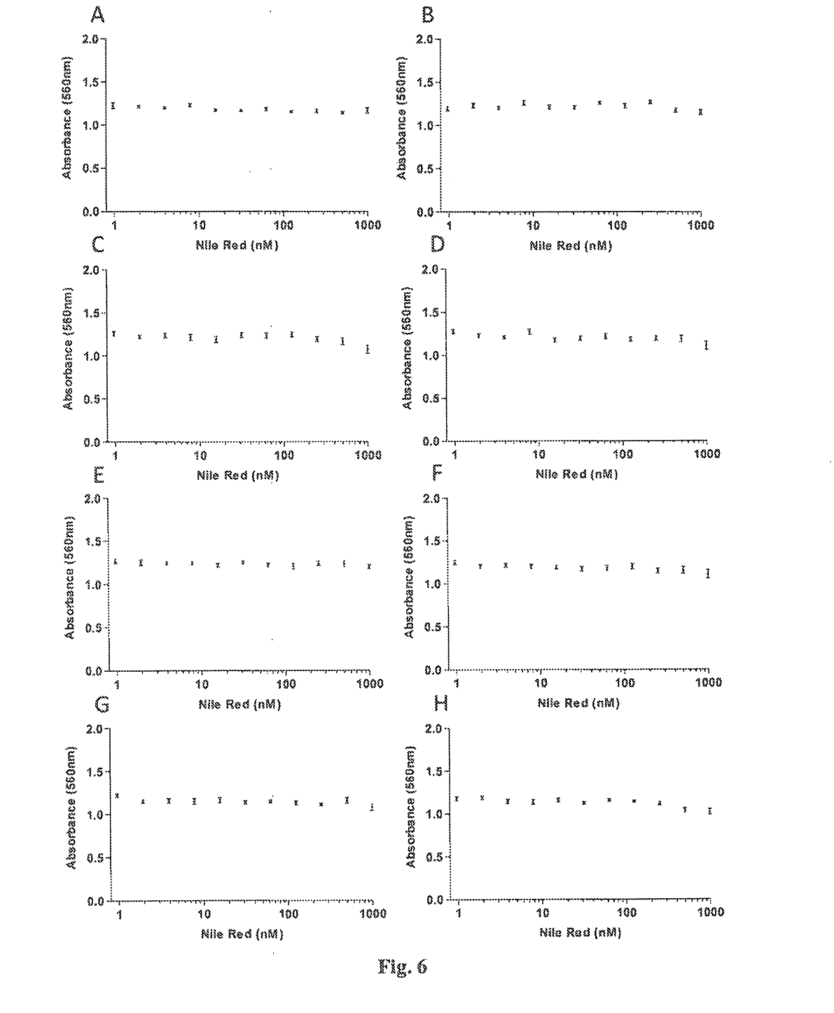

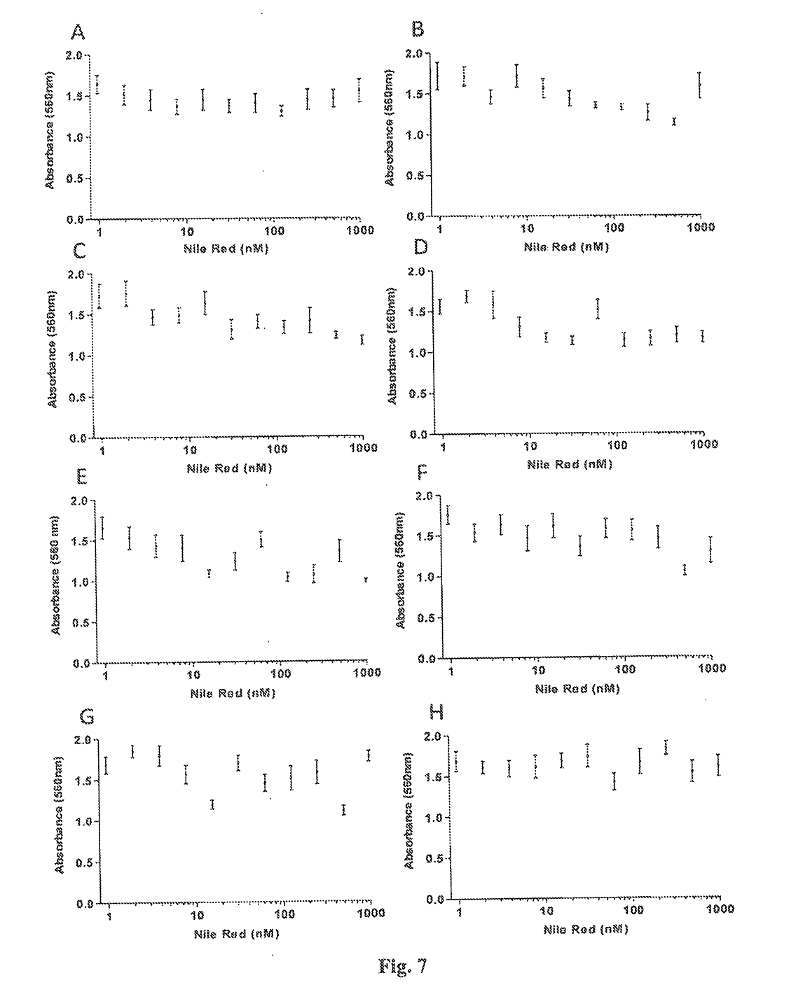

FIGS. 6 and 7 show MTT assays of Caco-2 cells following incubation with aqueous Nile Red and polydendrons;

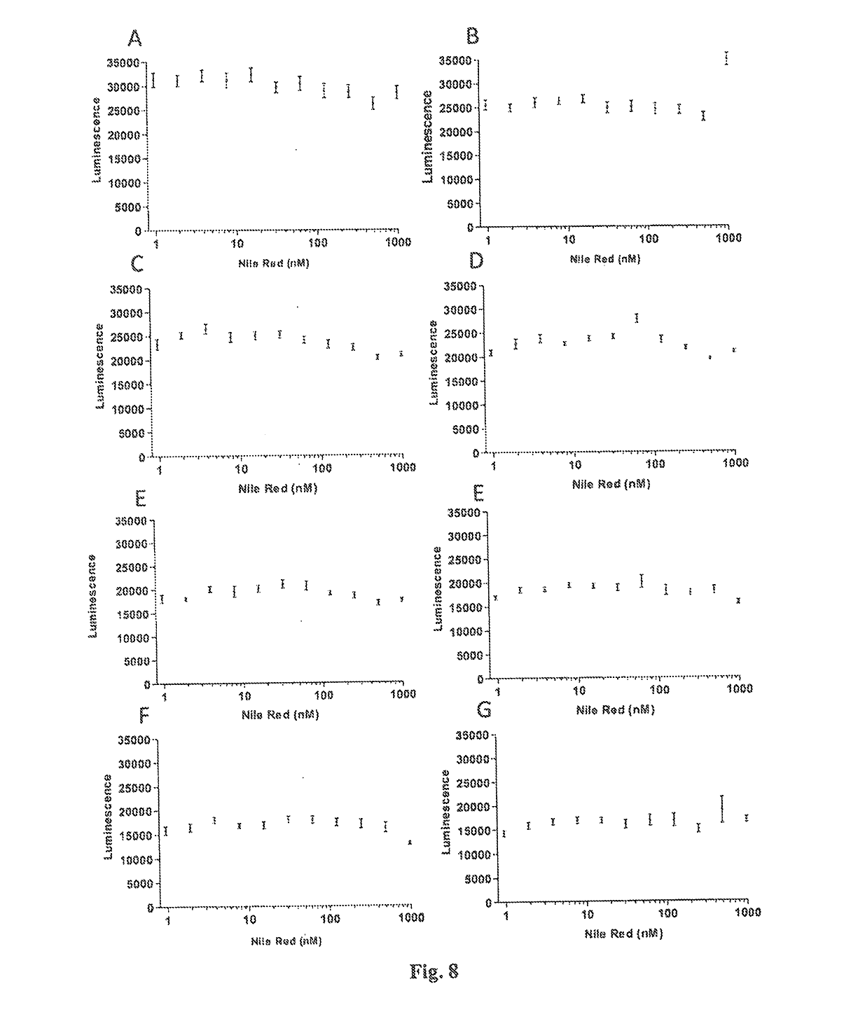

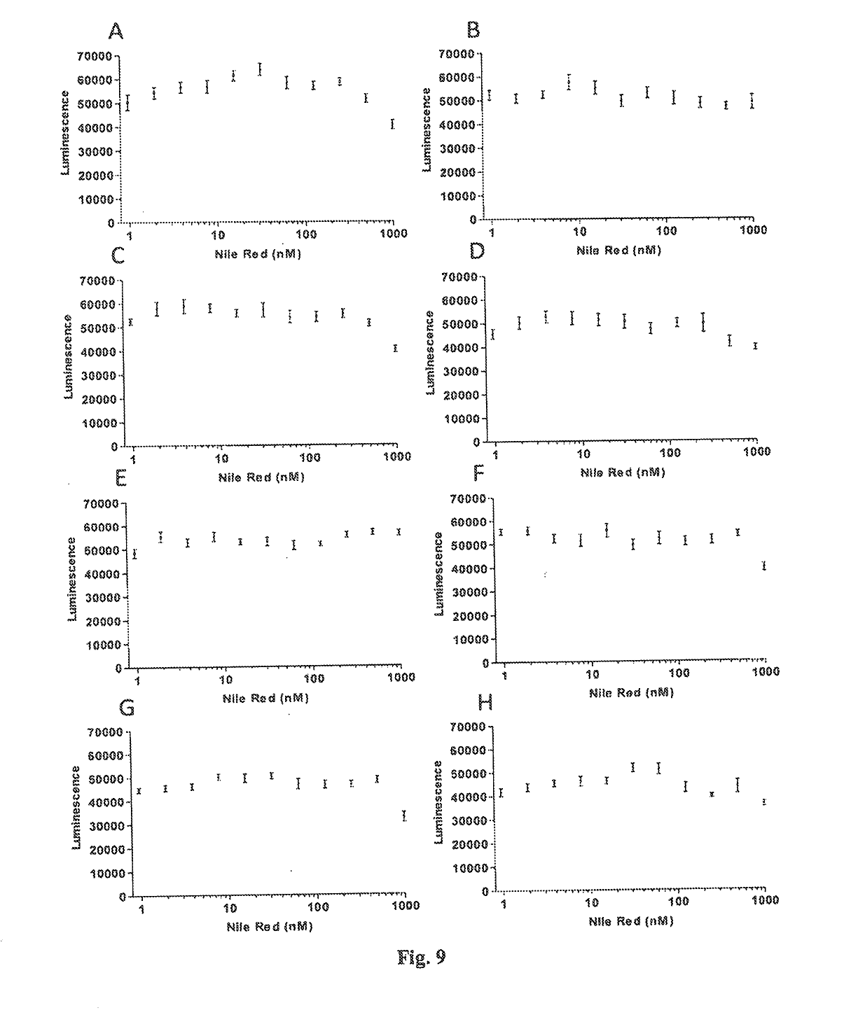

FIGS. 8 and 9 show ATP assays of Caco-2 cells following incubation with aqueous Nile Red and polydendrons;

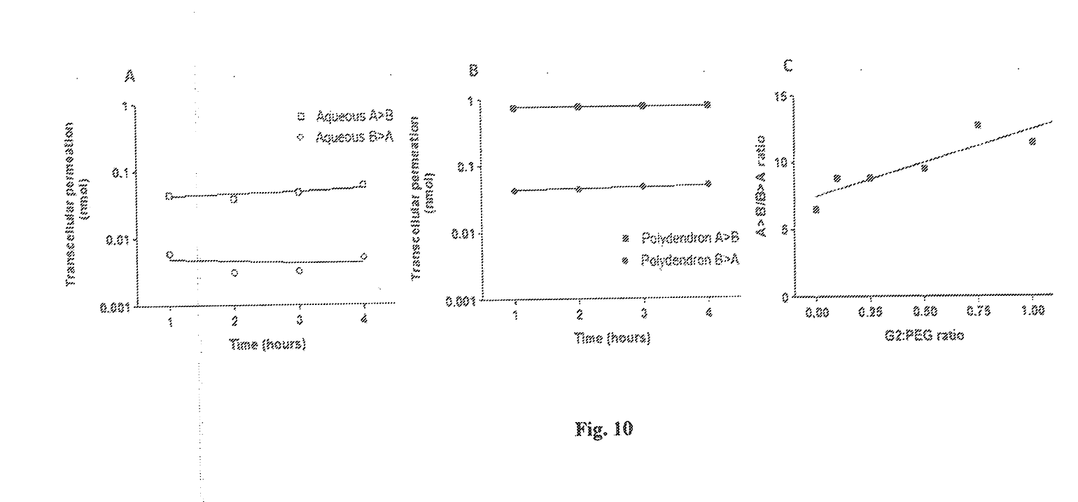

FIG. 10 shows results in relation to transcellular permeability of selected Nile Red polydendron materials across Caco-2 cell monolayers

FIG. 11 shows, schematically, how using different dendron:polyethylene glycol initiator ratios can result in a spectrum of hydrophobicity, amphiphilicity and hydrophilicity;

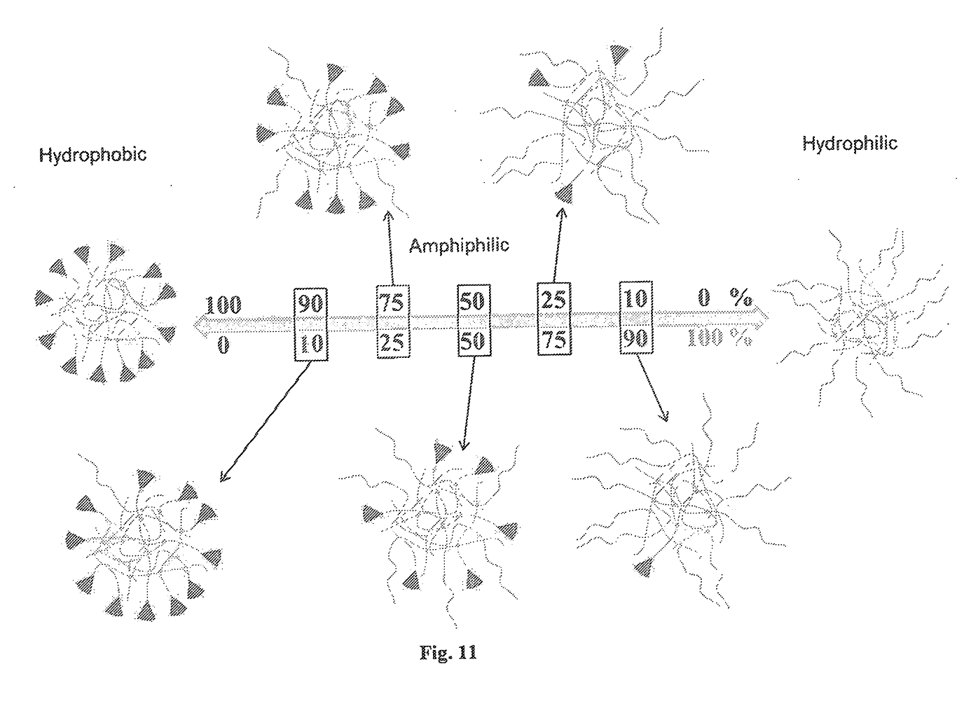

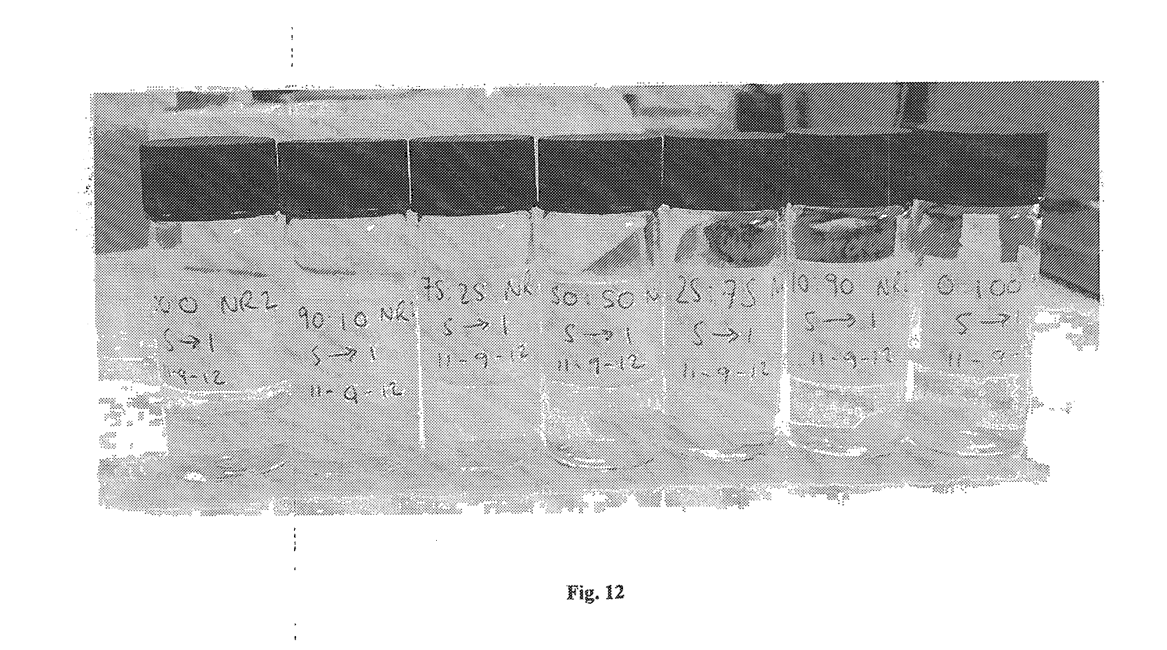

FIG. 12 is a photograph, corresponding to FIG. 11, and illustrates how using different dendron:polyethylene glycol initiator ratios can affect the response of encapsulated Nile Red;

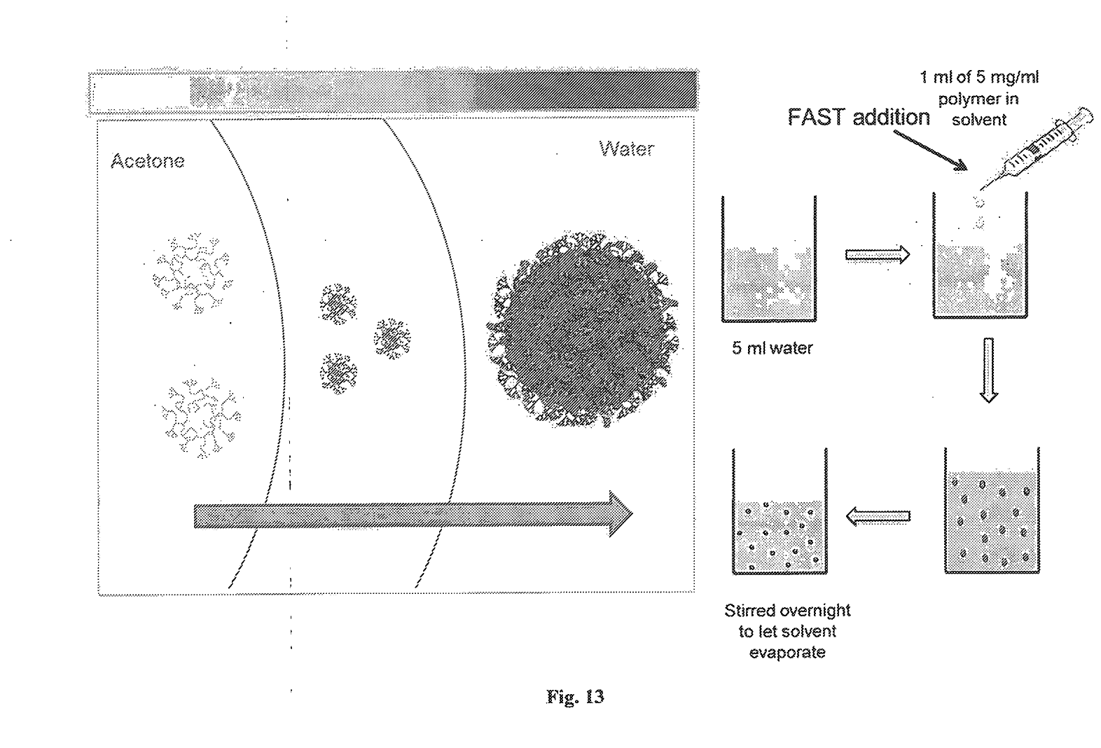

FIG. 13 shows, schematically, one method of nanoprecipitation of polydendrons;

FIGS. 14a and 14b are SEM images of polydendron nanoprecipitates;

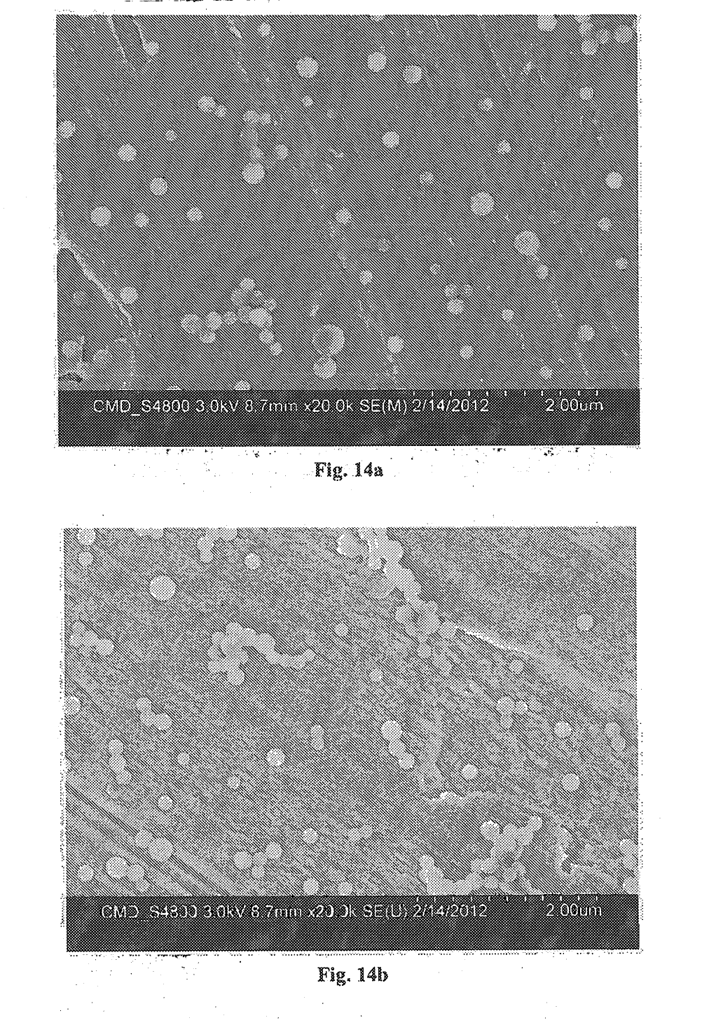

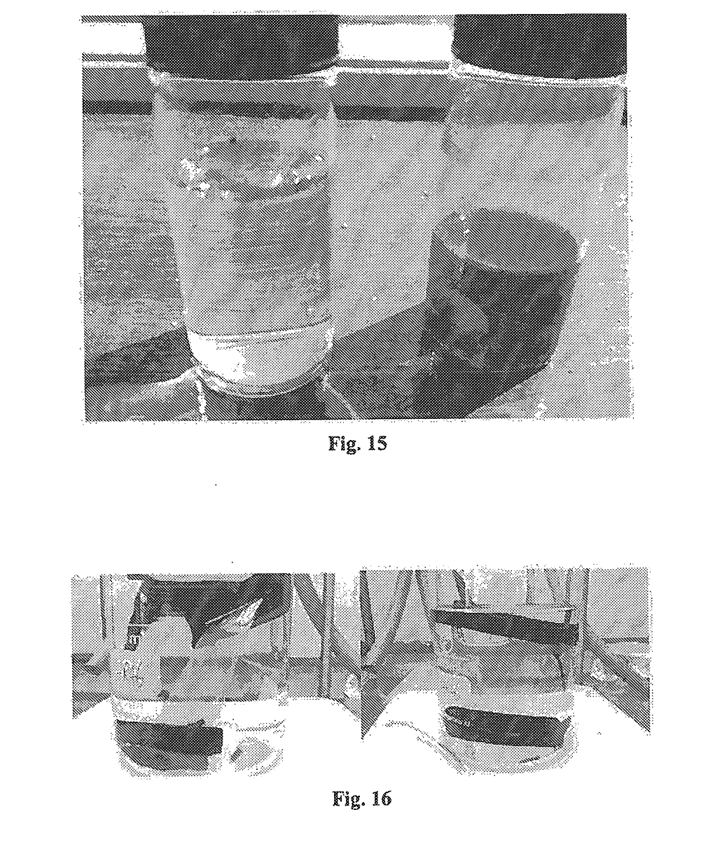

FIGS. 15 to 19 illustrate some effects of the polydendrons including pH responsive effects.

The experimental details below relate to: preparative procedures for various dendron and non-dendron initiators used in the present invention, including initiators containing polyethylene glycol (PEG) and sugar moieties; preparative procedures and properties of various polydendrons showing how hydrophilic or hydrophobic properties can be tailored and the effect of pH on these; nanoprecipitation methods and results; encapsulation experiments showing how molecules can be encapsulated and showing the effect of tailoring the encapsulation environment, as a model for drug encapsulation; cytotoxicity analysis using MTT and ATP assays in respect of Caco-2 cells; transcellular permeability of polydendrons carrying Nile Red (to model drug transfer across the intestinal epithelium); preparation of acid cleavable brancher; DEAEMA polydendron synthesis; hydrolysis of branched pDEAEMA; co-polydendron synthesis; nanoparticle formation, nile red encapsulation and fluoresceinamine encapsulation in respect of pH responsive polydendrons; encapsulation of inorganic material (e.g. magnetic particles); and illustrations of encapsulation, pH responsive effects, and behaviour in transport buffer.

The experimental details below disclose initiators and polymer backbones of various functionality including those having amine groups, the behaviour of which depends on pH.

It is clear that the methodology can be tailored to make polydendrons of each of the following four types:
Hydrophilic dendron/hydrophobic core
Hydrophobic dendron/hydrophilic core
Hydrophilic dendron/hydrophilic core
Hydrophobic dendron/hydrophobic core

Each of these four types have the capacity to be pH responsive.

Very positive results were obtained with regard to cytotoxicity and in the drug transport model. The experiments below show in particular that a material which would otherwise not pass effectively from gut to blood can be carried over by using polydendrons of the present invention.

Whereas a representation of an ideal dendrimer structure is shown in FIG. 5a, the present invention is concerned with polydendrons which have dendrons and a polymer core as represented in Figure Sc, constituent parts of which include dendrons attached to polymer chains as represented in FIG. 5b.

Polydendrons can be prepared by using mixed initiators, to end up with polydendron structures as represented for example in FIG. 11. At the far left of FIG. 11 is represented a hydrophilic polydendron made using 100% dendron initiator; at the far right of FIG. 11 is represented a hydrophobic material made using 100% PEG. The hydrophobicity/amphiphilicity/hydrophilicity can be tuned by varying the relative amounts of the different initiators.

FIG. 12 is a photograph of vials containing the seven different types of polydendron shown schematically in FIG. 11 (i.e. 100% dendron initiator with 0% PEG initiator on the left, through to 0% dendron initiator with 100% PEG initiator on the right) carrying Nile Red. In the original photograph, the darkest pink colour can be seen on the left, lighter pinks in the middle vials, and a very pale pink on the right, thereby showing that the hydrophobicity can be tuned in a discernible and controllable manner.

The present invention is focused on pH responsive polydendrons and pH responsive polydendron particles, aggregates and compositions, and methods of making them. Whilst some of the following examples disclose various components which are not in themselves pH responsive, nevertheless they may be used in combination with pH responsive components or features.

Novel products, components thereof intermediates, methods or method steps, disclosed herein, also fall within the scope of the present invention

### EXAMPLES

### i. Initiator Syntheses

1.1 Protected Sugar Initiator

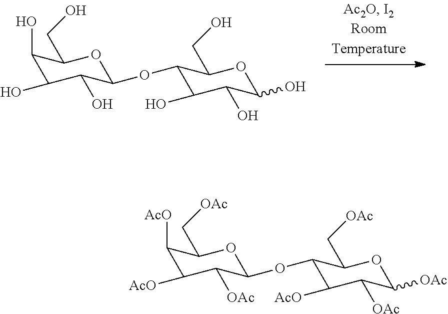 [SMILES: OCC1OC(OC2C(CO)OC(O)C(O)C2O)C(O)C(O)C1O ; CC(=O)OCC1OC(OC2C(COC(C)=O)OC(OC(C)=O)C(OC(C)=O)C2OC(C)=O)C(OC(C)=O)C(OC(C)=O)C1OC(C)=O]

Lactose (4 g, 11.7 mmol) was weighed into a 100 mL round bottom flask equipped with a magnetic stirrer and dry N₂ inlet. The flask was purged with nitrogen for 15 minutes. Acetic anhydride (30 mL) and Iodine (208 mg, 1.58 mmol) were added, instantly forming a brown coloured solution. Within 10 minutes the flask began to warm due to onset of acctylation. The solution was stirred overnight at room temperature under a positive flow of nitrogen. The solution was transferred to a 250 mL separating funnel containing dichloromethane (50 mL), sodium thiosulfate solution (30 mL) and crushed ice, and the product was extracted into the organic layer. The aqueous layer was further extracted with dichloromethane (2×50 mL). The organic phases were collected and washed with saturated sodium carbonate solution until neutral. The organic phase was collected, dried over anhydrous MgSO₄, and concentrated in vacuo to give a white solid.

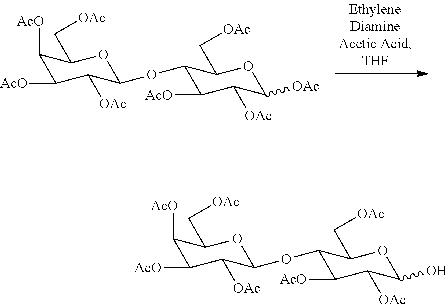 [SMILES: CC(=O)OCC1OC(OC2C(COC(C)=O)OC(OC(C)=O)C(OC(C)=O)C2OC(C)=O)C(OC(C)=O)C(OC(C)=O)C1OC(C)=O ; CC(=O)OCC1OC(O)C(OC(C)=O)C(OC(C)=O)C1OC1OC(COC(C)=O)C(OC(C)=O)C(OC(C)=O)C1OC(C)=O]

Lactose octa-acetate (5.1 g, 7.52 mmol) was weighed into a 250 mL round bottom flask equipped with a magnetic stirrer, and was dissolved in tetrahydrofuran (100 mL). Ethylene diamine (0.6 mL, 9.02 mmol) was added to the flask, followed by the slow addition of acetic acid (0.6 mL, 10.5 mmol), to give a white coloured turbid solution. A gas was evolved and the flask warmed slightly upon addition of the acid. The flask was lightly sealed with a rubber septum cap, and stirred overnight at room temperature, to give a cream coloured mixture. Distilled water (50 mL) was added to the flask, whereby the precipitate dissolved, leaving a slightly yellow coloured solution. The solution was transferred to a 500 mL separating funnel containing dichloromethane (100 mL), and the product was extracted into the organic solvent. A further extraction of the aqueous layer was performed with dichloromethane (50 mL). The organic layers were combined, washed with hydrochloric acid (80 mL, 2M), saturated sodium bicarbonate solution (80 mL) and distilled water (80 mL). The organic layer was dried over anhydrous MgSO₄, filtered and concentrated in vacuo. The crude product was purified by flash column chromatography (silica, eluent hexane/acetone, 60/40) to give a white solid.

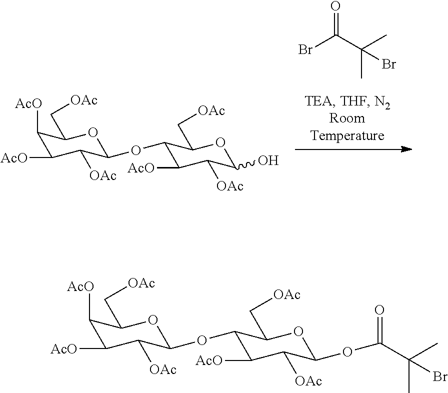

Lactose septa-acetate (3 g, 4.71 mmol) was added to a 50 mL round bottom flask equipped with a magnetic stirrer and dry N₂ inlet. The flask was then purged with nitrogen for 10 minutes. Anhydrous tetrahydrofuran (8 mL) was added to the flask, and N₂ was bubbled through the mixture for a further 10 minutes. Triethylamine (0.99 mL, 7.07 mmol) was added to a vial, diluted with tetrahydrofuran (2 mL), and then transferred to the reaction flask drop-wise. Following this, 2-bromoisobutyryl bromide (0.87 mL, 7.07 mmol) was added to a vial, diluted with tetrahydrofuran (2 mL) and transferred to the reaction flask drop-wise. Reaction mixture was left to stir overnight at room temperature under a positive flow of nitrogen. This gave a white coloured turbid mixture. The mixture was filtered by gravity filtration, the precipitate washed with tetrahydrofuran, and the solution concentrated in vacuo. The crude product was purified by flash column chromatography (silica, eluent hexane/ethyl acetate, 95/5) to give a white solid.

1.2 PEG Initiators

1.2.1 750-PEG Initiator

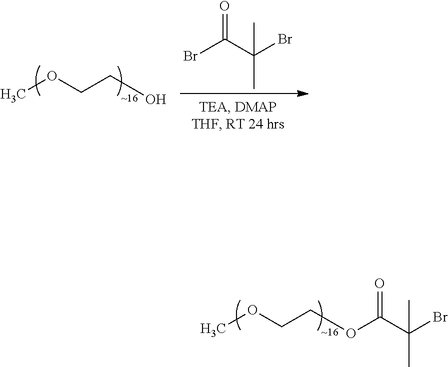

Monomethoxy poly(ethylene glycol) (Mw≈750 gmol⁻¹) (23.0 g, 30.7 mmol) was dissolved in warm THF (40° C.), and the reaction was degassed with dry N₂. DMAP (37.5 mg, 0.3 mmol) and TEA (7.48 ml, 53.7 mmol) were added and the reaction was cooled to 0° C. in an ice bath. α-brom isobutyryl bromide (5.69 ml, 46.0 mmol) was added dropwise over 30 minutes and a white precipitate appeared immediately; the Et₃NH⁺Br⁻ salt. After 24 hours the precipitate was filtered, THF removed in vacuo and the resulting crude product was precipitated from acetone into petroleum ether (30-40° C.) twice (72%). ¹H NMR (400 MHz, D₂O) δ ppm 4.31 (m, 2H), 3.77 (m, 2H), 3.70-3.59 (m, 60H), 3.55 (m, 2H), 3.31 (s, 3H) and 1.89 (s, 6H).

1.2.2 2K-PEG Initiator

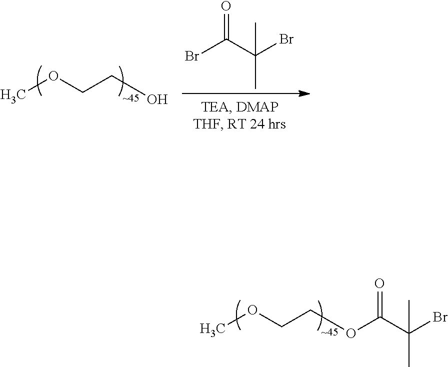

Monomethoxy poly(ethylene glycol) (Mw≈2000 gmol⁻¹) (20.5 g, 10.25 mmol) was dissolved in warm THF (40° C.), and the reaction was degassed with dry N₂. DMAP (12.5 mg, 0.1 mmol) and TEA (3.14 ml, 22.5 mmol) were added and the reaction was cooled to 0° C. in an ice bath. α-bromo isobutyryl bromide (2.53 ml, 20.5 mmol) was added dropwise over 20 minutes and a white precipitate appeared immediately; the Et₃NH⁺Br⁻ salt. After 24 hours the precipitate was filtered, THF removed in vacuo and the resulting crude product was precipitated from acetone into petroleum ether (30-40° C.) twice (89%). ¹H NMR (400 MHz, D₂O) δ ppm 4.34 (m, 2H), 3.80-3.59 (m, 186H), 3.35 (s, 3H) and 1.93 (s, 6H).

1.3 G0 (Non-Dendron) Initiators

1.3.1 G0 Tertiary Amine Functional Initiator

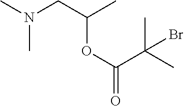

1-dimethylamino-2-propanol (1.1207 g, 10.86 mmol, 1 eq.), TEA (1.5390 g, 15.2 mmol, 1.4 eq.) and DMAP (132.7 mg, 1.086 mmol, 0.1 eq.) were added to a 250 mL 2 necked round-bottomed flask containing DCM (160 mL). The flask was deoxygenated under a positive N₂ purge for 10 minutes. α-bromoisobutyryl bromide (2.622 g, 1.4 mL, 11.4 mmol, 1.05 eq.) was added drop wise while the solution was stirring in an ice bath under a positive flow of N₂. The reaction mixture was allowed to warm to room temperature and left stirring overnight. The organic phase was washed with saturated sodium hydrogen carbonate (NaHCO₃) solution (3×30 mL). The solution was dried with anhydrous Na₂SO₄. ¹H NMR (400 MHz, CDCl₃) δ 1.27 (d, 3H), 1.89 (m, 6H), 2.17-2.55 (m, 8H), 5.07 (m, 1H). m/z (ES MS) 252 [M+H]⁺.

1.4 G1. G2 Dendron Initiators

1.4.1 G1-Aromatic Dendron Initiator (G1 DBOP Br)

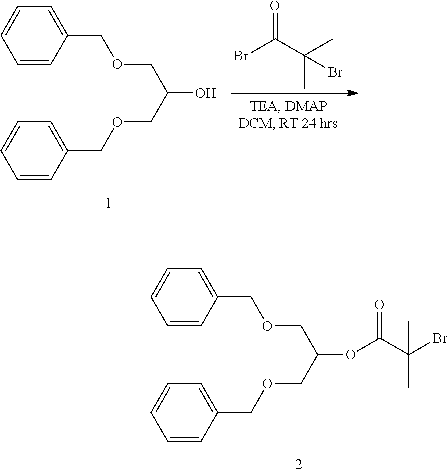

1,3-Dibenzyloxy-2-propanol, 1, (9.80 g, 36.0 mmol) was weighed into a 2-neck round bottom flask which was equipped with magnetic stirrer and dry N₂ inlet. Dichloromethane (DCM) (100 ml) was added followed by 4-(dimethylamino)pyridine (DMAP) (0.44 g, 3.6 mmol) and triethylamine (TEA) (7.53 ml, 54.0 mmol). The reaction was cooled to 0° C. in an ice-bath and α-bromoisobutyryl bromide (5.34 ml, 43.2 mmol) was added dropwise over 20 minutes. After complete addition the reaction was warmed to room temperature and left stirring overnight. Reaction could be observed by the formation of a white precipitate. After 24 hours the precipitate was removed by filtration, the resulting crude reaction medium was washed first with a saturated solution of NaHCO₃ (3×100 ml) followed by distilled water (3×100 ml). The organic layer was dried over Na₂SO₄ and concentrated in vacuo to give a pale yellow oil (81%). Found; C, 59.55; H, 6.02%. C₂₁H₂₅BrO₄ requires; C, 59.86; H, 5.98; Br, 18.96; O, 15.19%. ¹H NMR (400 MHz, CDCl₃) δ ppm 7.35-7.20 (m, 10H), 5.26 (m, 1H), 4.55 (m, 4H), 3.69 (d, 4H), 1.93 (s, 6H). ¹³C NMR (100 MHz, CDCl₃) δ ppm 171.2, 138.0, 128.4, 127.7, 127.6, 73.3, 68.5, 55.8, 30.7. m/z (ES MS) 443.1 [M+Na]⁺, 461.1 [M+K]⁺, m/z required 420.1 [M]⁺.

1.4.2 G2-Aromatic Dendron Initiator (G2 DBOP Br)

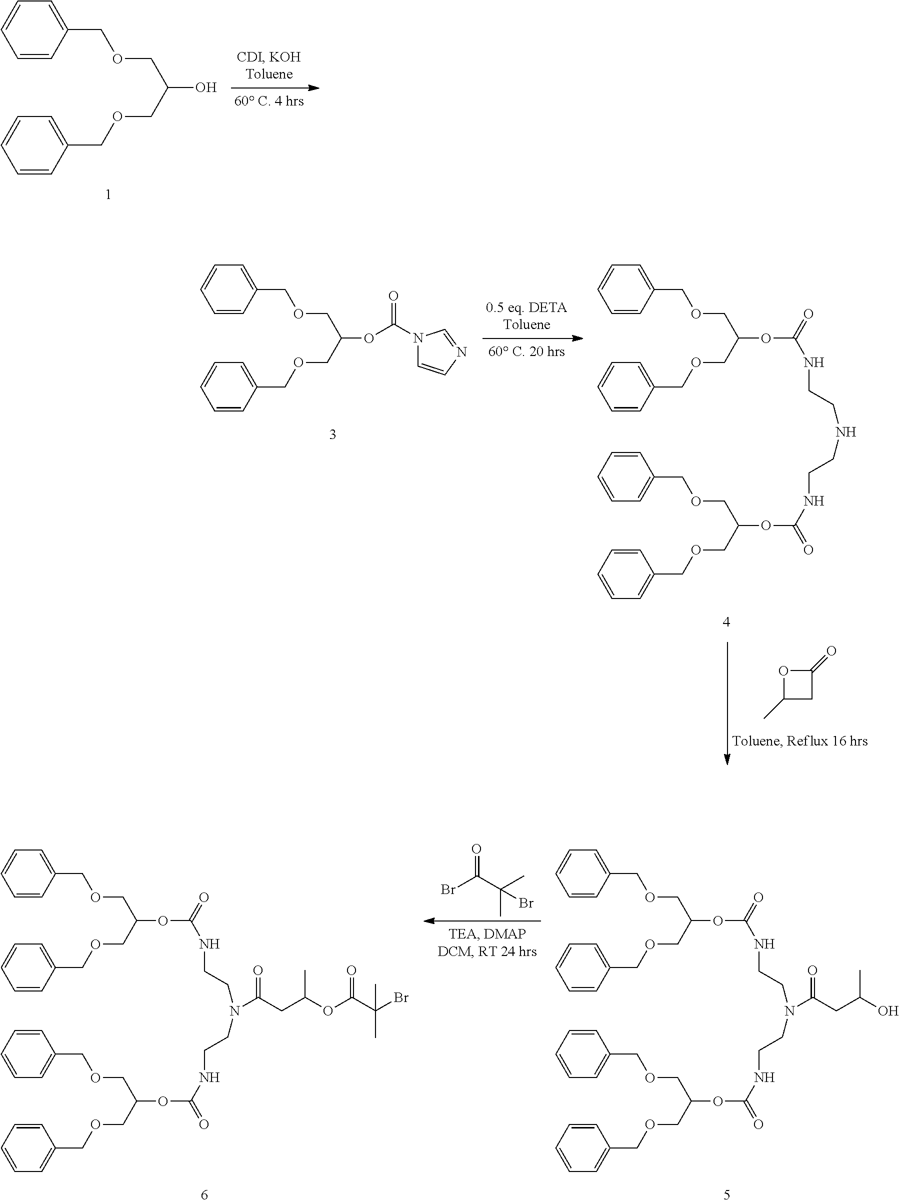

1,1′-Carbonyldiimidazole (CDI) (9.73 g, 60.0 mmol) was weighed into a 2-neck round bottom flask and equipped with magnetic stirring, condenser and dry N₂ inlet. Anhydrous toluene (100 ml) was added, followed by KOH (0.34 g, 6.0 mmol) and 1 (12.35 ml, 50.0 mmol). The reaction was heated to 60° C. for 6 hours. Toluene was removed in vacuo, the crude mixture was dissolved in DCM (50 ml) and washed with distilled water (3×50 ml). The organic layer was dried over Na₂SO₄ and concentrated in vacuo to give 3, a pale yellow oil (97%). Found; C, 68.64; H, 6.10; N, 7.85%. C₂₁H₂₂N₂O₄ requires C, 68.84; H, 6.05; N, 7.65; O, 17.47%. ¹H NMR (400 MHz, CDCl₃) δ ppm 8.11 (s, 1H), 7.41 (s, 1H), 7.33-7.23 (m, 10H), 7.06 (s, 1H), 5.36 (qn, 1H), 4.53 (m, 4H), 3.75 (m, 4H). ¹³C NMR (100 MHz, CDCl₃) δ ppm 148.3, 137.5, 137.2, 130.6, 128.4, 127.9, 127.6, 117.2, 76.1, 73.3, 68.1. m/z (ES MS) 367.2 [M+H]⁺, 389.2 [M+Na]⁺, 405.1 [M+K]⁺, m/z required 366.2 [M]⁺.

3 (16.84 g, 46.0 mmol) was weighed into a 2-neck round bottom flask which was equipped with magnetic stirring, condenser and dry N₂ inlet. Anhydrous toluene (120 ml) was added followed by diethylenetriamine (DETA) (2.48 ml, 23.0 mmol). The reaction was heated to 60° C. for 48 hours. Toluene was removed in vacuo, the resulting crude mixture was dissolved in DCM (100 ml) and washed with distilled water (3×100 ml). The organic layer was dried over Na₂SO₄ and concentrated in vacuo to give 4, a yellow oil (93%). Found; C, 68.50; H, 7.13; N, 6.00%. C₄₀H₄₉N₃O₈ requires; C, 68.65; H, 7.06; N, 6.00; O, 18.29%. ¹H NMR (400 MHz, CDCl₃) δ ppm 7.27-7.16 (m, 20H), 5.23 (s, br, NH), 5.03 (qn, 2H), 4.44 (m, 8H), 3.57 (d, 8H), 3.12 (m, 4H), 2.58 (m, 4H). ¹³C NMR (100 MHz, CDCl₃) δ ppm 156.6, 138.4, 128.8, 128.1, 73.7, 72.1, 69.4, 49.0, 41.2. m/z (ES MS) 700.4 [M+H]⁺, 722.3 [M+Na]⁺, 738.3 [M+K]⁺, m/z required 699.4 [M]⁺.

4 (15.01 g, 21.4 mmol) was weighed into a 2-neck round bottom flask, equipped with magnetic stirrer, condenser and dry N₂ inlet. Anhydrous toluene (90 ml) was added followed by dropwise addition of β-butyrolactone (2.62 ml, 32.2 mmol). The reaction was heated at reflux for 16 hours. Toluene was removed in vacuo, the resulting crude mixture was dissolved in DCM (50 ml) and washed with distilled water (3×50 mil). The organic layer was dried over Na₂SO₄ and concentrated in vacuo to give a yellow oil. The crude product was purified by silica gel column chromatography with a mobile phase gradient of DCM:MeOH (100:0-95:5-90:10) to give 5, a pale yellow oil (45%). Found; C, 65.35; H, 6.72; N, 5.10%. C₄₄H₅₅N₃O₁₀ requires; C, 67.24; H, 7.05; N, 5.35; O, 20.36%. ¹H NMR (400 MHz, CDCl₃) δ ppm 7.34-7.25 (m, 20H), 5.35 (br, NH), 5.31 (br, NH), 5.11 (m, 2H), 4.50 (m, 8H), 4.14 (s, 1H), 3.62 (m, 8H), 3.46-3.18 (m, br, 8H), 2.45-2.22 (m, 2H), 1.18-1.05 (m, 3H). ¹³C NMR (100 MHz, CDCl₃) δ ppm 174.4, 156.8, 156.6, 138.4, 138.3, 128.8, 128.1, 128.0, 73.7, 73.6, 72.6, 72.4, 69.5, 69.3, 65.1, 48.5, 46.5, 41.2, 40.3, 39.9, 22.9. m/z (ES MS) 808.4 [M+Na]⁺, m/z required 785.4 [M]⁺.

(9.31 g, 11.85 mmol) was dissolved in DCM (100 ml) and transferred to a round bottom flask which was equipped with magnetic stirring and a dry N₂ inlet. DMAP (0.14 g, 1.19 mmol), TEA (3.30 ml, 23.7 mmol) were added and the reaction mixture was cooled to 0° C. in an ice bath followed by dropwise addition of α-bromoisobutyryl bromide (2.19 ml, 17.78 mmol). The reaction was warmed to room temperature for 24 hours. A colour change from pale orange to a dark orange/brown colour was observed over time. No precipitate was observed, the crude reaction mixture was washed with a saturated NaHCO₃ solution (3×100 mil) and distilled water (3×100 ml). The organic layer was dried over Na₂SO₄ and concentrated in vacuo to give 6, an orange oil (81%). Found; C, 59.50; H, 6.31; N, 4.39%. C₄₈H₆₀BrN₃O₁₁ requires; C, 61.67; H, 6.47; Br, 8.55; N, 4.49; O, 18.82%. ¹H NMR (400 MHz, CDCl₃) δ ppm 7.35-7.23 (m, 20H), 5.33 (s, br, NH), 5.10 (m, 2H), 4.52 (m, 8H), 3.71-3.53 (s, 8H), 3.52-3.12 (m, br, 8H), 2.76 (d of d, 1H), 2.47 (d of d, 1H), 1.87 (s, 6H), 1.29 (d, 3H). ¹³C NMR (100 MHz, CDCl₃) δ ppm 192.5, 170.8, 156.3, 156.1, 137.9, 134.5, 128.4, 127.7, 127.6, 73.2, 73.1, 72.2, 71.8, 70.2, 69.1, 69.0, 68.8, 56.1, 48.3, 46.3, 39.6, 39.4, 38.9, 30.8, 30.7, 30.6, 19.7. m/z (ES MS) 958.3 [M+Na]⁺, 974.3 [M+K]⁺, m/z required 933.3 [M]⁺.

1.4.3 Alternative G2 DBOP Br Synthesis

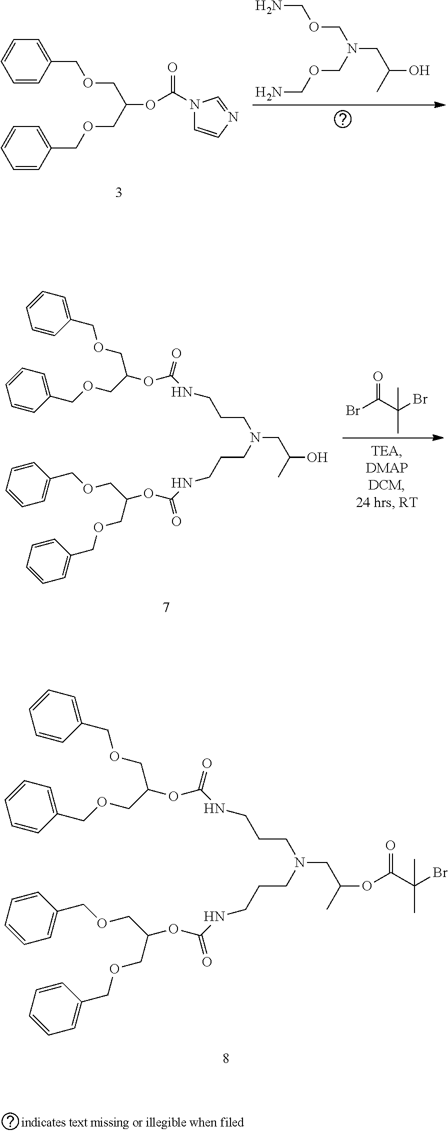

3 (14.03 g, 38.3 mmol) was added to a 2-neck round bottom flask, which was equipped with magnetic stirring, condenser and a N₂ inlet. Anhydrous toluene (100 ml) was added and the reaction was heated to 60° C. The AB₂ brancher (3.627 g, 19.2 mmol) was dissolved in anhydrous toluene (5 ml) was added dropwise. After 18 hours the reaction was stopped, the toluene removed in vacuo, the crude mixture was dissolved in dichloromethane (100 ml) and washed with water (3×100 ml). The organic phase was dried over Na₂SO₄ the solvent removed in vacuo and the resulting yellow oil was dried further under high vacuum to give 7, as a pale yellow oil, (94%). ¹H NMR (400 MHz, CDCl₃) δ ppm 7.33-7.23 (m, 20H), 5.30 (s, br, NH), 5.09 (m, 2H), 4.51 (m, 8H), 3.73 (m, 1H), 3.64 (d, 8H), 3.16 (m, 4H), 2.53 (m, 2H), 2.32 (m, 2H), 2.24 (m, 2H), 1.59 (m, 4H), 1.06 (d, 3H). m/z (ES MS) 786.4 [M+H]⁺, 808.4 [M+Na]⁺, m/z required 785.43 [M]⁺.

7, (13.381 g, 17.0 mmol) was dissolved in DCM (100 ml) and bubbled with N₂ for 20 minutes. 4-(Dimethylamino)pyridine (DMAP) (21 mg, 0.17 mmol) and triethylamine (TEA) (3.56 ml, 26.0 mmol) were added and the reaction vessel was cooled to 0° C. α-Bromoisobutyryl bromide (2.53 ml, 20.0 mmol) was added dropwise, then the reaction was warmed to room temperature for 24 hours. The organic phase was washed with a saturated solution of NaHCO₃ (3×150 ml) and distilled water (3×150 ml), dried over Na₂SO₄ and the solvent removed in vacuo to give an orange oil as the crude product. This was purified by column chromatography with a silica stationary phase and mobile phase of ethyl acetate:hexane (4:1), to give 8 a yellow oil, (73%). Found; C, 63.24; H, 6.88; N, 4.44%. C₄₉H₆₄BrN₃O₁₀ requires; C, 62.95; H, 6.90; N, 4.49%. ¹H NMR (400 MHz, CDCl₃) δ ppm 7.33-7.24 (m, 20H), 5.36 (s, br, NH), 5.09 (m, 2H), 5.03 (m, 1H), 4.51 (m, 8H), 3.64 (d, 8H), 3.16 (m, 4H), 2.64-2.35 (m, 6H), 1.89 (s, 6H), 1.60 (m, 4H), 1.22 (d, 3H). ¹³C NMR (100 MHz, CDCl₃) δ ppm 171.2, 156.0, 138.1, 128.3, 127.60, 127.62, 73.2, 71.6, 70.4, 68.9, 59.1, 56.1, 52.2, 39.4, 30.6, 30.7, 27.2, 18.0. m/z (ES MS) 936.4 [M+H]⁺, 959.4 [M+Na]⁺, m/z required 935.4 [M]⁺.

1.4.4 G1, G2 tBOC Dendron Initiator Synthesis (Inc. AB₂ Synthesis)

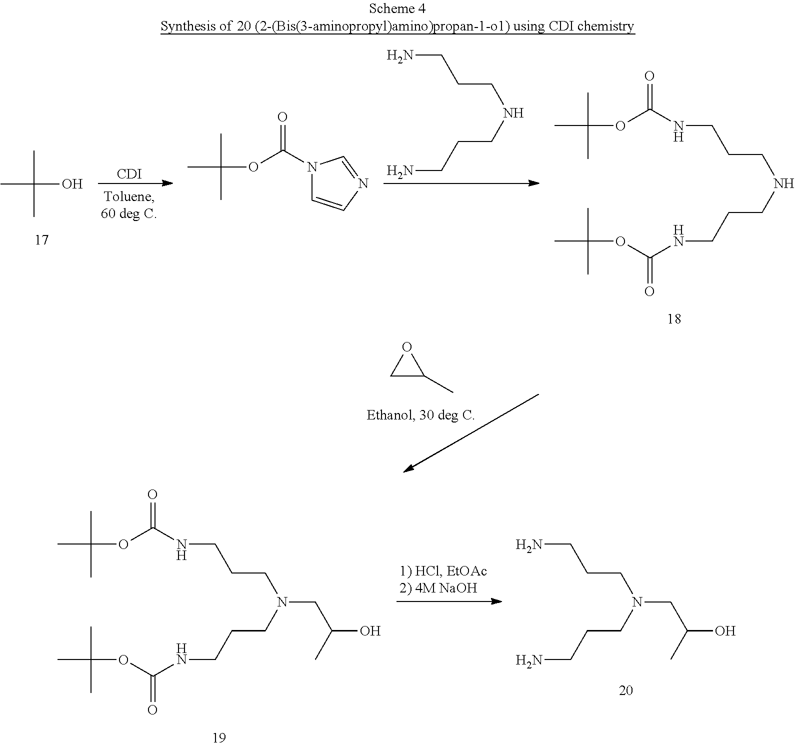

Synthesis of 18—CDI (39.137 g, 0.241 mol) was added to an oven-dried 500 mL 2-neck RBF fitted with a reflux condenser, magnetic stirrer and a dry N₂ inlet. Dry toluene (350 mL) was added and the flask was purged with N₂ for 10 minutes. The solution was stirred at 60° C. and 17 (t-Butanol) (35.7 g, 46 mL, 0.483 mol) was added via a warm syringe. The mixture was left stirring at 60° C. for 6 hours under a positive flow of nitrogen. Following this, BAPA (16.077 g, 17.14 mL, 0.121 mol) was added dropwise. The reaction was left stirring for a further 18 hours at 60° C. under a positive flow of nitrogen, and then allowed to cool to room temperature. The pale yellow solution was filtered to remove any solid imidazole, and concentrated In vacuo. The remaining oil was dissolved in dichloromethane (250 mL) washed with distilled water (3×250 mL) and finally a saturated brine solution (150 mL). The organic layer was dried with anhydrous Na₂SO₄, filtered and concentrated in vacuo to give 18 as a white solid powder. 38 g, (95%). Found; C, 57.84; H, 10.45; N, 12.91%. Cl₆H₃₃N₃O₄ requires; C, 57.98; H, 10.04; N, 12.68%. ¹H NMR (400 MHz, CDCl₃) 5.19 (s, br, NH—disappears on addition of D₂O), 3.21 (t, 4H), 2.65 (t, 4H), 1.65 (q, 4H), 1.44 (s, 18H) ¹³C NMR (100 MHz, CDCl₃) 156.48, 79.34, 47.77, 39.29, 30.11, 28.79. m/z (ES MS) 332.3 [M+H]⁺

Synthesis of 19-18 (20 g, 0.06 mol) was added to a 500 mL 2-necked RBF fitted with a reflux condenser, magnetic stirrer and a dry N₂ inlet. The flask was degassed with dry nitrogen for 10 minutes, and dissolved in dry ethanol (200 mL), Whilst stirring, and maintaining the temperature at 30° C., propylene oxide (10.51 g, 11.21 mL, 0.181 mol) was added dropwise over a period of 10 minutes. Under a positive flow of dry N₂, the reaction was left stirring at 30° C. for 18 hours. After this time, the solvent and excess propylene oxide were removed in vacuo. The crude product was purified by liquid chromatography on silica gel, eluting with EtOAc:MeOH, 4:1, the solvent removed in vacuo to give 19 as a pale yellow viscous oil 19.90 g, (85%). Found; C, 58.50; H, 10.23; N, 10.82%. C₁₉H₃₉N₃O₅ requires; C, 58.58; H, 10.09; N, 10.79%. ¹H NMR (400 MHz, CDCl₃) 4.93 (s, br, NH), 3.76 (m, 1H), 3.15 (m, 4H), 2.61-2.88 (m, 6H), 1.62 (m, 4H), 1.44 (s, 18H), 1.11 (d, 3H). ¹³C NMR (100 MHz, CDCl₃) 156.08, 79.18, 63.45, 62.55, 51.77, 38.75, 27.48, 20.14. m/z (ES MS) 390.3 [M+H]⁺

Synthesis of 20 (Part 1)—In a IL RBF, G1-OH (33.70 g) was dissolved in ethyl acetate (330 mL) and concentrated HCl (35.03 g, 30 mL, d=1.18 36% active) was added very slowly. CO₂ began to evolve. The reaction vessel was left open and stirring for 6 hours. ¹H NMR (D₂O) confirmed complete decarboxylation. Synthesis of 20 (Part 2)—After removal of ethyl acetate, the crude oil was dissolved in 4M NaOH (300 mL), and then reduced down by half (approx.) on the rotary evaporator (60° C.). Following this, the oily mixture was extracted twice with CHCl₃ (300 mL). The organic layers were then combined, dried with anhydrous Na₂SO₄, filtered and concentrated in vacuo to give the product as a pale yellow oil (15.27 g, 94% yield) NMR (400 MHz, CDCl₃) 3.79 (m, 1H), 2.68-2.40 (ddd, 2H), 2.31 (m, 4H), 1.89 (s, br, OH), 1.60 (m, 4H), 1.11 (d, 3H). ¹³C NMR (100 MHz, CDCl₃) 63.95, 62.56, 52.10, 40.31, 30.80, 20.03

Preparation of t-BOC G2 Dendron, 21

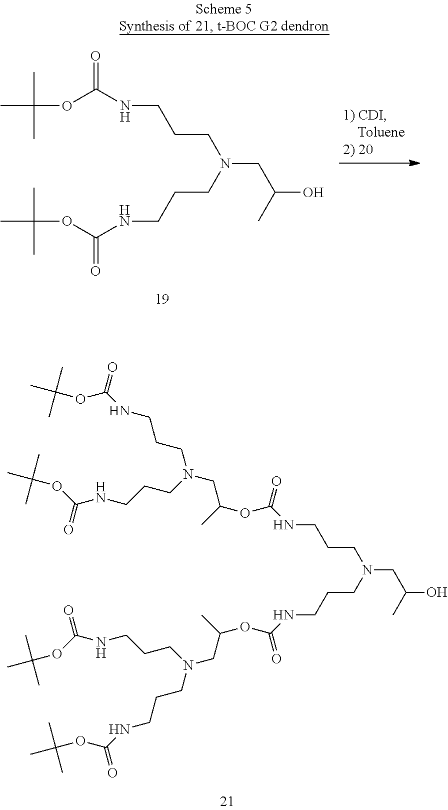 [SMILES: CC(O)CN(CCCNC(=O)OC(C)(C)C)CCCNC(=O)OC(C)(C)C ; CC(O)CN(CCCNC(=O)OC(C)CN(CCCNC(=O)OC(C)(C)C)CCCNC(=O)OC(C)(C)C)CCCNC(=O)OC(C)CN(CCCNC(=O)OC(C)(C)C)CCCNC(=O)OC(C)(C)C]

Synthesis of 21-19 (5 mL, 12.8 mmol) was added to a 250 mL 3 necked round bottom flask containing dry toluene (60 mL), which was fitted with a reflux condenser, magnetic stirrer and a dry N₂ inlet. The flask was purged with N₂ for 10 minutes. The solution was stirred at room temperature and CDI (2.29 g, 14.1 mmole) was added via a powder addition funnel. The mixture was heated to 60° C. with stirring for 6 hours. 20 (0.91 mL, 6.4 mmole) was added dropwise whilst the solution was stirring and the temperature was maintained at 60° C. The reaction was left overnight stirring for a further 12 hours at 60° C., and then allowed to cool to room temperature. The clear solution was filtered to remove any solid imidazole, and concentrated in vacuo. The crude product was purified by liquid chromatography, silica gel, eluting with EtOAc:MeOH, 5:1, the solvent removed in vacuo to give 21 as a pale yellow viscous oil (60%). Found; C, 57.46; H, 9.83; N, 12.17%. C₁₉H₃₉N₃O₅ requires; C, 57.68; H, 9.58; N, 12.35%. ¹H NMR (400 MHz, CDCl₃) 4.92 (m, br, 2H), 3.74 (m, 1H), 3.35-2.93 (m, 12H, 2.73-2.14 (m, 18H), 1.62 (m, 12H), 1.44 (s, 36H), 1.20 (m, 6H), 1.10 (d, 3H) ¹³C NMR (100 MHz, CDCl₃) 156.76, 156.15, 78.91, 67.58, 63.51, 62.46, 59.36, 52.33, 51.75, 38.94, 28.50, 27.37, 20.13, 18.82, 14.20. (ES MS) 1020.7 [M+H]⁺, 1042.7 [M+Na].

Synthesis of t-BOC Initiators 22 and 23

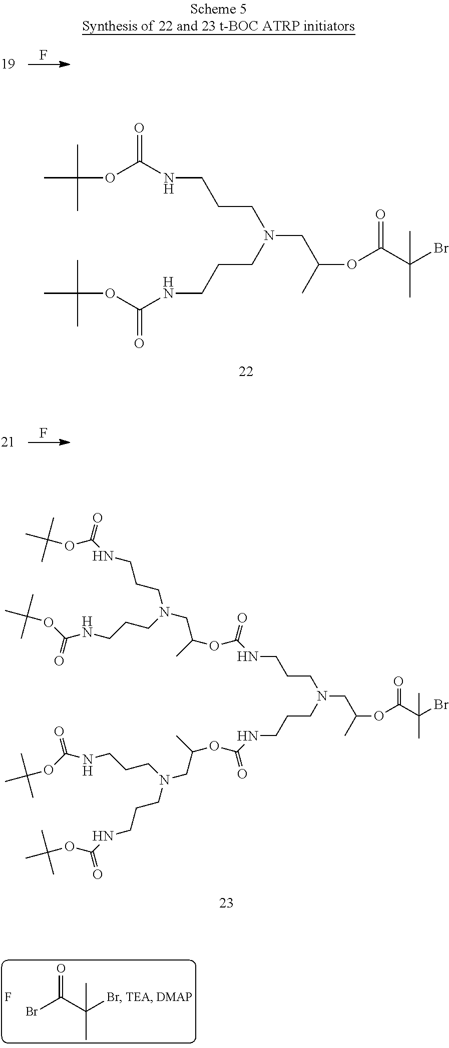

General Procedure for Focal Point Modification to ATRP Initiator by Acid Bromide—

19 or 20 was added to a 50 mL round bottom flask, which was equipped with a magnetic stirrer and purged with dry N₂ for 10 minutes. Following this, dichloromethane (40 mL), DMAP (0.2 eqv.) and TEA (2 eqv.) were also added. The round bottom flask was then purged again with dry N₂, and placed into an ice bath. Dropwise, over a period of 10 minutes 2-Bromoisobutyryl bromide (1.1 eqv.) was added. The reaction was removed from the ice bath after 30 minutes and left for 24 hours at room temperature. A colour change from clear to yellow/orange was noted for all reactions. After this time, the solution was filtered, washed with distilled water (3×40 mL), washed with a saturated brine solution (40 mL) and the organic layer dried using anhydrous Na₂SO₄. The solvent was removed in vacuo, and the crude product purified by column chromatography

Synthesis of 22-19, Bromoisobutyryl bromide (1.1 eqv.), DMAP (0.2 eqv) and TEA (2 eqv) were allowed to react according to the general esterification procedure above in 100 mL of dry CH₂C₂ for 24 h. The crude product was purified by liquid chromatography on silica gel, eluting with 95/5 DCM/MeOH increasing to 90/10 DCM/MeOH to give 22 as a light yellow/brown viscous oil. (77%) ¹H NMR (400 MHz, CDCl₃) 5.06 (s, br, NH), 3.15 (m, 4H), 2.68-2.35 (m, 6H), 1.93 (s, 6H), 1.61 (q, 4H), 1.43 (s, 18H), 1.25 (d, 3H) ¹³C NMR (100 MHz, CDCl₃) 171.81, 156.05, 79.57, 70.78, 59.62, 56.36, 38.65, 31.14, 30.17, 27.36, 18.26. m/z (ES MS) 510.2 [M+H]⁺, 534.2 [M+Na]⁺, 550.2 [M+K]⁺

Synthesis of 23-20, Bromoisobutyryl bromide (1.1 eqv.), DMAP (0.2 eqv) and TEA (2 eqv) were allowed to react according to the general esterification procedure above in 100 mL of dry CH₂Cl₂ for 24 h. The crude product was purified by liquid chromatography on silica gel, eluting with 85:15 CCl₃/MeOH to give 23 as a brown viscous oil. (54%) ¹H NMR (400 MHz, CDCl₃) 4.92 (m, br, 2H), 3.63 (m, 1H), 3.37-2.94 (m, 12H), 2.77-2.12 (m, 18H), 1.91 (s, 6H), 1.62 (m, 12H), 1.44 (s, 36H), 1.20 (m, 9H) m/z (ES MS) 1168.7 [M+H]⁺, 1192.7 [M+Na], 1208.7 [M+K]⁺

1.4.5 G1 Xanthate Dendron Initiator Synthesis (Xant-G1)

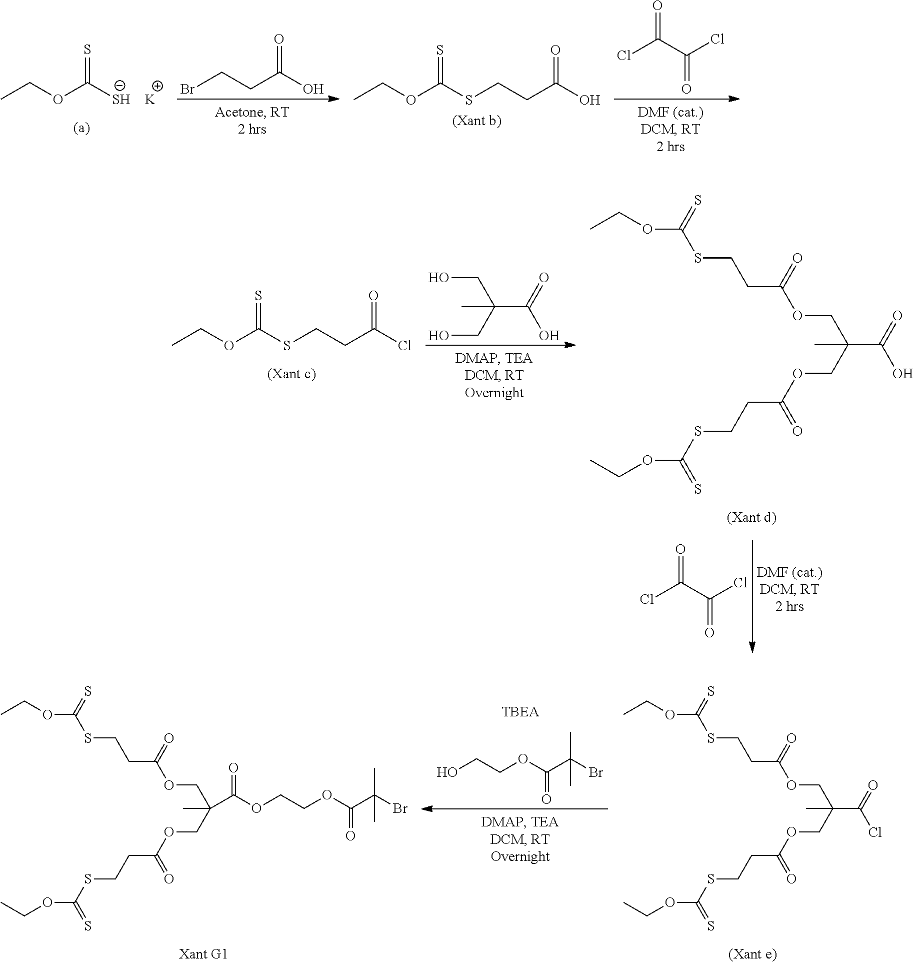

Synthesis of Xant b (Scheme 5)—

Potassium ethyl xanthogenate (40.1 g, 250.2 mmol) was transferred to a 500 mL two-necked round-bottomed flask, equipped with a magnetic stirrer bar, dropping funnel and septa cap with outlet. Acetone (150 mL) was added to the flask. 3-Bromopropionic acid (32.4 g, 211.8 mmol) was dissolved in acetone (80 mL) and transferred to dropping funnel. The acid was added to the flask dropwise with stirring. Once added, the reaction was left stirring at room temperature overnight. The initially yellow solid turns white as the reaction proceeds. The white solid is then filtered off and the solvent removed on the rotary evaporator. The resulting solid was dissolved in DCM (300 mL) and washed (1×200 mL distilled water and 2×200 mL brine). The organic layer was dried over MgSO₄, and the solid filtered off. The solvent was removed and placed in a vacuum oven to remove any residual solvent. Yield 59%. ¹H NMR (400 MHz, CDCl₃) δ: 1.42 (t, 3H), 2.85 (t, 2H), 3.38 (t, 2H), 4.63 (q, 2H)

Synthesis of Xant c (Scheme 5)—

Xanthate carboxylic acid, Xant b in (scheme 5) (15.0 g, 77.2 mmol) was transferred to a 250 mL round-bottomed flask, equipped with a magnetic stirrer bar and septa cap containing outlet. DCM (100 mL) was added. 5 drops of DMF was added. Oxalyl chloride (19.6 g, 154.4 mmol) was added dropwise via syringe with stirring. The reaction was left stirring for 2 hours. The reaction mixture changes from clear to a transparent orange as the reaction proceeds. The solvent was removed and washed twice with chloroform to remove any residual oxalyl chloride. Resulting viscous orange oil used as obtained. Yield quantitative. ¹H NMR (400 MHz, CDCl₃) δ: 1.42 (t, 3H), 3.38 (m, 4H), 4.63 (q, 2H).

Synthesis of Xant d (Scheme 5)—

Bis-MPA (4.1 g, 30.9 mmol), TEA (12.9 mL, 101.2 mmol) and DMAP (188.6 mg, 1.6 mmol) were transferred to a 250 mL two-necked) round-bottomed flask equipped with a magnetic stirrer bar, dropping funnel and septa cap containing outlet. The flask was then deoxygenated using nitrogen. Dry DCM (60 mL) was added via syringe under nitrogen. Xanthate acid chloride, Xant c in (scheme 5) . . . (16.4 g, 77.2 mmol) was degassed with nitrogen inside the sealed dropping funnel. Dry DCM (10 mL) was added to dissolve the acid chloride. The xanthate acid chloride was added dropwise and the reaction was left stirring under nitrogen overnight. The resulting solution was washed (1×200 mL distilled water and 2×200 mL brine). The organic layer was dried over MgSO₄, and the solid filtered off. The solvent was reduced and the product was run through an automated flash column with a starting eluent of 95:5 hexane: ethyl acetate increasing to 20:80.

Product fractions collected and solvent removed. The product was further washed with chloroform to remove residual ethyl acetate, and solvent removed again. Resulting oily product was placed in vacuum oven to remove any residual solvent.

Yield (35%). ¹H NMR (400 MHz, CDCl₃) δ: 1.30 (s, 3H), 1.42 (t, 6H), 2.80 (t, 4H), 3.37 (t, 4H), 4.30 (m, 4H), 4.65 (q, 4H).

Synthesis of Xant e (Scheme 5)—

Xant d (scheme 5) (4.8 g, 9.9 mmol) was transferred to a 100 mL round-bottomed flask equipped with a magnetic stirrer bar and septa cap containing outlet. DCM (30 mL) was added. 5 drops of DMF were added. Oxalyl chloride (2.5 g, 19.8 mmol) was added dropwise via syringe. The reaction was left stirring for 3 hours. The solution changed from pale yellow to dark orange as the reaction proceeds. The solvent was removed and the resulting oil was washed twice with chloroform to remove any residual oxalyl chloride. The product was in the form of viscous brown oil. Yield quantitative. ¹H NMR (400 MHz, CDCl₃) δ: 1.42 (m, 9H), 2.80 (t, 4H), 3.38 (t, 4H), 4.35 (m, 4H), 4.65 (q, 4H).

Synthesis of Xant-G1 (Scheme 5)—

Tertiary-bromoester alcohol (TBEA in scheme 5)(1.8 g, 8.6 mmol), TEA (1.8 mL, 12.9 mmol) and DMAP (52.6 mg, 0.4 mmol) were transferred to a 100 mL two-necked round-bottomed flask, equipped with a magnetic stirrer bar, dropping funnel and septa cap containing outlet. The flask was then deoxygenated using nitrogen. Dry DCM (30 mL) was added via syringe under nitrogen. Xant e (5.0 g, 9.9 mmol) was deoxygenated using nitrogen inside the sealed dropping funnel. Dry DCM (10 mL) was added via syringe. Xant e was added dropwise. The flask was cooled in an ice bath during this addition. The reaction was left stirring overnight. The resulting brown solution was washed (1×80 mL distilled water and 2×80 mL brine). The organic layer was dried over MgSO₄, and the solid filtered off. The solvent was reduced and the product was run through an automated flash column with a starting eluent of 100:0 hexane: ethyl acetate increasing to 20:80. Product fractions collected and solvent removed. The product was further washed with DCM to remove residual ethyl acetate, and solvent removed again. The resulting yellow/brown oil was left in a high vacuum vessel overnight to remove any residual solvent. Yield (40%). ¹H NMR (400 MHz, CDCl₃) δ: 1.28 (s, 3H), 1.43 (t, 6H), 1.95 (s, 6H), 2.78 (t, 4H), 3.37 (t, 4H), 4.25 (m, 4H), 4.42 (m, 4H), 4.65 (q, 4H).

Mass spec: m/z=703.0 [M+Na]⁺.

1.4.6 G1, G2, G3 Xanthate Dendron Synthesis Using bisMPA Backbone

For key references relating to the synthesis of bis-MPA dendrimers, refer to the following:
Macromolecules 2002, 35, 8307-8314
J Am. Chem. Soc., 2001, 123, 5908-5917
J. Am. Chem. Soc., 2009, 131, 2906-2916

For preparation of benzylidene protected bis-MPA anhydride follow:
J. Am. Chem. Soc., 2001, 123, 5908-5917

For preparation of DPTS 4-(Dimethylamino)pyridinium 4-toluenesulfonate follow:
J. S. Moore, S. I. Stupp, Macromolecules, 1990, 23, 65

For preparation of 2-hydroxyethyl 2-bromo-2-methylpropanoate follow:
J. Mater. Chem., 2011, 21, 18623-18629

Preparation of Xanthate Based Carboxylic Acid Building Block

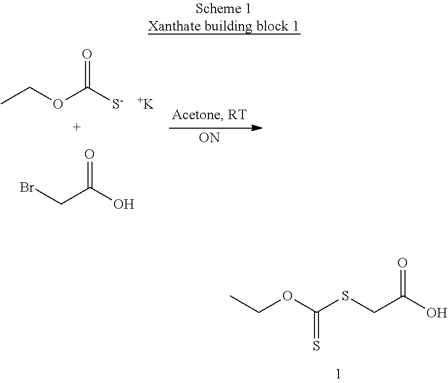

Synthesis of 2-((Ethoxycarbonothioyl)thio)acetic acid 1—A 500 mL round-bottomed flask equipped with a dropping funnel was charged with a magnetic stirrer bar, potassium ethyl xanthogenate (26.77 g, 167 mmol), and acetone (75 mL). A solution of 2-bromoacetic acid (19.31 g, 103 mmol) in acetone (40 mL) was added dropwise at room temperature over a period of 60 min. Stirring was continued overnight at room temperature. Solids were removed by filtration to afford a clear pale yellow solution. The solids on the funnel were washed with acetone (total of 50 mL). The combined washing and filtrate solutions were concentrated under vacuum to furnish a yellow viscous liquid that was dissolved in dichloromethane (150 mL). This solution was washed twice with brine (100 mL), and the organic phase was dried over MgSO4 and evaporated to dryness to afford 18.75 g (75%) of a white solid. ¹H NMR (400 MHz, CDCl₃): δ=1.43 (t, J=7.32 Hz, 3H), 3.98 (s, 2H) 4.67 (q, J=7.25 Hz, 2H), 4.53 ¹³C NMR (100 MHz, CDCl₃): δ=13.68, 37.60, 70.93, 174.30, 212.01

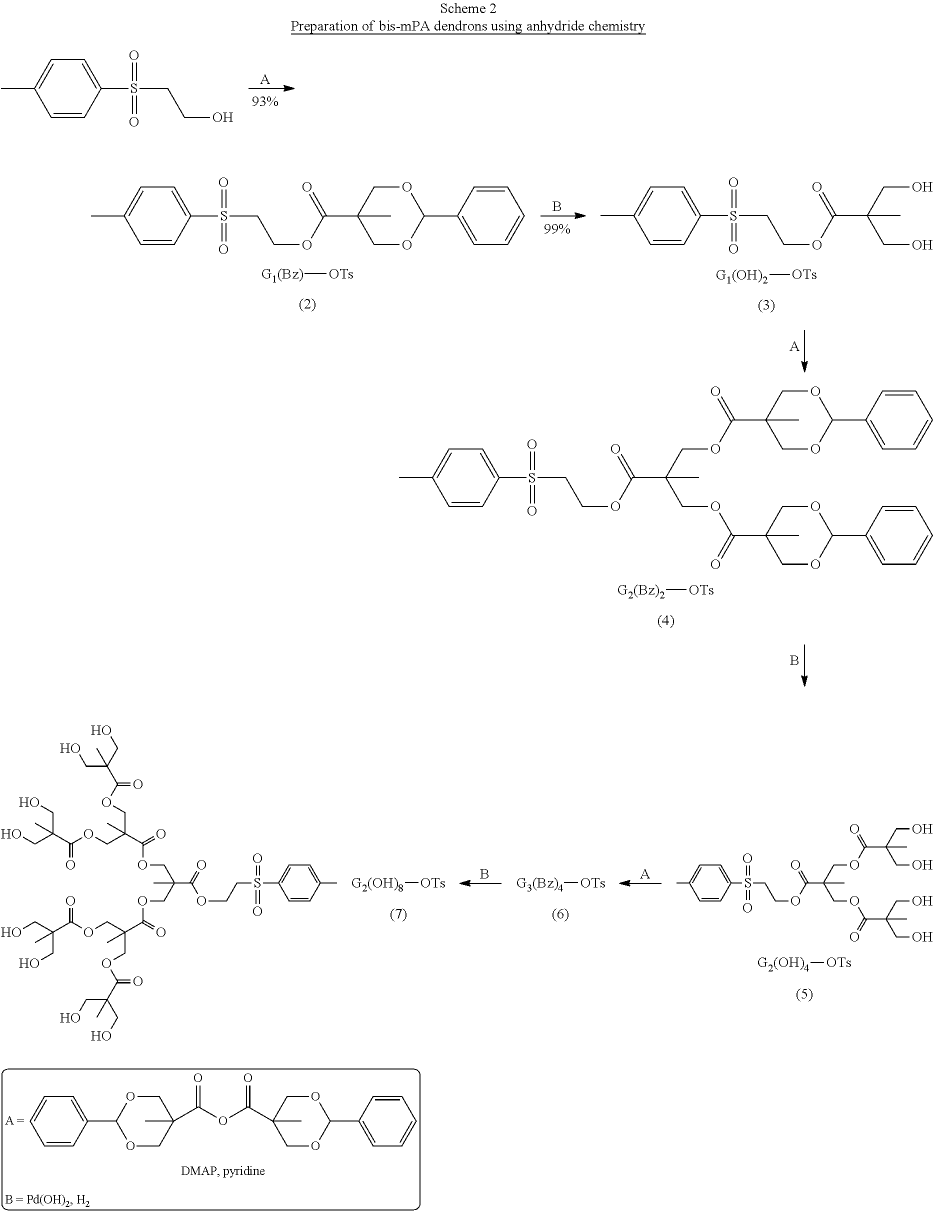

General Procedure for Dendon Growth (2, 4 and 6)—

To a 500 mL oven-dried round-bottom flask equipped with a magnetic stirrer (under nitrogen atmosphere), the benzylidene protected anhydride, the hydroxyl-terminated dendron (generation 0 through to 3), and 4-dimethylaminopyridine (DMAP) were all dissolved in a 1:1 ratio of CH₂Cl₂: pyridine (v/v). After stirring at room temperature for over 12 h, approximately 2 mL of water was added and the reaction was stirred for an additional 18 h in order to quench the excess anhydride. The product was isolated by diluting the mixture with CH₂C₂ (150 mL) and washing with 1 M NaHSO4 (3×150 mL), saturated aqueous NaHCO₃ (2×150 mL), and brine (150 mL). The organic layer was dried over MgSO4 and evaporated to dryness. Any residual solvent was removed under high vacuum overnight to yield a white foam with a typical yield greater than 95%.

General Procedure for Deprotection of Benzylidene by Hydrogenation (3, 5 and 7)—

To a reactor suitable for medium pressure hydrogenation fitted with a magnetic stirrer, the benzylidene protected dendrimer was dissolved in a 1:1 mixture of CH₂Cl₂: MeOH (v/v). Pd(OH)₂ on carbon (20%) was added and the reactor was evacuated and back-filled with hydrogen three times (H₂ pressure:10 bar). After vigorous stirring for 16 h, the reaction mixture was filtered through celite using a Buchner funnel and the filtrate was evaporated to dryness on a rotary evaporator under vacuo. The product was isolated as white foam in quantitative yields.

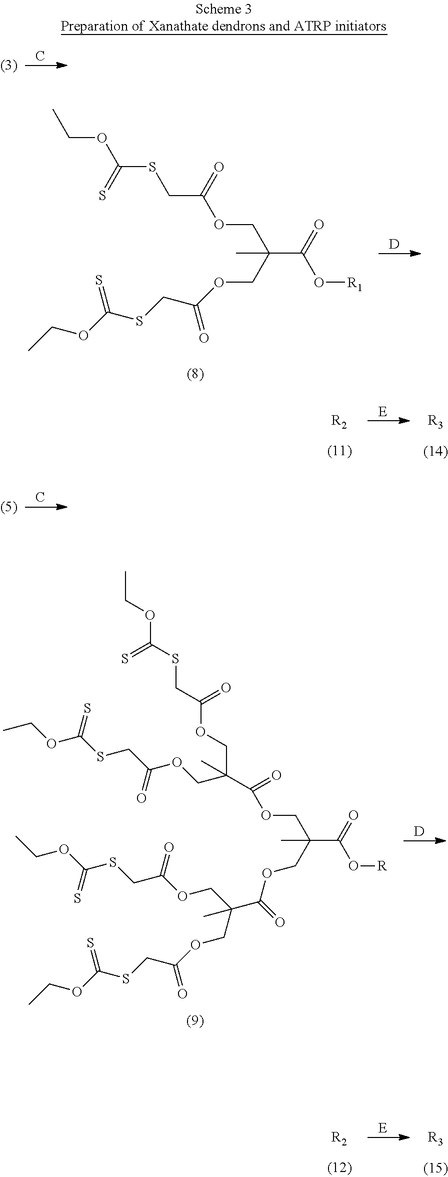

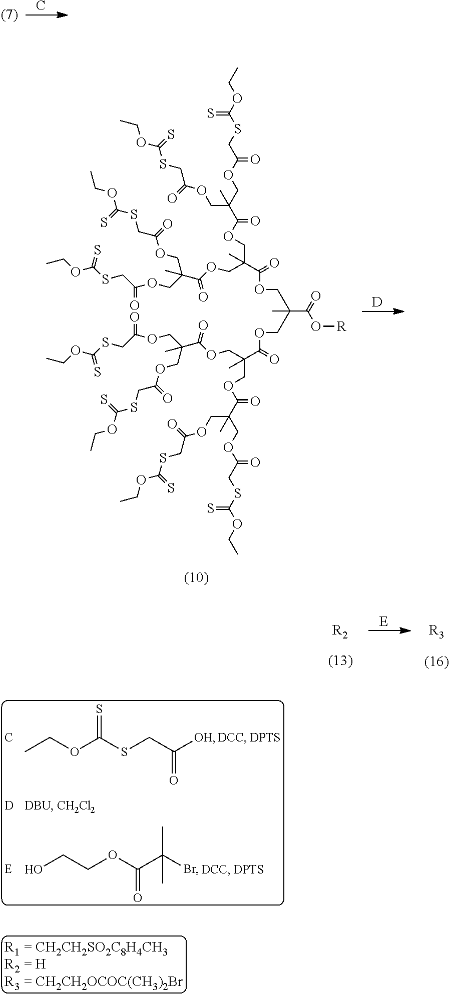

General Procedure for Surface Group Modification to Xanthates (8, 9 an 10)—

To a 500 mL oven-dried round-bottom flask equipped with a magnetic stirrer (under nitrogen atmosphere), the hydroxyl-terminated dendron (generation 0 through to 3), 2-((Ethoxycarbonothioyl)thio)acetic acid 1, and 4-(Dimethylamino)pyridinium 4-toluenesulfonate (DPTS) were all dissolved in the minimum amount of CH₂Cl₂. After the reaction flask was flushed with nitrogen, DCC was added. Stirring at room temperature was continued for 18 h under a nitrogen atmosphere. Once the reaction was complete the DCC-urea was filtered off and washed with a small volume of CH₂Cl₂. The crude product was purified by liquid chromatography on silica gel, eluting with hexane gradually increasing to 40:60 ethyl acetate/hexane to give a yellow viscous oil.

General Procedure for Deprotection of Para-Toluene Sulfonyl Ester (TSe) by DBU (1, 12 and 13)—

To an oven-dried round-bottom flask equipped with a magnetic stirrer, the benzylidene protected dendrimer was dissolved in 50 mL of CH₂Cl₂. 1.4 mL of 1,8-diazabicyclo[5.4.0]undec-7-ene (DBU) was added. The reaction was stirred under a nitrogen atmosphere for 3 hrs and monitored until completion by TLC (60:40 hexane:ethyl acetate). The product was isolated by diluting the mixture with CH₂Cl₂ (100 mL) and washing with 1 M NaHSO₄ (2×100 mL). The organic layer was dried over MgSO4 and evaporated to dryness. The product was then precipitated three times from hexanes. Any residual solvent was removed under high vacuum to yield a viscous oil with typical yields greater than 95%.

General Procedure for Focal Point Modification to an ATRP Initiator by DCC/DPTS Couplings (14, 15 and 16)—

To a 500 mL oven-dried round-bottom flask equipped with a magnetic stirrer (under nitrogen atmosphere), the carboxylic acid focal point xanthate dendron (generation 0 through to 3), 2-hydroxyethyl 2-bromo-2-methylpropanoate, and 4-(Dimethylamino)pyridinium 4-toluenesulfonate (DPTS) were all dissolved in the minimum amount of CH₂Cl₂. After the reaction flask was flushed with nitrogen, DCC was added. Stirring at room temperature was continued for 18 h under a nitrogen atmosphere. Once the reaction was complete the DCC-urea was filtered off and washed with a small volume of CH₂Cl₂. The crude product was purified by liquid chromatography on silica gel, eluting with hexane gradually increasing to 40:60 ethyl acetate/hexane to give a dark yellow viscous oil.

Synthesis of 2—The dendron growth step was carried out as described above, using para-toluene sulfonyl ethanol (10 g, 50 mmol), benzylidene anhydride (42.65 g, 100 mmol, 2 equiv) and DMAP (2.57 g, 21 mmol)) dissolved in 220 mL of dry CH₂Cl₂ and 120 mL of pyridine, and stirred for 16 h at room temperature. Yield: 19.78 g, white foam (98%). ¹H NMR (400 MHz, CDCl₃): δ=0.96 (s, 3H), 2.43 (s, 3H), 3.47 (t, J=6.3 Hz, 2H), 3.60 (d, J=11.6 Hz, 2H) 4.47 (t, J=6.26 Hz, 2H), 4.53 (d, J=11.54 Hz, 2H), 5.43 (s, 1H), 7.33 (m, 5H), 7.41 (m, 2H), 7.81 (d, J=8.42 Hz, 2H). ¹³C NMR (100 MHz, CDCl₃): δ=17.51, 21.64, 42.46, 55.13, 58.20, 73.32, 101.72, 126.15, 128.19, 128.23, 129.01, 130.09, 136.01, 145.11, 149.86, 173.52.

Synthesis of 3—Deprotection of 2 (5.5 g, 13.60 mmol) in 210 mL of CH₂Cl₂:MeOH (1:1, v/v) was carried out as above for 16 h at room temperature under 10 bar H₂ atmosphere. 0.55 g Pd(OH)₂ was used. Yield: 4.3 g, white foam (99%). ¹H NMR (400 MHz, CD₃OD): δ=1.03 (s, 3H), 2.45 (s, 3H), 3.50 (dd, J=42.53, 10.95 Hz, 4H), 3.59 (t, J=5.98 Hz, 2H), 4.39 (t, J=5.85 Hz, 2H), 7.47 (d, 2H), 7.82 (d, 2H). ¹³C NMR (100 MHz, CD₃OD): δ=17.07, 21.61, 51.58, 55.90, 58.93, 65.66, 129.30, 131.22, 137.76, 146.71, 175.89.

Synthesis of 4—The dendron growth step was carried out as described above, using 3 (4.10 g, 12.96 mmol), benzylidene anhydride (16.58 g, 39 mmol, 3 equiv) and DMAP (0.71 g, 5.38 mmol)) all dissolved in 70 mL of dry CH₂Cl₂ and 35 mL of pyridine, and stirred for 16 h at room temperature. Yield: 8.68 g, white foam (94%). ¹H NMR (400 MHz, CDCl₃): δ=0.95 (s, 6H), 1.09 (s, 3H), 2.37 (s, 3H), 3.10 (t, J=5.8 Hz, 2H), 3.60 (d, J=12.45 Hz, 41H) 4.20 (m, 6H), 4.56 (t, J=9 Hz, 4H), 5.42 (s, 2H), 7.30 (m, 8H), 7.39 (m, 4H), 7.68 (d, J=8.43 Hz, 21H). ¹³C NMR (100 MHz, CDCl₃): δ=17.33, 17.72, 21.56, 42.60, 46.70, 54.65, 58.32, 65.20, 73.46, 73.53, 101.63, 126.12, 128.05, 128.16, 128.91, 130.00, 136.29, 137.78, 145.00, 172.00, 173.17. Accurate MS Calc'd for C₃₈H₄₄O₁₂S [M+Na]⁺=747.2451. Found: [M+Na]⁺=742.2426, ES MS: [M+Na]==747.20, [M+K]⁺=763.2.

Synthesis of 5—Deprotection of 4 (7.90 g, 10.90 mmol) in 190 mL of CH₂Cl₂:MeOH (1:1, v/v) was carried out as above for 16 h at room temperature under 10 bar H₂ atmosphere. 0.40 g Pd(OH)₂ was used. Yield: 5.93 g, white foam (99%). ¹H NMR (400 MHz, CD₃OD): δ=1.15 (s, 9H), 2.48 (s, 3H), 3.57-3.69 (m, 10H), 4.11 (dd, J=31.18, 9.37 Hz) 4H), 4.46 (t, J=5.77 Hz, 2H), 7.49 (d, J=8.81 Hz, 2H), 7.85 (d, J=8.39 Hz, 2H). ¹³C NMR (100 MHz, CD₃OD): δ=15.38, 15.94, 19.72, 45.76, 49.91, 53.92, 57.75, 63.95, 64.25, 127.40, 129.41, 136.02, 144.82, 171.81, 173.94. Accurate MS Calc'd for C₂₄H₃₆O₁₂S [M+Na]⁺ m/z=571.1825, [M+Na]⁺ m/z=571.1821. Found ES MS: [M+Na]⁺=571.2, [M+K]⁺=587.2.

Synthesis of 6—The dendron growth step was carried out as described above, using 5 (2.5 g, 4.56 mmol), benzylidene anhydride (11.67 g, 27.36 mmol, 6 equiv) and DMAP (0.35 g, 2.83 mmol)) all dissolved in 46 mL of dry CH₂Cl₂ and 23 mL of pyridine, and stirred for 16 h at room temperature. Yield: 6.23 g, white foam (94%).

¹H NMR (400 MHz, CDCl₃): δ=0.93 (m, 15H), 1.19 (s, 6H), 2.39 (s, 3H), 3.28 (t, J=6.38 Hz, 2H), 3.58 (d, J=11.82 Hz, 8H), 3.94 (dd, J=30.95, 11.33 Hz, 4H), 4.33 (m, 10H), 4.56 (d, J=12 Hz, 8H), 5.40 (s, 4H), 7.30 (m, 14H), 7.39 (m, 8H), 7.74 (d, J=8.52 Hz, 2H). ¹³C NMR (100 MHz, CDCl₃): δ=16.85, 17.66, 21.59, 42.59, 46.30, 46.87, 54.58, 58.22, 65.14, 65.70, 73.44, 73.52, 101.68, 126.20, 128.07, 128.13, 128.88, 130.04, 136.26, 137.82, 144.50, 171.63, 171.83, 173.20. ES MS: [M+Na]⁺=1387.5, [M+K]⁺=1403.5.

Synthesis of 7—Deprotection of 6 (5.80 g, 4.25 mmol) in 200 mL of CH₂Cl₂: MeOH (1:1, v/v) was carried out as above for 16 h at room temperature under 10 bar Hz atmosphere. 0.29 g Pd(OH)₂ was used. Yield: 4.31 g, white foam (99%). ¹H NMR (400 MHz, CD₃OD): δ=1.15 (m, 15H), 1.28 (s, 6H), 2.48 (s, 3H), 3.62 (m, 18H), 4.24 (m, 12H), 4.48 (t, J=6.14 Hz, 2H), 7.49 (d, J=8.10 Hz, 2H), 7.85 (d, J=8.19 Hz, 2H). ES MS: [M+Na]⁺=1035.4, [M+K]=1051.4.

Synthesis of 8—1, 4.65 g (25.80 mmol), and 2.72 g (8.60 mmol) of 3, 1.01 g (3.44 mmol) of DPTS, and 5.86 g (28.38 mmol) of DCC were allowed to react according to the general esterification procedure in 40 mL of dry CH₂Cl₂ for 18 h. The crude product was purified by liquid chromatography on silica gel, eluting with hexane gradually increasing to 40:60 ethyl acetate/hexane to give 6 as a yellow viscous oil 4.6 g (84%). ¹H NMR (400 MHz, CDCl₃): δ=1.16 (s, 3H), 1.42 (t, J=7.15, 6H), 2.46 (s, 3H), 3.44 (t, J=6.3 Hz, 2H), 3.91 (s, 4H), 4.18 (dd, J=31.72, 11.36 Hz, 4H) 4.46 (t, J=6.03 Hz, 2H), 4.64 (q, J=7.12 Hz, 4H), 7.39 (d, J=8.23, 2H), 7.80 (d, J=7.70, 2H). ¹³C NMR (100 MHz, CDCl₃): δ=13.74, 17.56, 21.67, 37.70, 54.97, 58.36, 60.39, 66.21, 70.91, 128.12, 130.18, 136.18, 145.28, 167.33, 171.80, 212.57.

ES MS: [M+Na]⁺=663.0, [M+K]⁺=679.0

Synthesis of 9—1, 9.97 g (55.32 mmol), and 5.06 g (9.22 mmol) of 5, 2.17 g (7.38 mmol) of DPITS, and 12.56 g (60.85 mmol) of DCC were allowed to react according to the general esterification procedure in 170 mL of dry CH₂Cl₂ for 18 h. The crude product was purified by liquid chromatography on silica gel, eluting with hexane gradually increasing to 50:50 ethyl acetate/hexane to give 6 as a orange viscous oil 9.65 g (88%). ¹H NMR (400 MHz, CDCl₃): δ=1.20 (s, 3H), 1.25 (s, 6H), 1.42 (t, J=7.16, 12H), 2.47 (s, 3H), 3.44 (t, J=5.97 Hz, 2H), 3.94 (s, 8H), 4.25 (m, 12H) 4.46 (t, J=5.90 Hz, 2H), 4.64 (q, J=7.01 Hz, 8H), 7.40 (d, J=8.51, 2H), 7.82 (d, J=8.31, 2H).

Synthesis of 10—See the general procedure

Synthesis of 11—The removal of the para-toluene sulfonyl protecting group was carried out as described above, using 8 (4.60 g, 7.18 mmol, 1.0 equiv), and DBU (1.40 mL, 9.33 mmol, 1.3 equiv) dissolved in 80 mL of CH₂Cl₂ and stirred for 3 h. The reaction was monitored using TLC, 40:60 ethyl acetate/hexane. Yield: 3.29 g, orange viscous oil (99%). ¹H NMR (400 MHz, CDCl₃): δ=1.32 (s, 3H), 1.42 (t, J=7.05, 6H), 2.47 (s, 3H), 3.94 (s, 4H), 4.33 (dd, J=39.96, 11.14 Hz, 2H), 4.64 (q, J=7.14 Hz, 4H). ¹³C NMR (100 MHz, CDCl₃): δ==13.74, 17.86, 37.74, 46.06, 66.13, 70.87, 167.45, 177.80, 212.53. ES MS: [M+Na]⁺=481.0

For the synthesis of 12 and 13,—see the general procedure

1.4.7 G1 Morpholine Dendron Initiator (G1 ML Br)

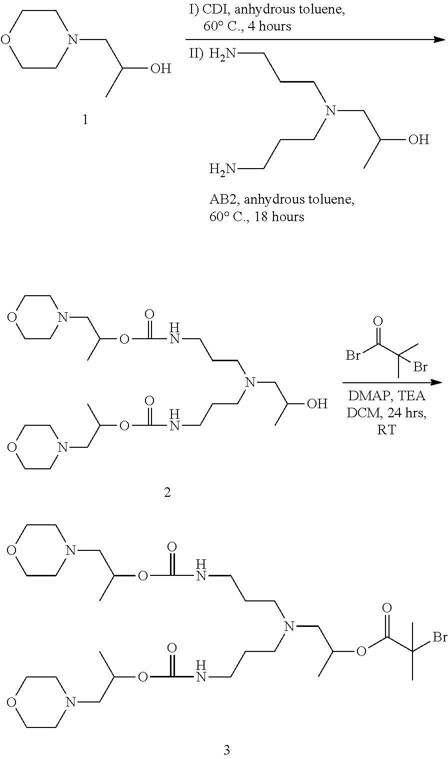

1,1′-Carbonyldiimidazole (6.0994 g, 37.62 mmol) was added to a 2-neck round bottom flask, which was equipped with magnetic stirring, condenser and a N₂ inlet. Anhydrous toluene (60 ml) and N-(2-Hydroxypropyl)morpholine, 1, (5.35 ml, 37.62 mmol) were added and the reaction was heated to 60° C. The AB₂ brancher (3.5603 g, 18.81 mmol) dissolved in anhydrous toluene (6.0 ml) was added after 3 hours of reaction. After a further 16 hours the reaction was stopped, the toluene removed in vacuo. the crude mixture was dissolved in dichloromethane (100 ml) and washed with NaOH solution (pH 14) (3×100 ml). The organic phase was dried over Na₂SO₄ the solvent removed in vacuo and the resulting yellow oil was dried further under high vacuum to give 2, (75%). ¹H NMR (400 MHz, CDCl₃): δ 1.13 (d, 3H), 1.22 (d, 6H), 1.67 (m, 4H), 2.25-2.65 (br m, 18H), 3.22 (m, 4H), 3.68 (m, 8H), 3.79 (m, 1H), 4.98 (m, 2H), 5.29 and 5.40 (br s, NH). ¹³C NMR (100 MHz, CDCl₃): δ 19.30, 20.83, 27.58, 27.76, 39.59, 52.28, 54.39, 62.86, 64.08, 67.36, 67.96, 68.12, 156.73. Calcd.: [M]⁺ m/z=531.36. Found: ES-MS: [M+H]⁺=532.4, [M+Na]⁺⁼=554.4. Found; C, 56.58; H, 9.24; N, 13.23%. C₂₅H₄₉N₅O₇ requires; C, 56.47; H, 9.29; N, 13.17%.

2, (7.546 g, 14.2 mmol) was dissolved in DCM (150 ml) and bubbled with N₂ for 20 minutes. 4-(Dimethylamino)pyridine (DMAP) (86.7 mg, 0.7 mmol) and triethylamine (TEA) (2.37 ml, 17.0 mmol) were added and the reaction vessel was cooled to 0° C. α-Bromoisobutyryl bromide (1.93 ml, 15.6 mmol) was added dropwise, then the reaction was warmed to room temperature for 16 hours. The reaction colour changed from pale yellow to a dark peach colour over this time period. The organic phase was washed with a saturated solution of NaHCO₃ (3×150 ml) and distilled water (3×150 ml), dried over Na₂SO₄ and the solvent removed in vacuo to give a crude brown coloured oil. This was purified by silica column chromatography with a mobile phase of EtOAc:MeOH (4:1), (Rf=0.49) to give a light brown coloured oil, 3, (49%). ¹H NMR (400 MHz, CDCl₃): δ 1.24 (m, 9H), 1.65 (m, 4H), 1.92 (d, 6H), 2.26-2.70 (br m, 18H), 3.20 (m, 4H), 3.69 (m, 8H), 4.98 (m, 2H), 5.06 (m, 1H) 5.36 (br s, NH). ¹³C NMR (100 MHz, CDCl₃): δ. Calcd.: [M]⁺ m/z=679.32. Found: ES-MS: [M+H]⁺=680.3, [M+Na]=702.3. Found; C, 50.87; H, 7.95; N, 10.37%. C₂₉H₅₄N₅O₈Br requires; C, 51.17; H, 8.00; N, 10.29%.

1.4.8 G1 bisMPA Dendron Initiator (G1 MPA Br)

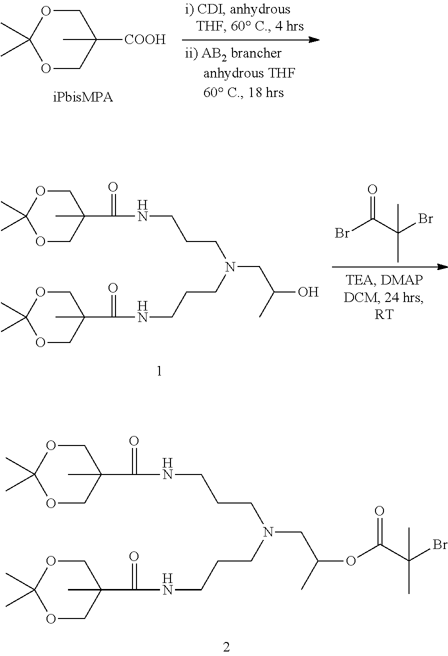

1,1′-Carbonyldiimidazole (9.729 g, 60.0 mmol) was weighed into a 3-neck round bottom flask fitted with a N₂ inlet, magnetic stirrer and condenser. Anhydrous THF (120 ml) was added via double ended needle. The reaction was heated to 60° C. and iPbisMPA (10.4514 g, 60.0 mmol) was added under a positive N₂ flow. Reaction could be observed by the evolution of CO₂ and the reaction became effervescent. To avoid too much effervescence the iPbisMPA was added slowly, approx. 2 g at a time once the effervescence had died down. After 3 hours the reaction mixture was bubbled through with N₂ to ensure any residual CO₂ had been removed from the reaction medium and flask. The AB₂ brancher (5.949 g, 30.0 mmol) was added dropwise in anhydrous THF (20 ml), after a further 18 hours the reaction was stopped and THF removed in vacuo. The crude residue was dissolved in DCM (125 ml) and washed with NaOH solution (pH14) (3×125 ml) and distilled water (125 ml). The organic phase was dried over Na₂SO₄ and the DCM was removed in vacuo then under high vacuum, to give a pale yellow oil, 1, (78%). ¹H NMR (400 MHz, CDCl₃): δ 1.02 (s, 6H), 1.10 (d, 3H), 1.42 (s, 6H), 1.47 (s, 6H), 1.70 (m, 4H), 2.32 (d of d of d, 2H), 2.45 (m, 2H), 2.63 (m, 2H), 3.34 (q, 4H), 3.75 (m, 5H), 3.92 (d, 4H). ¹³C NMR (100 MHz, CDCl₃): δ 18.30, 19.11, 20.38, 27.57, 29.08, 37.87, 40.59, 51.85, 63.00, 63.64, 67.54, 98.93, 175.24. Calcd.: [M]⁺ m/z 501.34. Found: CI-MS: [M+H]⁺⁼502.7. Found; C, 59.86; H, 9.41; N, 8.18%. C₂₅H₄₇N₃O₇ requires; C, 59.86; H, 9.44; N, 8.38%.

G1 MPA OH dendron (5.127 g, 10.2 mmol) was weighed into a round bottom flask and dissolved in DCM (70 ml) and degassed with dry nitrogen for 10 min. DMAP (62 mg, 0.51 mmol) and TEA (1.71 ml, 12.3 mmol) were added, the vessel was maintained under a positive nitrogen flow and cooled to 0° C. α-Bromoisobutyryl bromide (1.38 ml, 11.2 mmol) was added dropwise then was warmed to room temperature for 18 hours. The reaction was a light yellow colour to begin with and changed to a slightly darker yellow over time, no precipitate was observed. The reaction mixture was washed with a saturated NaHCO₃ solution (3×100 ml) and water (3×100 ml), dried over Na₂SO₄ and concentrated in vacuo to give G1 MPA Br, 2, (54%) as a yellow viscous oil. ¹H NMR (400 MHz, CDCl₃): δ 1.04 (s, 6H), 1.24 (d, 3H), 1.42 (s, 6H), 1.46 (s, 6H), 1.67 (m, 4H), 1.91 (s, 6H), 2.40-2.67 (m, 6H), 3.31 (m, 4H), 3.74 (d, 4H), 3.96 (d, 4H), 5.05 (m, 1H). ¹³C NMR (100 MHz, CDCl₃): δ 18.35, 18.43, 27.58, 28.48, 31.16, 37.85, 40.69, 52.09, 56.54, 59.54, 67.44, 67.51, 70.94, 98.74, 171.67, 175.12. Calcd.: [M]⁺ m/z=649.29. Found: ES-MS: [M+H]⁺=650.3. Found; C, 53.61; H, 8.16; N, 6.42%. C₂₉H₅₃BrN₃O₇ requires; C, 53.53; H, 8.06; N, 6.46%.

1.4.9 G1-A Tertiary Amine Dendron Initiator

Synthesis of G1-A Dendron

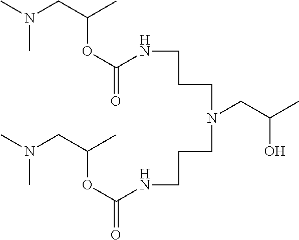 [SMILES: CC(O)CN(CCCNC(=O)OC(C)CN(C)C)CCCNC(=O)OC(C)CN(C)C]

1-dimethylamino-2-propanol (2.4758 g, 24 mmol, 4 eq.) was added to a 100 mL 2 necked round-bottomed flask containing anhydrous toluene (20 mL) and fitted with a reflux condenser, magnetic stirrer and a positive flow of N₂. The solution was stirred at room temperature and CD1 (1.9458 g, 12 mmol, 2 eq.) was added. The mixture was heated to 60° C. with stirring for 6 hours. AB₂ brancher (1.1358 g, 6 mmol, 1 eq.) dissolved in anhydrous toluene (5 mL) was deoxygenated using a N₂ purge for 10 minutes and was added drop wise while the solution was stirred and the temperature was maintained at 60° C.: The reaction was stirred for a further 18 hours at 60° C., and then allowed to cool to room temperature. The solution was concentrated in vacuo, and the remaining oil was dissolved in DCM (30 mL) and washed with 1M NaOH solution (3×30 mL). The solution was dried with anhydrous Na₂SO, filtered and concentrated in vacuo to give G1-A as a viscous liquid. ¹H NMR (400 MHz, CDCl₃) δ 1.25 (m, 9H), 1.64 (m, 3H), 2.05-2.67 (m, 22H), 3.20 (m, 3H), 3.78 (m, 1H), 4.89 (m, 2H). m/z (ES MS) 448.4 [M+H]+, 470.3 [M+Na]⁺.

Synthesis of G1-A Dendron Initiator

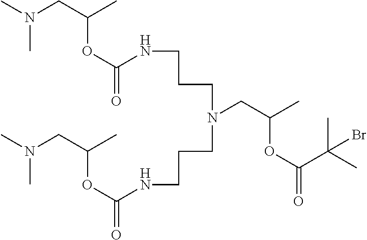 [SMILES: CC(CN(C)C)OC(=O)NCCCN(CCCNC(=O)OC(C)CN(C)C)CC(C)OC(=O)C(C)(C)Br]

G1-A (0.8944 g, 2 mmol, 1 eq.), TEA (0.2833 g, 2.8 mmol, 1.4 eq.) and DMAP (24.43 mg, 0.2 mmol, 0.1 eq.) were added to a 100 mL 2 necked round-bottomed flask containing DCM (40 mL). The flask was deoxygenated under a positive N₂ purge for 10 minutes. α-bromoisobutyryl bromide (0.4828, 0.26 mL, 2.7 mmol, 1.05 eq.) was added drop wise while the solution was stirring in an ice bath under a positive flow of N₂. The reaction mixture was allowed to warm to room temperature and left stirring overnight. The organic phase was washed with saturated sodium hydrogen carbonate (NaHCO₃) solution (3×30 mL). The solution was dried with anhydrous Na₂SO₄, filtered and concentrated in vacuo to give initiator 01-A as a viscous yellow liquid. ¹H NMR (400 MHz, CDCl₃) δ 1.24 (m, 9H), 1.64 (m, 4H), 1.92 (d of d, 8H), 2.05-2.05-2.67 (m, 22H), 3.21 (m, 4H), 4.89 (m, 2H), 5.06 (m, 1H). m/z (ES MS) 596.3 [M+H]+, 617.3 [M+Na]+, 639.2 [M+K]⁺.

1.4.10 G1-D Tertiary Amine Dendron Initiator

Synthesis of G1-D Dendron (HR2-136)

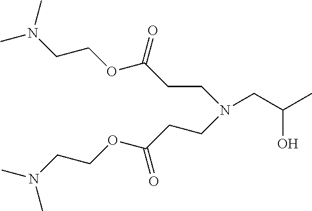 [SMILES: CC(O)CN(CCC(=O)OCCN(C)C)CCC(=O)OCCN(C)C]

2-(Dimethylamino)ethyl acrylate (6.0 g, 42 mmol, 6 eq.) was added to a 50 mL round 2 necked round-bottomed flask containing IPA (12 mL). The flask was deoxygenated under a positive N₂ purge for 10 minutes. 1-amino-2-propanol (0.5246 g, 7.0 mmol, 1 eq.) dissolved in IPA (12 mL) was added drop wise while the solution was stirring in an ice bath under a positive flow of N₂. The final mixture was stirred for a further 10 minutes at 0° C. before being allowed to warm to room temperature and left stirring for 48 hrs. The solvent was removed and the product left to dry in vacuo overnight. ¹H NMR (400 MHz, CDCl₃) δ 1.08 (d, 3H), 2.18-2.62 (m, 22H), 2.69 (m, 2H), 2.89 (m, 2H), 3.77 (m, 1H), 4.16 (m, 4H). m/z (ES MS) 362.3 [M+H]+, 384.3 [M+Na]⁺.

Synthesis of G1-D Dendron Initiator (HR2-143)

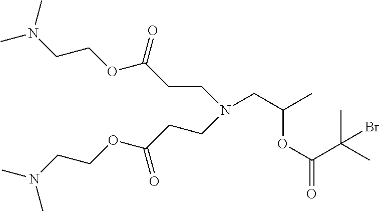 [SMILES: CC(CN(CCC(=O)OCCN(C)C)CCC(=O)OCCN(C)C)OC(=O)C(C)(C)Br]

G1-D dendron (1.1207 g, 10.86 mmol, 1 eq.), TEA (1.5390 g, 15.2 mmol, 1.4 eq.) and DMAP (132.7 mg, 1.086 mmol, 0.1 eq.) were added to a 250 mL 2 necked round-bottomed flask containing DCM (160 mL). The flask was deoxygenated under a positive N₂ purge for 10 minutes. α-bromoisobutyryl bromide (2.622 g, 1.4 mL, 11.4 mmol, 1.05 eq.) was added drop wise while the solution was stirring in an ice bath under a positive flow of N₂. The reaction mixture was allowed to warm to room temperature and left stirring overnight. The organic phase was washed with saturated sodium hydrogen carbonate (NaHCO₃) solution (3×160 mL). The solution was dried with anhydrous Na₂SO₄ and the product left to dry in vacuo overnight. ¹H NMR (400 MHz, CDCl₃) δ 1.22 (d, 3H), 1.89 (m, 6H), 2.24-2.69 (m, 22H), 2.83 (m, 4H), 4.20 (m, 4H), 5.0 (m, 2H). m/z (ES MS) 510.2 [M+H]+, 534.2 [M+Na]⁺.

Synthesis of G2-D dendron (HR2-116)

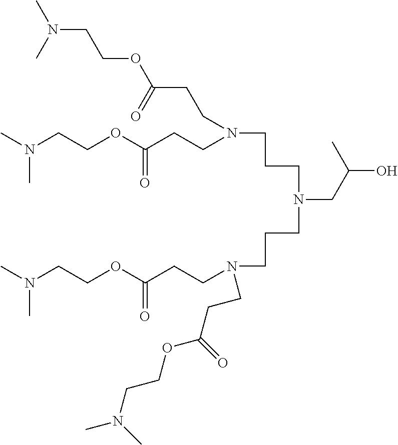 [SMILES: CC(O)CN(CCCN(CCC(=O)OCCN(C)C)CCC(=O)OCCN(C)C)CCCN(CCC(=O)OCCN(C)C)CCC(=O)OCCN(C)C]

2-(Dimethylamino)ethyl acrylate (6.0 g, 42 mmol, 6 eq.) was added to a 50 mL round 2 necked round-bottomed flask containing IPA (12 mL). The flask was 0.1 g deoxygenated under a positive N₂ purge for 10 minutes. Bis(3-aminopropyl)amino)propan-2-ol (1.3221 g, 6.984 mmol, 1 eq.) dissolved in IPA (12 mL) was added drop wise while the solution was stirring in an ice bath under a positive flow of N₂. The final mixture was stirred for a further 10 minutes at 0° C., allowed to warm to room temperature and left stirring for 48 hrs. The solvent was removed and the product left to dry in vacuo overnight. ¹H NMR (400 MHz, CDCl₃) δ 1.13 (d, 3H), 1.67 (m, 4H), 2.26-2.65 (m, 50H), 2.77 (m, 8H), 3.87 (m, 1H), 4.17 (m, 8H). m/z (ES MS) 762.6 [M+H]+, 784.6 [M+Na]⁺.

1.4.11 G2D Tertiary Amine Dendron Initiator

Synthesis of G2-D Dendron Initiator (HR2-12H)

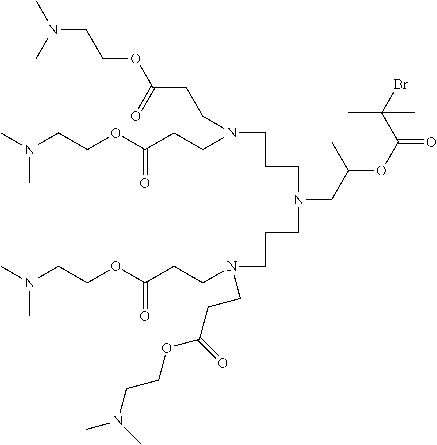 [SMILES: CC(CN(CCCN(CCC(=O)OCCN(C)C)CCC(=O)OCCN(C)C)CCCN(CCC(=O)OCCN(C)C)CCC(=O)OCCN(C)C)OC(=O)C(C)(C)Br]

G2-dendron (5.1431 g, 6.749 mmol, 1 eq.), TEA (0.9561 g, 9.449 mmol, 1.4 eq.) and DMAP (82.5 mg, 0.6749 mmol, 0.1 eq.) were added to a 250 mL 2 necked round-bottomed flask containing DCM (160 mL). The flask was deoxygenated under a positive N₂ purge for 10 minutes. α-bromoisobutyryl bromide (1.629 g, 0.88 mL, 7.087 mmol, 1.05 eq.) was added drop wise while the solution was stirring in an ice bath under a positive flow of N₂. The reaction mixture was allowed to warm to room temperature and left stirring overnight. The organic phase was washed with saturated sodium hydrogen carbonate (NaHCO₃) solution (3×160 mL). The solution was dried with anhydrous Na₂SO₄ and the product left to dry in vacuo overnight. ¹H NMR (400 MHz, CDCl₃) δ 1.26 (d, 3H), 1.56 (m, 4H), 1.91 (m, 6H), 2.22-2.67 (m, 50H), 2.76 (m, 8H), 4.19 (m, 8H), 5.04 (m, 1H). m/z (ES MS) 912.5 [M+H]+, 934.5 [M+Na]+, 950.5 [M+K]⁺.

### 2. Polydendrons—100% Dendron Initiated Branched Polymers

2.1 HPMA (Hydrophobic Polymer Core)

2.1.1 Hydrophobic Dendron Initiators

2.1.1.1 Aromatic Dendrons G1 and G2 DBOP Br

In a typical experiment, G1 DBOP Br (0.291 g, 0.69 mmol) or G2 DBOP Br (0.648 g, 0.69 mmol) and HPMA (targeted DP=50) (5.0 g, 34.7 mmol) were weighed into a round bottom flask. EGDMA (105 μl, 0.55 mmol) was added and the flask was equipped with magnetic stirrer bar, sealed and degassed by bubbling with N₂ for 20 minutes and maintained under N₂ at 30° C. Anhydrous methanol was degassed separately and subsequently added to the monomer/initiator/brancher mixture via syringe to give a 50% v/v mixture with respect to the monomer. The catalytic system; Cu(I)Cl (0.069 g, 0.69 mmol) and 2,2′-bipyridyl (bpy) (0.217 g, 1.39 mmol), were added under a positive nitrogen flow in order to initiate the reaction. The polymerisations were stopped when conversions had reached over 98%. The polymerisations were stopped by diluting with a large excess of tetrahydrofuran (THF), which caused a colour change from dark brown to a bright green colour. The catalytic system was removed using Dowex® Marathon™ MSC (hydrogen form) ion exchange resin beads and basic alumina. The resulting polymer was isolated by precipitation from the minimum amount of THF into cold hexane. The [initiator]:[CuCl]:[bpy] molar ratios in all polymerizations were 1:1:2. Other DPs targeted were DP20 and DP100 with both G1 and G2 DBOP initiators.

2.1.1.2 ^{t}BOC dendrons G1 ^{t}BOC Br

The G1 ^{t}BOC Dendron initiator (100 mg, 0.186 mmol) was added to a 25 mL round bottom flask equipped with a magnetic stirrer bar, followed by the addition of 2,2-bipyridyl (58.1 mg, 0.372 mmol), EGDMA (35.1 mg, 0.177 mmol) and HPMA (1.34 g, 9.28 mmol). The reaction mixture was bubbled with N₂ for 15 minutes. Degassed anhydrous methanol (3.45 mL) was added to the flask, and its contents stirred and bubbled with N₂ for a further 5 minutes. Copper (I) chloride (18.4 mg, 0.186 mmol) was quickly weighed out and added to the flask, instantly forming a brown coloured mixture, which was stirred and bubbled with N₂ for a further 5 minutes. A N₂ pressure was then built up within the flask, then N₂ inlet removed, and the flask stirred for 24 hours at 40° C. Once the polymerisation was complete, THF was added to the reaction flask to poison the Cu (I) catalyst, forming a green coloured solution. The solution was passed through an alumina (neutral) column to remove the catalytic system, concentrated in vacuo, and precipitated into hexane. The supernatant was decanted off and the remaining white solid dried overnight in a vac-oven.

2.1.1.3 Xanthate Dendron Xant-G1

Xant-G1 initiator (578.0 mg, 0.868 mmol), BIPY (272.2 mg, 1.743 mmol), HPMA (6.3 g, 43.6 mmol,) and EGDMA (146.8 mg, 0.741 mmol) was transferred to 25 mL round-bottomed flask equipped with stirrer bar and septa cap. The flask was deoxygenated using nitrogen. Separately deoxygenated MeOH (12.9 mL, 38% w/v based on HPMA) added via syringe. Once all reactants had dissolved, nitrogen was bubbled through solution for 5 mins. Cu (I) Cl (86.3 mg, 0.868 mmol,) quickly measured out and added to round-bottomed flask. Reaction mixture went from clear solution to deep red/brown. Nitrogen was bubbled through solution for an additional 10 mins. The reaction was then left to stir overnight under nitrogen. Reaction mixture forms a deep red/brown viscous liquid on completion. THF (20 mL) added to kill reaction. Once solution turned a bright green colour, solution passed through a short alumina column to remove copper catalyst, yielding a translucent pale green solution. Solvent removed and resulting oily liquid precipitated into cold hexane (approx. 50 mL, cooled in dry ice bath). The resulting pale green crystals were filtered off and washed with cold hexane. The sample was placed in a vacuum oven to remove any residual solvent.

2.1.2 Hydrophilic Dendrons

2.1.2.1 G1-A Tertiary Amine Initiator

In a typical synthesis, targeting a number average degree of polymerisation (DPₙ)=50 monomer units (poly(HPMA)₅₀; n_{DEAEMA}/n_{Initiator}: 50), bpy (173.3 mg, 1.1096 mmol, 2 eq.), HPMA (4 g, 27.7 mmol, 50 eq.), EGDMA (77.0 ng, 0.3883 mmol, 0.7 eq) and isopropanol (IPA) (38.9% v/v based on HPMA) were placed into a 25 mL round-bottomed flask. The solution was stirred and deoxygenated using a nitrogen (N₂) purge for 15 minutes. Cu(ᵢ)Cl (54.9 mg, 0.5548 mmol, 1 eq.) was added to the flask and left to purge for a further 5 minutes. G1-A dendron initiator (0.3310 g, 0.5548 mmol, 1 eq.) was added to the flask under a positive flow of N₂, and the solution was left to polymerise at 40° C. Reactions were terminated when >99% conversion was reached, as judged by ¹H NMR, by exposure to oxygen and addition of THF. The catalyst residues were removed by passing the mixture over a basic alumina column. THF was removed under vacuum to concentrate the sample before precipitation into hexane and drying in the vacuum oven overnight.

2.1.2.2 G1 Morpholine Initiator (G1 ML Br)

G1 ML Br (0.378 g, 0.55 mmol) and HPMA (4.0 g, 27.7 mmol) were weighed into a round bottom flask. EGDMA (73.2 μl, 0.39 mmol) was added and the flask was equipped with magnetic stirrer bar, sealed and degassed by bubbling with N₂ for 20 minutes and maintained under N₂ at 30° C. Isopropanol was degassed separately and subsequently added to the monomer/initiator/brancher mixture via syringe to give a 50 wt/wt % mixture with respect to the monomer. The catalytic system; Cu(I)CI (0.055 g, 0.55 mmol) and 2,2′-bipyridyl (bpy) (0.173 g, 1.1 mmol), were added under a positive nitrogen flow in order to initiate the reaction. The polymerisations were stopped when conversions had reached over 98%. The polymerisations were stopped by diluting with a large excess of tetrahydrofuran (THF), which caused a colour change from dark brown to a bright green colour. The catalytic system was removed using Dowex® Marathon™ MSC (hydrogen form) ion exchange resin beads and basic alumina. The resulting polymer was isolated by precipitation from the minimum amount of THF into cold hexane. The [initiator]:[CuCl]:[bpy] molar ratios in all polymerizations were 1:1:2

2.1.2.3 G1 bisMPA Initiator (G1 MPA Br)

G1 MPA Br (0.451 g, 0.69 mmol) and HPMA (5.0 g, 34.7 mmol) were weighed into a round bottom flask. EGDMA (105 μl, 0.55 mmol) was added and the flask was equipped with magnetic stirrer bar, sealed and degassed by bubbling with N₂ for 20 minutes and maintained under N₂ at 30° C. Isopropanol was degassed separately and subsequently added to the monomer/initiator/brancher mixture via syringe to give a 50 wt/wt % mixture with respect to the monomer. The catalytic system; Cu(I)Cl (0.0687 g, 0.69 mmol) and 2,2′-bipyridyl (bpy) (0.217 g, 1.39 mmol), were added under a positive nitrogen flow in order to initiate the reaction. The polymerisations were stopped when conversions had reached over 98%. The polymerisations were stopped by diluting with a large excess of tetrahydrofuran (THF), which caused a colour change from dark brown to a bright green colour. The catalytic system was removed using Dowex® Marathon™ MSC (hydrogen form) ion exchange resin beads and basic alumina. The resulting polymer was isolated by precipitation from the minimum amount of THF into cold hexane. The [initiator]:[CuCl]:[bpy] molar ratios in all polymerizations were 1:1:2.

2.2 tBuMA (Hydrophobic Core)

2.2.1 G1-A Tertiary Amine Dendron Initiator

In a typical synthesis, targeting a number average degree of polymerisation (DPₙ)=50 monomer units (poly(tBuMA)₅₀; n_{DEAEMA}/n_{Initiator}: 50), bpy (175.7 mg, 1.1252 mmol, 2 eq.), tBuMA (4 g, 28.13 mmol, 50 eq.), EGDMA (105.9 mg, 0.5345 mmol, 0.95 eq) and aqueous isopropanol (7.5% water by volume) (33.3% v/v based on tBuMA) were placed into a 25 mL round-bottomed flask. The solution was stirred and deoxygenated using a nitrogen (N₂) purge for 15 minutes. Cu(ᵢ)Cl (55.7 mg, 0.5626 mmol, 1 eq.) was added to the flask and left to purge for a further 5 minutes. G1-A dendron initiator (0.3356 g, 0.5626 mmol, 1 eq.) was added to the flask under a positive flow of N₂, and the solution was left to polymerise at 40° C. Reactions were terminated when >99% conversion was reached, as judged by ¹H NMR, by exposure to oxygen and addition of THF. The catalyst residues were removed by passing the mixture over a basic alumina column. THF was removed under vacuum to concentrate the sample before precipitation into hexane and drying in the vacuum oven overnight.

2.3 DEAEMA (Hydrophobic Core at Neutral/High pH, Hydrophilic at Low pH)

2.3.1 G1-A Tertiary amine dendron initiator

In a typical synthesis, targeting a number average degree of polymerisation (DPₙ)=50 monomer units (poly(DEAEMA)₅₀; n_{DEAEMA}/n_{Initiator}: 50), bpy (134.9 mg, 0.8637 mmol, 2 eq.), DEAEMA (4 g, 21.59 mmol, 50 eq.), EGDMA (77.0 mg, 0.3886 mmol, 0.9 eq) and IPA³⁷ (38.9% v/v based on DEAEMA) were placed into a 25 mL round-bottomed flask. The solution was stirred and deoxygenated using a N₂ purge for 15 minutes. Cu(ᵢ)Cl (42.8 mg, 0.4318 mmol, 1 eq.) was added to the flask and left to purge for a further 5 minutes. G1-A dendron initiator (0.2576 g, 0.4318 mmol, 1 eq.) was added to the flask under a positive flow of N₂, and the solution was left to polymerise at 40° C. Reactions were terminated when >99% conversion was reached, as judged by ¹H NMR, by exposure to oxygen and addition of acetone. The catalyst residues were removed by passing the mixture over a basic alumina column. Acetone was removed under vacuum to concentrate the sample before precipitation into cold petroleum ether (40° C.-60° C.). The polymerisation conditions and procedure is identical to those described for linear polymers above and drying in the vacuum oven overnight.

2.3.2 G0-D Tertiary Amine Dendron Initiator

In a typical synthesis, targeting a number average degree of polymerisation (DPₙ)=50 monomer units (poly(DEAEMA)₅₀; n_{DEAEMA}/n_{Initiator}: 50), bpy (134.9 mg, 0.8637 mmol, 2 eq.), DEAEMA (4 g, 21.59 mmol, 50 eq.), EGDMA (77.0 mg, 0.3886 mmol, 0.9 eq) and IPA³⁷ (38.9/% v/v based on DEAEMA) were placed into a 25 mL round-bottomed flask. The solution was stirred and deoxygenated using a N₂ purge for 15 minutes. Cu(ᵢ)Cl (42.8 mg, 0.4318 mmol, 1 eq.) was added to the flask and left to purge for a further 5 minutes. G0-D dendron initiator (0.1089 g, 0.4318 mmol, 1 eq.) was added to the flask under a positive flow of N₂, and the solution was left to polymerise at 40° C. Reactions were terminated when >99% conversion was reached, as judged by ¹H NMR, by exposure to oxygen and addition of acetone. The catalyst residues were removed by passing the mixture over a basic alumina column. Acetone was removed under vacuum to concentrate the sample before precipitation into cold petroleum ether (40° C.-60° C.) and drying in the vacuum oven overnight. The polymerisation conditions and procedure is identical to those described for linear polymers above.

2.3.3 G1-D Tertiary Amine Dendron Initiator

In a typical synthesis, targeting a number average degree of polymerisation (DPₙ)=50 monomer units (poly(DEAEMA)₅₀; n_{DEAEMA}/n_{Initiator}: 50), bpy (134.9 mg, 0.8637 mmol, 2 eq.), DEAEMA (4 g, 21.59 mmol, 50 eq.), EGDMA (77.0 mg, 0.3886 mmol, 0.9 eq) and IPA³⁷ (38.9% v/v based on DEAEMA) were placed into a 25 mL round-bottomed flask. The solution was stirred and deoxygenated using a N₂ purge for 15 minutes. Cu(ᵢ)Cl (42.8 mg, 0.4318 mmol, 1 eq.) was added to the flask and left to purge for a further 5 minutes. G1-D dendron initiator (0.2204 g, 0.4318 mmol, 1 eq.) was added to the flask under a positive flow of N₂, and the solution was left to polymerise at 40° C. Reactions were terminated when >99% conversion was reached, as judged by ¹H NMR, by exposure to oxygen and addition of acetone. The catalyst residues were removed by passing the mixture over a basic alumina column. Acetone was removed under vacuum to concentrate the sample before precipitation into cold petroleum ether (40° C.-60° C.) and drying in the vacuum oven overnight. The polymerisation conditions and procedure is identical to those described for linear polymers above.

2.3.4 G2-D Tertiary Amine Dendron Initiator

In a typical synthesis, targeting a number average degree of polymerisation (DPₙ)=50 monomer units (poly(DEAEMA)₅₀; n_{DEAEMA}/n_{Initiator}: 50), bpy (134.9 mg, 0.8637 mmol, 2 eq.), DEAEMA (4 g, 21.59 mmol, 50 eq.), EGDMA (77.0 mg, 0.3886 mmol, 0.9 eq) and IPA³⁷ (38.9% v/v based on DEAEMA) were placed into a 25 mL round-bottomed flask. The solution was stirred and deoxygenated using a N₂ purge for 15 minutes. Cu(ᵢ)Cl (42.8 mg, 0.4318 mmol, 1 eq.) was added to the flask and left to purge for a further 5 minutes. G2-D dendron initiator (0.3934 g, 0.4318 mmol, 1 eq.) was added to the flask under a positive flow of N₂, and the solution was left to polymerise at 40° C. Reactions were terminated when >99% conversion was reached, as judged by ¹H NMR, by exposure to oxygen and addition of acetone. The catalyst residues were removed by passing the mixture over a basic alumina column. Acetone was removed under vacuum to concentrate the sample before precipitation into cold petroleum ether (40° C.-60° C.) and drying in the vacuum oven overnight. The polymerisation conditions and procedure is identical to those described for linear polymers above.

2.4 OEGMA (Hydroplilic Core)

2.4.1 G1-A Tertiary Amine Dendron Initiator

In a typical synthesis, targeting a number average degree of polymerisation (DPₙ)=50 monomer units (poly(OEGMA)₅₀; n_{DEAEMA}/n_{Initiator}: 50), bpy (83.3 mg, 0.5333 mmol, 2 eq.), OEGMA (4 g, 13.3 mmol, 50 eq.), EGDMA (50.2 mg, 0.2533 mmol, 0.95 eq) and aqueous isopropanol (7.5% water by volume) (33.3% v/v based on OEGMA) were placed into a 25 mL round-bottomed flask. The solution was stirred and deoxygenated using a nitrogen (N₂) purge for 15 minutes. Cu(ᵢ)Cl (26.4 mg, 0.2667 mmol, 1 eq.) was added to the flask and left to purge for a further 5 minutes. G1-A dendron initiator (0.1591 g, 0.2667 mmol, 1 eq.) was added to the flask under a positive flow of N₂, and the solution was left to polymerise at 40° C. Reactions were terminated when >99% conversion was reached, as judged by ¹H NMR, by exposure to oxygen and addition of THF. The catalyst residues were removed by passing the mixture over a basic alumina column. THF was removed under vacuum to concentrate the sample before precipitation into cold hexane and drying in the vacuum oven overnight.

2.5 Copolymer Synthesis

2.5.1 G2-D Tertiary Amine Initiator, pDEAEMA₅₀-b-ptBuMA₆₅-st EGDMA_{0.9}

In a typical synthesis, targeting a number average degree of polymerisation (DPₙ)=50 monomer units (poly(DEAEMA)₅₀; n_{DEAEMA}/n_{Initiator}: 50), bpy (134.9 mg, 0.8637 mmol, 2 eq.), DEAEMA (4 g, 21.59 mmol, 50 eq.) and isopropanol (IPA) (37.7% v/v based on DEAEMA) were placed into a 50 mL round-bottomed flask. The solution was stirred and deoxygenated using a nitrogen (N₂) purge for 15 minutes. Cu(ᵢ)CI (42.8 mg, 0.4318 mmol, 1 eq.) was added to the flask and left to purge for a further 5 minutes. G2-D dendron initiator (0.3934 g, 0.4318 mmol, 1 eq.) was added to the flask under a positive flow of N₂, and the solution was left to polymerise at 40° C. In another 25 mL round-bottomed flask, bpy (134.9 mg, 0.8637 mmol), tBuMA (4.0 g, 28.1 mmol, 65 eq.), EGDMA (77.0 mg, 0.3886 mmol, 0.9 eq) and aqueous isopropanol (23.8% v/v based on tBuMA) were added. The solution was stirred and deoxygenated using a nitrogen (N₂) purge for 15 minutes. Cu(ᵢ)Cl (42.8 mg, 0.4318 mmol, 1 eq.) was added to the flask and left to purge for a further 5 minutes. After the conversion of DEAEMA reached around 85%, the mixture from the second flask was added into the first flask rapidly using a syringe and taking care not to admit any air into the vessel. A sample was taken immediately after the addition of the tBuMA monomer solution for ¹H NMR analysis. The block copolymerization reaction was carried out at ambient temperature and samples were taken periodically from the reaction mixture for ¹H NMR analysis. Reactions were terminated when >99% conversion was reached, as judged by ¹H NMR, by exposure to oxygen and addition of acetone. The catalyst residues were removed by passing the mixture over a basic alumina column. Acetone was removed under vacuum to concentrate the sample before precipitation into cold petroleum ether (40° C.-60° C.) and drying in the vacuum oven overnight.

TABLE 1
100% Dendron initiated polydendrons

| | Initiator | Polymer | EGDMA | | | |
|---|---|---|---|---|---|---|
| Generation | Functionality | Core | (mol %) | Mn (gmol⁻¹) | Mw (gmol⁻¹) | PDI |
| G1 | DBOP | pHPMA20 | 0.8 | 52 800 | 545 000 | 10.32 |
| G1 | DBOP | pHPMA50 | 0.8 | 47 200 | 1 169 000 | 24.74 |
| G1 | DBOP | pHPMA100 | 0.8 | 69 300 | 1 354 500 | 19.54 |
| G2 | DBOP | pHPMA20 | 0.8 | 153 000 | 1 565 000 | 10.23 |
| G2 | DBOP | pHPMA50 | 0.8 | 59978 | 739440 | 12.33 |
| G2 | DBOP | pHPMA100 | 0.8 | 164 200 | 2 227 500 | 13.58 |
| G1 | tBOC | pHPMA50 | 0.95 | 12282 | 45539 | 3.71 |
| G1 | Xanthate | pHPMA50 | 0.85 | 63800 | 1070000 | 15 |
| G1 | Morpholine | pHPMA50 | 0.7 | 76687 | 454746 | 5.93 |
| G1 | bisMPA | pHPMA50 | 0.8 | 77745 | 436461 | 5.61 |
| G1-A | t-amine | pHPMA50 | 0.7 | 661180 | 966552 | 1.50 |
| G1-A | t-amine | ptBuMA50 | 0.95 | 150264 | 284002 | 1.90 |
| G1-A | t-amine | pDEAEMA50 | 0.9 | 201497 | 244622 | 1.20 |
| G1-A | t-amine | pOEGMA50 | 0.95 | 97082 | 216813 | 2.20 |
| G0-D | t-amine | pDEAEMA50 | 0.9 | | | |
| G1-D | t-amine | pDEAEMA50 | 0.9 | | | |
| G2-D | t-amine | pDEAEMA50 | 0.9 | 125652 | 302557 | 2.40 |
| G2-D | t-amine | pDEAEMA50- | 0.9 | 129737 | 374192 | 2.90 |

### 3. Mixed Initiator Systems

3.1 Mixed dendrons

3.1.1 G1 and G2 tBOC Initiated pHPMA Core

The G1 ^{t}BOC Dendron initiator (67.9 mg, 0.126 mmol) and G2 ^{t}BOC Dendron initiator (63.1 mg, 0.054 mmol) was added to a 25 mL round bottom flask equipped with a magnetic stirrer bar, followed by the addition of 2,2-bipyridyl (56.2 mg, 0.360 mmol), EGDMA (28.5 mg, 0.144 mmol) and HPMA (1.3 g, 9.0 mmol). The reaction mixture was bubbled with N₂ for 15 minutes. Degassed anhydrous methanol (3.3 mL) was added to the flask, and its contents stirred and bubbled with N₂ for a further 5 minutes. Copper (I) chloride (17.8 mg, 0.180 mmol) was quickly weighed out and added to the flask, instantly forming a brown coloured mixture, which was stirred and bubbled with N₂ for a further 5 minutes. A N₂ pressure was built up within the flask, then N₂ inlet then removed, and the flask stirred for 24 hours at 40° C. Once the polymerisation was complete, THF was added to the reaction flask to poison the Cu (I) catalyst, forming a green coloured solution. The solution was passed through an alumina (neutral) column to remove the catalytic system, concentrated in vacuo, and precipitated into hexane. The supernatant was decanted off and the remaining white solid dried overnight in a vac-oven.

3.2 Mixed Dendron with Non-Dendron Initiator

3.2.1 G2 DBOP Br and 750 PEG initiated pHPMA core

In a typical reaction, G2 DBOP Br (0.259 g, 0.28 mmol) and 750 PEG initiator (0.250 g, 0.28 mmol) (for a targeted ratio of G2 dendron:750 PEG of 50:50 mol %) were weighed into a round bottom flask, followed by HPMA (4.0 g, 27.7 mmol). EGDMA (84 μl, 0.44 mmol) was added and the flask was equipped with magnetic stirrer bar, sealed and degassed by bubbling with N₂ for 20 minutes and maintained under N₂ at 30° C. Anhydrous methanol was degassed separately and subsequently added to the monomer/initiator/brancher mixture via syringe to give a 50 wt/wt % mixture with respect to the monomer. The catalytic system; Cu(I)Cl (0.055 g, 0.55 mmol) and 2,2′-bipyridyl (bpy) (0.173 g, 1.1 mmol), were added under a positive nitrogen flow in order to initiate the reaction. The polymerisations were stopped when conversions had reached over 98%. The polymerisations were stopped by diluting with a large excess of tetrahydrofuran (THF), which caused a colour change from dark brown to a bright green colour. The catalytic system was removed using Dowex® Marathon™ MSC (hydrogen form) ion exchange resin beads and basic alumina. The resulting polymer was isolated by precipitation from the minimum amount of THF into cold hexane. The [initiator]:[CuCl]:[bpy] molar ratios in all polymerizations were 1:1:2.

3.2.2 G2 DBOP Br and 2K PEG Initiated pHPMA Core

In a typical reaction, G2 DBOP Br (0.324 g, 0.35 mmol) and 2K PEG initiator (0.745 g, 0.35 mmol) (for a targeted ratio of G2 dendron:750 PEG of 50:50 mol %) were weighed into a round bottom flask, followed by HPMA (5.0 g, 34.7 mmol). EGDMA (112 μl, 0.59 mmol) was added and the flask was equipped with magnetic stirrer bar, sealed and degassed by bubbling with N₂ for 20 minutes and maintained under N₂ at 30° C. Anhydrous methanol was degassed separately and subsequently added to the monomer/initiator/brancher mixture via syringe to give a 50% v/v mixture with respect to the monomer. The catalytic system; Cu(I)Cl (0.069 g, 0.69 mmol) and 2,2′-bipyridyl (bpy) (0.217 g, 1.39 mmol), were added under a positive nitrogen flow in order to initiate the reaction. The polymerisations were stopped when conversions had reached over 98%. The polymerisations were stopped by diluting with a large excess of tetrahydrofuran (THF), which caused a colour change from dark brown to a bright green colour. The catalytic system was removed using Dowex® Marathon™ MSC (hydrogen form) ion exchange resin beads and basic alumina. The resulting polymer was isolated by precipitation from the minimum amount of THF into cold hexane. The [initiator]:[CuCl]:[bpy] molar ratios in all polymerizations were 1:1:2.

3.2.3 G1 tBOC Dendron and Lactose Initiated pHPMA Core

The G1 ^{t}BOC Dendron initiator (48.5 mg, 0.09 mmol) and Lactose ATRP initiator (70.7 mg, 0.09 mmol) was added to a 25 mL round bottom flask equipped with a magnetic stirrer bar, followed by the addition of 2,2-bipyridyl (56.2 mg, 0.360 mmol), EGDMA (28.5 mg, 0.144 mmol) and HPMA (1.3 g, 9.0 mmol). The reaction mixture was bubbled with N₂ for 15 minutes. Degassed anhydrous methanol (3.3 mL) was added to the flask, and its contents stirred and bubbled with N₂ for a further 5 minutes. Copper (I) chloride (17.8 mg, 0.180 mmol) was quickly weighed out and added to the flask, instantly forming a brown coloured mixture, which was stirred and bubbled with N₂ for a further 5 minutes. A N₂ pressure was built up within the flask, then N₂ inlet then removed, and the flask stirred for 24 hours at 40° C. Once the polymerisation was complete, THF was added to the reaction flask to poison the Cu (I) catalyst, forming a green coloured solution. The solution was passed through an alumina (neutral) column to remove the catalytic system, concentrated in vacuo, and precipitated into hexane. The supernatant was decanted off, and the remaining white solid dried overnight in a vac-oven.

3.2.4 G1 tBOC Dendron and Bifunctional Initiator pHPMA Dumbbell

The G1 ^{t}BOC Dendron initiator (181 mg, 0.336 mmol) and bi-functional initiator (36.6 mg, 0.084 mmol) was added to a 25 mL round bottom flask equipped with a magnetic stirrer bar, followed by the addition of 2,2-bipyridyl (157.4 mg, 1.01 mmol), EGDMA (79.1 mg, 0.399 mmol) and HPMA (3.63 g, 25.2 mmol). The reaction mixture was then bubbled with N₂ for 15 minutes. Degassed anhydrous methanol (10 mL) was added to the flask, and its contents stirred and bubbled with N₂ for a further 5 minutes. Copper (I) chloride (49.9 mg, 0.504 mmol) was quickly weighed out and added to the flask, instantly forming a brown coloured mixture, which was stirred and bubbled with N₂ for a further 5 minutes. A N₂ pressure was built up within the flask, then N₂ inlet then removed, and the flask stirred for 24 hours at 40° C. Once the polymerisation was complete, THF was added to the reaction flask to poison the Cu (I) catalyst, forming a green coloured solution. The solution was passed through an alumina (neutral) column to remove the catalytic system, concentrated in vacuo, and precipitated into hexane. The supernatant was decanted off, and the remaining white solid dried overnight in a vac-oven.

3.2.5 G2 tBOC Dendron and Bifunctional Initiator pHPMA Dumbbell Core

The G2 ^{t}BOC Dendron initiator (197 mg, 0.168 mmol) and bi-functional initiator (18.3 mg, 0.042 mmol) was added to a 25 mL round bottom flask equipped with a magnetic stirrer bar, followed by the addition of 2,2-bipyridyl (78.7 mg, 0.504 mmol), EGDMA (33.3 mg, 0.168 mmol) and HPMA (3.63 g, 12.6 mmol). The reaction mixture was bubbled with N₂ for 15 minutes. Degassed anhydrous methanol (4.65 mL) was added to the flask, and its contents stirred and bubbled with N₂ for a further 5 minutes. Copper (I) chloride (24.9 mg, 0.252 mmol) was quickly weighed out and added to the flask, instantly forming a brown coloured mixture, which was stirred and bubbled with N₂ for a further 5 minutes. A N₂ pressure was built up within the flask, then N₂ inlet then removed, and the flask stirred for 24 hours at 40° C. Once the polymerisation was complete, THF was added to the reaction flask to poison the Cu (I) catalyst, forming a green coloured solution. The solution was passed through an alumina (neutral) column to remove the catalytic system, concentrated in vacuo, and precipitated into hexane. The supernatant was decanted off, and the remaining white solid dried overnight in a vac-oven.

TABLE 2
Mixed initiator polydendrons

| | | Polymer | EGDMA | | | |
|---|---|---|---|---|---|---|
| Initiator 1 | Initiator 2 | Core | (mol %) | Mn (gmol⁻¹) | Mw (gmol⁻¹) | PDI |
| G1 tBOC | G2 tBOC | pHPMA50 | 0.8 | 61500 | 153500 | 2.49 |
| G1 tBOC | Lactose | pHPMA50 | 0.8 | 102000 | 216000 | 2.11 |
| G1 tBOC | bifunctional | pHPMA50 | 0.95 | 47000 | 227000 | 4.83 |
| G2 tBOC | bifunctional | pHPMA50 | 0.8 | 177500 | 555500 | 3.13 |
| G2 DBOP | 750 PEG | | | | | |
| 100 | 0 | pHPMA50 | 0.8 | 90 500 | 1 304 000 | 9.67 |
| 90 | 10 | pHPMA50 | 0.8 | 68457 | 1495000 | 21.84 |
| 75 | 25 | pHPMA50 | 0.8 | 52431 | 987762 | 18.88 |
| 50 | 50 | pHPMA50 | 0.8 | 39447 | 480638 | 12.19 |
| 25 | 75 | pHPMA50 | 0.8 | 36157 | 315320 | 8.73 |
| 10 | 90 | pHPMA50 | 0.8 | 37672 | 286049 | 7.61 |
| 0 | 100 | pHPMA50 | 0.8 | 68133 | 296179 | 4.35 |
| 25 | 75 | pHPMA50 | 0.9 | 60738 | 675119 | 11.13 |
| 0 | 100 | pHPMA50 | 0.95 | 74740 | 642728 | 8.60 |
| G2 DBOP | 2K PEG | | | | | |
| 100 | 0 | pHPMA50 | 0.8 | 193576 | 2225000 | 11.49 |
| 90 | 10 | pHPMA50 | 0.8 | 348067 | 2464000 | 7.08 |
| 75 | 25 | pHPMA50 | 0.8 | 55050 | 1067000 | 19.38 |
| 50 | 50 | PHPMA50 | 0.85 | 29372 | 709209 | 24.15 |
| 25 | 75 | pHPMA50 | 0.95 | 141272 | 1862000 | 13.18 |
| 10 | 90 | pHPMA50 | 0.95 | 40195 | 795274 | 19.79 |
| 0 | 100 | pHPMA50 | 0.95 | 32246 | 476990 | 14.79 |
| 50 | 50 | pHPMA100 | 0.8 | 79448 | 516794 | 6.51 |

### 4. Nanoprecpitation of Polydendrons

4.1 Nanoparticle Formation (Slow Addition)—HR Method

In a typical procedure, 10 mg of sample was completely dissolved in 2 mL of acetone at room temperature; the resulting solution (5 mg mL⁻¹) was added drop wise to 10 mL of distilled water under vigorous stirring for ca. 15 min using a glass pipette. The solution was stirred vigorously for 24 h at room temperature, until the acetone was completely evaporated as determined by ¹H NMR analysis, where no peak at δ 2.22 corresponding to acetone was observed.

4.2 Nanoprecipitation (Fast Addition)

Polydendrons were dissolved in THF for a minimum of 6 hours at various concentrations. Once fully dissolved polymer in THF (1 ml, 5 mg/ml) was added quickly to a vial of water (5 ml) stirring at 30° C. The solvent was allowed to evaporate overnight in a fume cupboard to give a final concentration of 1 mg/ml polymer in water. By adjusting the starting concentration and the volume of water used, the size of the corresponding nanoparticles can be controlled to an extent. The nanoparticles formed were analysed by dynamic light scattering (DLS) and fluorimetry.

TABLE 3
DLS data tor 100% Dendron initiated polydendrons

| | Polymer | EGDMA | Size | |
|---|---|---|---|---|
| Initiator | core | (mol %) | (d · nm) | PDI |
| G1 DBOP | pHPMA20 | 0.8 | 61.72 | 0.117 |
| G1 DBOP | pHPMA50 | 0.8 | 63.9 | 0.130 |
| G1 DBOP | pHPMA100 | 0.8 | 69.89 | 0.070 |
| G2 DBOP | pHPMA20 | 0.8 | 81.33 | 0.076 |
| G2 DBOP | pHPMA50 | 0.8 | 80.78 | 0.083 |
| G2 DBOP | pHPMA100 | 0.8 | 80.56 | 0.119 |
| G1-A tamine | pHPMA50 | 0.7 | 70.6 | 0.366 |
| G1-A tamine | ptBuMA50 | 0.95 | 45.98 | 0.217 |
| G1-A tamine | pDEAEMA50 | 0.9 | 136.2 | 0.148 |
| G1-A tamine | pOEGMA50 | 0.95 | 44.98 | 0.519 |
| G0-D tamine | pDEAEMA50 | 0.9 | | |
| G1-D tamine | pDEAEMA50 | 0.9 | | |
| G2-D tamine | pDEAEMA50 | 0.9 | 115.9 | 0.158 |
| G2-D tamine | pDEAEMA50-block- | 0.9 | 162.9 | 0.082 |
| | tBuMA-st-EGDMA | | | |
| Xant G1 - post | | | | |
| modified with; | | | | |
| benzyl | pHPMA50 | 0.85 | 141.1 | 0.238 |
| n-morpholino | pHPMA50 | 0.85 | 159.3 | 0.166 |
| PEG480 | pHPMA50 | 0.85 | 106.9 | 0.257 |
| PEG5000 | pHPMA50 | 0.85 | 156.8 | 0.427 |

TABLE 4
DLS data for mixed initiator polydendrons

| | | Polymer | EGDMA | | |
|---|---|---|---|---|---|
| Initiator 1 | Initiator 2 | core | (mol %) | Size (d · nm) | PDI |
| G1 tBOC | bifunctional | pHPMA50 | 0.95 | 73.78 | 0.109 |
| G2 tBOC | bifunctional | pHPMA50 | 0.8 | 27.33 | 0.116 |
| G2 DBOP | 750 PEG | | | | |
| 100 | 0 | pHPMA50 | 0.8 | 80.78 | 0.083 |
| 90 | 10 | pHPMA50 | 0.8 | 115.6 | 0.069 |
| 75 | 25 | pHPMA50 | 0.8 | 109.8 | 0.073 |
| 50 | 50 | pHPMA50 | 0.8 | 114.6 | 0.067 |
| 25 | 75 | pHPMA50 | 0.8 | 92.57 | 0.078 |
| 10 | 90 | pHPMA50 | 0.8 | 94.26 | 0.091 |
| 0 | 100 | pHPMA50 | 0.8 | 87.8 | 0.076 |
| 0 | 100 | pHPMA50 | 0.95 | 89.53 | 0.083 |
| G2 DBOP | 2K PEG | | | | |
| 100 | 0 | pHPMA50 | 0.8 | 62.15 | 0.391 |
| 90 | 10 | pHPMA50 | 0.8 | 144.4 | 0.036 |
| 75 | 25 | pHPMA50 | 0.8 | 214.6 | 0.085 |
| 50 | 50 | pHPMA50 | 0.85 | 105.5 | 0.058 |
| 25 | 75 | pHPMA50 | 0.95 | 52.17 | 0.277 |
| 10 | 90 | pHPMA50 | 0.95 | 37.81 | 0.207 |
| 0 | 100 | pHPMA50 | 0.95 | 36.18 | 0.24 |
| 50 | 50 | pHPMA20 | 0.85 | 54.9 | 0.296 |
| 50 | 50 | pHPMA100 | 0.8 | 232.2 | 0.133 |

### 5. Encapsulation of Fluorescent Molecules

5.1 Nile Red Encapsulation—HR Method

In a typical procedure, 10 mg of sample and 0.1 mg Nile Red was dissolved completely in 2 mL of acetone at room temperature; the resulting solution (5.05 mg mL⁻¹) was added drop wise to 10 mL of distilled water under vigorous stirring for ca. 15 min using a glass pipette. The solution was stirred vigorously for 24 h at room temperature, until the acetone was completely evaporated as determined by ¹H NMR analysis, where no peak at δ 2.22 corresponding to acetone was observed.

5.2 Fluoresceinamine Encapsulation—HR Method

In a typical procedure, 10 mg of sample and 1 mg of fluoresceinamine was dissolved completely in 2 mL of acetone at room temperature; the resulting solution (5.5 mg mL⁻¹) was added drop wise to 10 mL of distilled water under vigorous stirring for ca. 15 min using a glass pipette. The solution was stirred vigorously for 24 h at room temperature, until the acetone was completely evaporated as determined by ¹H NMR analysis, where no peak at δ 2.22 corresponding to acetone was observed.

5.3 Encapsulation of Nile Red or Pyrene Using Mixed Initiator Polydendrons

Stock solutions of nile red in THF at 0.2 mg/ml and pyrene in THF at 0.5 mg/ml were made. In a typical experiment the desired amount of nile red or pyrene was added to a vial using a pipette (e.g. for a stock solution at 0.2 mg/ml, 100 μl would be used if 0.02 mg was required). The vial was left in the fume cupboard for 20 min to allow evaporation of the THF. A pre-dissolved sample of polymer in THF (1 ml, 5 mg/ml) was added to the vial. The vial was shaken gently to allow dissolution of the fluorescent molecule in the THF containing polymer. Once the desired amount of polymer and fluorescent molecule was dissolved in the 1 ml of THF, this was added quickly to a vial of water (5 ml) stirring at 30° C. The solvent was allowed to evaporate in a fume cupboard overnight, giving a final concentration of 1 mg/ml polymer in water. The nanoparticles formed were analysed by dynamic light scattering (DLS) and fluorimetry.

Table 5 shows data for polymer nanoparticles with a final concentration of 1 mg/ml polymer with 0.1 w/w % nile red or pyrene encapsulated (1 μg/ml)

TABLE 5
Fluorimetry of nanoparticles with nile red and pyrene encapsulated

| | | | | Nile red | |
|---|---|---|---|---|---|
| | | | | encapsu- | |
| | | | | lation | Pyrene |
| | | | | (max | encapsu- |
| Initiator 1 | Initiator 2 | Polymer | EGDMA | intensity at | lation |
| G2 DBOP | 750 PEG | core | (mol %) | 630 nm) | I1/I3 ratio |
| 100 | 0 | pHPMA50 | 0.8 | 702.1693 | 1.42 |
| 90 | 10 | pHPMA50 | 0.8 | 625.9234 | 1.4458 |
| 75 | 25 | pHPMA50 | 0.8 | 574.7425 | 1.4666 |
| 50 | 50 | pHPMA50 | 0.8 | 548.357 | 1.4685 |
| 25 | 75 | pHPMA50 | 0.8 | 243.2502 | 1.479 |
| 10 | 90 | pHPMA50 | 0.8 | 404.1123 | 1.5208 |
| 0 | 100 | pHPMA50 | 0.8 | 285.757 | 1.5315 |
| 0 | 100 | pHPMA50 | 0.95 | 226.2446 | — |

### 6. Pharmacology

1. Materials & Methods

1. Materials

Dulbecco's Modified Eagles Medium (DMEM), Hanks buffered saline solution (HBSS), Trypsin-EDTA, bovine serum albumin (BSA), Nile red, 3-(4,5-Dimethylthiazol-2-yl)-2,5-diphenyltetrazolium bromide (MTT reagent), acetonitrile (ACN) and all general laboratory reagents were purchased from Sigma (Poole, UK). Foetal bovine serum (FBS) was purchased from Gibco (Paisley, UK). The CellTiter-Glo® Luminescent Cell Viability Assay kit was from Promega (UK). The 24-well HTS transwell plates were obtained from Corning (New York, USA). The 96-well black walled, flat bottomed plates were from Sterilin (Newport, UK).

1.1 Routine Cell Culture/Cell Maintenance

Caco-2 cells were purchased from American Type Culture Collection (ATCC, USA) and maintained in Dulbecco's Modified Eagles Medium (DMEM) supplemented with 15% filtered sterile foetal bovine serum. Cells were incubated at 37° C. and 5% CO₂ and were routinely sub-cultured every 4 days when 90% confluent. Cell count and viability was determined using a Countess automated cell counter (Invitrogen).

1.2 Cytotoxicity

Caco-2 cells were seeded at a density of 1.0×10⁴ cells/100 μl in DMEM supplemented with 15% FBS into each well of a 96 well plate (Nunclon, Denmark) and incubated at 37° C. and 5% CO₂. Cells from 4 separate flasks of biological replicates of each cell type were used (N1-4) to improve statistical power. Media was then aspirated from column 1 and replaced with media containing each polydendron or aqueous Nile Red solution at an equivalent 1 μM Nile Red concentration then diluted 1:1 in media across the plate up to column 11. Column 12 served as a negative control and consisted of media and untreated cells. Following polydendron addition, the plates were incubated for 24 hours or 120 hours at 37° C., 5% CO₂ prior to assessment of cytotoxicity.

1.3 MTT Assay

Following incubation of treated plates for 24 h or 120 h, 20 μl of 5 mg ml⁻¹ MTT reagent was added to each well and incubated for 2 hours. Subsequently, 100 μL MTT lysis buffer (50% N—N-Dimethylformamide in water containing 20% SDS, 2.5% glacial acetic acid and 2.5% hydrochloric acid, pH 4.7) was added to each well to lyse overnight at 37° C., 5% CO₂. Following incubation the absorbance of each well was read using a Tecan Genosis plate reader at 560 nm (Tecan Magellan, Austria).

1.4 ATP Assay

Following incubation of treated plates for 24 h or 120 h, cells were equilibrated to room temperature for approximately 30 minutes. All but 20 μl of media was removed from each well and 20 μl CellTiter-Glo® (Promega, UK) reagent was added. All reagents were made fresh and in accordance with the manufacturer's instructions. Plates were put on an orbital shaker for 10 minutes to mix contents and allow for stabilisation of luminescence signal. Luminescence was then measured using a Tecan Genios plate reader (Tecan Magellan, Austria).

2. Transcellular Permeability of Nile Red Across Caco-2 Monolayers

2.1 Setting Up and Treating Transwell Plates

Transwells were seeded with 3.5×10⁴ cells per well and propagated to a monolayer over a 21 day period, during which media in the apical and basolateral wells was changed every other day. Trans-epithelial electrical resistance (TEER) values were monitored until they were >1300Ω. 1 μM of Nile Red polydendron or 1 μM aqueous Nile Red was added to the apical chamber of 4 wells and the basolateral chamber of 4 wells to quantify transport in both Apical to Basolateral (A>B) and Basolateral to Apical (B>A) direction and sampled on an hourly basis over a 4 h time period. Apparent permeability coefficient was then determined by the amount of compound transported over time using the equation:

Papp=(dQ/dt)(1/AC₀)

where (dQ/dt) is the amount per time (nmol·sec⁻¹), A is the surface area of the filter and C₀ is the starting concentration of the donor chamber (1 μM).

2.3 Extraction and Quantification of Nile Red

100 μl of each collected sample was mixed with 900 μl acetone, vortexed, sonicated for 6 minutes and centrifuged at 13300 rpm for 3 minutes. The supernatant was completely dried in a vacuum centrifuge at 30° C. until the dry solid sample was left. This was reconstituted in 150 μl acetonitrile, transferred to a 96-well black walled, flat bottomed plate and measured for fluorescence intensity excitation wavelength 480 nm, emission wavelength 560 nm using a Tecan Genios plate reader (Tecan Magellan, Austria).

3. Results

3.1 Cytotoxicity—MTT Assays

Following 24 hour incubation of Caco-2 cells with each polydendron, analysis of cytotoxicity by MTT assay (FIG. 6) showed that aqueous Nile Red and each polydendron did not affect metabolic turnover of Caco-2 cells compared to untreated cells at the range of concentrations investigated. It can be inferred that metabolic turnover correlates to cell viability in which case each material was not cytotoxic.

FIG. 6: MTT assay of Caco-2 cells following 24 hour incubation with aqueous Nile Red and each polydendron. A=aqueous Nile Red, EC₃₀ 1.160. B=0:100, EC₅₀ 2.509. C=10:90, EC₅₀ 1.410. D=25:75, EC₅₀ 1.567. E=50:50, EC₅₀ 1.083. F=75:25, EC₅₀ 1.565, G=90:10, EC₅₀ 1.607. H=100:0, EC₅₀ 2.678.

Following 120 hour incubation of Caco-2 cells with each polydendron, analysis of cytotoxicity by MTT assay (FIG. 7) showed that aqueous NR and each polydendron at the range of concentrations investigated did not affect the viability of Caco-2 cells.

FIG. 7: MTT assay of Caco-2 cells following 120 hour incubation with aqueous Nile Red and each polydendron. A=aqueous Nile Red, EC₅₀ No EC₅₀. B=0:100, EC₅₀ 1.528. C=10:90, EC₅₀ No EC₅₀. D=25:75, EC₅₀ 6.166. E=50:50, EC₅₀ 0.7856. F=75:25, EC₅₀ No EC₅₀, G=90:10, EC₅₀ 0.2176. H=100:0, EC₅₀ No EC₅₀.

3.3 ATP Assay

Following 24 hour incubation of Caco-2 cells with each polydendron, analysis of cytotoxicity by ATP assay using a CellTiter-Glo® kit (Promega, UK) (FIG. 8) indicated that ATP presence was not affected in cells treated with aqueous Nile Red solution and polydendron formulated Nile Red at the range of concentrations investigated compared to untreated cells. It can be inferred that the presence of ATP correlates to cell viability in which case each material was not cytotoxic.

FIG. 8: ATP assay of Caco-2 cells following 24 hour incubation with aqueous Nile Red and each polydendron. A=aqueous Nile Red, EC₅₀ 1.946. B=0:100, EC₅₀ 2.855. C=10:90, EC₅₀ No EC₅₀. D=25:75, EC₅₀ No EC₅₀. E=50:50, EC₅₀ No EC₅₀. F:=75:25, EC₅₀ No EC₅₀, G=90:10, EC₅₀ 2.848. H=100:0, EC₅₀ 0.1961.

Following 120 hour incubation of Caco-2 cells with each polydendron, analysis of cytotoxicity by ATP assay using a CellTiter-Glo® kit (Promega, UK) (FIG. 9) indicated viability was not affected in cells treated with aqueous Nile Red solution and each polydendron material at the range of concentrations investigated compared to untreated cells.

FIG. 9: ATP assay of Caco-2 cells following 120 hour incubation with aqueous Nile Red and each polydendron. A=aqueous Nile Red, EC₅₀ No EC₅₀. B=0:100, EC₅₀ No EC₅₀. C=10:90, EC₅₀ 3.168. D=25:75, EC₅₀ 2.565. E=50:50, EC₅₀ No EC₅₀. F=75:25, EC₅₀ 3.032, G=90:10, EC₅₀ No EC₅₀. H=100:0, EC₅₀ No EC₅₀.

4. Transcellular Permeability of Selected Nile Red Polydendron Materials Across Caco-2 Cell Monolayers.

Transcellular permeability of Nile Red through Caco-2 cell monolayers (to model the intestinal epithelium) was significantly higher in the apical to basolateral (A>B, gut to blood) direction for the polydendron preparation 10G2:90PEG compared to an aqueous solution of Nile Red (FIGS. 10 A&B). All the polydendron materials produced a greater apical to basolateral (A>B, gut to blood), basolateral to apical (B>A, blood to gut) ratio than an aqueous preparation of Nile Red following 1 hour incubation (Table 1, FIG. 10 C). A statistically significant correlation (P=<0.05) between the ratio of dendron and PEG used in the polydendron formulation and the ratio of apical to basolateral (A>B, gut to blood), basolateral to apical (B>A, blood to gut) movement of Nile Red across the Caco-2 monolayer was observed (FIG. 10 C).

FIG. 10. (A&B) Transcellular permeability across Caco2 cell monolayers of polydendron formulated Nile Red relative to an aqueous solution of Nile Red. Data are given as the mean of experiments conducted in biological triplicate. (C) Correlation between polydendron formulation and the ratio of Nile Red transported (A>B/B>A) across Caco2 cell monolayers (r² 0.784). Data were normally distributed, statistical analysis was conducted using a Pearson correlation (P=<0.05) a two-tailed P value was used to reduce the chance of a type I error.

TABLE 1
Apparent permeability (Papp) of Nile Red polydendrons and aqueous
Nile Red across Caco2 cell monolayers following 1 hour incubation.
Data are given as the mean of experiments conducted in
biological triplicate.

| Papp (cm s⁻¹) | | | |
|---|---|---|---|
| Polydendron | | | |
| Formulation | | | A > B/B > A |
| (G2:PEG ratio) | Apical > Basolateral | Basolateral > Apical | ratio |
| 1.00 | 1.763 × 10⁻⁵ | 1.538 × 10⁻⁶ | 11.4605 |
| 0.75 | 2.613 × 10⁻⁵ | 2.056 × 10⁻⁶ | 12.7123 |
| 0.50 | 5.271 × 10⁻⁵ | 5.555 × 10⁻⁶ | 9.4872 |
| 0.25 | 4.135 × 10⁻⁵ | 4.684 × 10⁻⁶ | 8.8279 |
| 0.10 | 4.042 × 10⁻⁴ | 4.580 × 10⁻⁵ | 8.8255 |
| 0.00 | 2.060 × 10⁻⁵ | 3.188 × 10⁻⁶ | 6.4626 |
| Aqueous Nile | 2.371 × 10⁻⁵ | 6.384 × 10⁻⁶ | 3.7140 |
| Red | | | |

### 7. Preparation of Acid Cleavable Brancher (BDME)

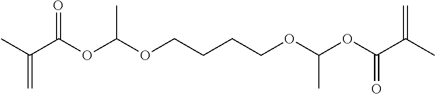 [SMILES: C=C(C)C(=O)OC(C)OCCCCOC(C)OC(=O)C(=C)C]

BDVE (5.6 ml, 35.21 mmol) was added to a two-necked 250 ml round bottomed flask equipped with a condenser, a magnetic stirrer and a positive flow of nitrogen. A small amount of radical inhibitor 4-tert-butylcatechol (end of a spatula) was added and the mixture deoxygenated using a nitrogen purge for 15 minutes. Once dissolved, the temperature was raised to 70° C. MAA (14.9 ml, 175.8 mmol) was added dropwise over 10 minutes through a septa. The reaction was allowed to proceed at 70° C. for a further 6 hours with stirring. After this time, the reaction was stopped by cooling and exposing to the air. The crude product was dissolved in chloroform (100 ml) and washed with basic H₂O (pH2, 3×100 ml). The combined washings were collected and dried over NaSO₄ and the solvent removed by rotary evaporation.

(Found: C, 61.45; H, 8.28%. C₁₆H₂₆O₆ requires C, 61.15; H, 8.28%); ¹H NMR (400 MHz; CDCl₃; Me₄Si) δ 1.44 (61H, d, CH₃CH), 1.65 (4H, m, CH₂CH₂CH₂), 1.95 (6H, s, CH₃C═CH2), 3.50-3.69 (4H, m, OCH₂CH₂), 5.60 and 6.15 (4H, 2s, CH₂═CCH₃) and 5.95-5.99 (2H, q, CHCH₃). ¹³C NMR (400 MHz; CDCl₃; Me₄Si) δ 18.27 (s), 20.83 (s), 26.29 (s) 68.85 (s), 96.93 (s), 125.90 (s), 136.37 (s) and 167.01 (s). m/z (EI) 314.2 (M⁺—C₁₆H₂₆O₆ requires 314).

### 8. DEAEMA Polydendron Synthesis

8.1 Polymerisation of G1-A Dendron Initiated DEAEMA₅₀

In a typical synthesis, targeting a number average degree of polymerisation (DPₙ)=50 monomer units (poly(DEAEMA)₅₀; n_{DEAEMA}/n_{Initiator}: 50), bpy (134.9 mg, 0.8637 mmol, 2 eq.), DEAEMA (4 g, 21.59 mmol, 50 eq.) and isopropanol (IPA) (56% v/v based on DEAEMA) were placed into a 25 mL round-bottomed flask. The solution was stirred and deoxygenated using a nitrogen (N₂) purge for 15 minutes. Cu(ᵢ)Cl (42.8 mg, 0.4318 mmol, 1 eq.) was added to the flask and left to purge for a further 5 minutes. G1-A dendron initiator (0.2576 g, 0.4318 mmol, 1 eq.) was added to the flask under a positive flow of N₂, and the solution was left to polymerise at 40° C. Reactions were terminated when >99% conversion was reached, as judged by ¹H NMR, by exposure to oxygen and addition of acetone. The catalyst residues were removed by passing the mixture over a basic alumina column. Acetone was removed under vacuum to concentrate the sample before precipitation into cold petroleum ether (40° C.-60° C.) and drying in the vacuum oven overnight.

| | Mw/Da | Mn/Da | PDI | Z-Ave/nm | PdI |
|---|---|---|---|---|---|
| DEAEMA₅₀ | 23,579 | 19,783 | 1.2 | 119.9 | 0.113 |

8.2 Polymerisation of G1-A dendron initiated DEAEMA₅₀-EGDMA_{0.9}

In a typical synthesis, targeting a number average degree of polymerisation (DPₙ)=50 monomer units (poly(DEAEMA)₅₀; n_{DEAEMA}/n_{Initiator}: 50), bpy (134.9 mg, 0.8637 mmol, 2 eq.), DEAEMA (4 g, 21.59 mmol, 50 eq.), EGDMA (77.0 mg, 0.3886 mmol, 0.9 eq) and IPA³⁷ (38.9% v/v based on DEAEMA) were placed into a 25 mL round-bottomed flask. The solution was stirred and deoxygenated using a N₂ purge for 15 minutes. Cu(ᵢ)Cl (42.8 mg, 0.4318 mmol, 1 eq.) was added to the flask and left to purge for a further 5 minutes. G1-A dendron initiator (0.2576 g, 0.4318 mmol, 1 eq.) was added to the flask under a positive flow of N₂, and the solution was left to polymerise at 40° C. Reactions were terminated when >99% conversion was reached, as judged by ¹H NMR, by exposure to oxygen and addition of acetone. The catalyst residues were removed by passing the mixture over a basic alumina column. Acetone was removed under vacuum to concentrate the sample before precipitation into cold petroleum ether (40° C.-60° C.). The polymerisation conditions and procedure is identical to those described for linear polymers above and drying in the vacuum oven overnight.

| | Mw/Da | Mn/Da | PDI | -Ave/nm | PdI |
|---|---|---|---|---|---|
| DEAEMA₅₀- | 244,622 | 201,497 | 1.2 | 136.2 | 0.148 |
| EGDMA₅₀ | | | | | |

8.3 polymerisation of G1-A dendron initiated pDEAEMAₓ-BDME_{2.0}

in a Typical Synthesis, Targeting a Degree of Polymerisation (DP)=50 Monomer Units (pDEAEMA₅₀), bipy (160.8 mg, 0.8637 mmol), BDME (271.2 mg, 0.864 mmol) and DEAEMA (4.08 g, 21.59 mmol) in IPA (4 ml, 56 v/v % based on DEAEMA) were added to a 25 ml round bottomed flask. A brancher to initiator ratio of 1:2 was applied for the branching polymerisation The solution was stirred and deoxygenated for 15 minutes using a nitrogen purge. Cu(I)Cl (42.8 mg, 0.4318 mmol) was quickly added to the flask under a positive flow of nitrogen and the mixture deoxygenated for a further 5 minutes. A dark brown solution resulted. G1-A (0.2576 g, 0.4318 mmol)—was added through the septa and the solution was left to polymerise at 40° C. for 24 hours. After this time, the reaction was terminated by exposure to oxygen and vigorously stirring in acetone (100 ml). The mixture turned green after 20 minutes. The catalyst residues were removed from the reaction mixture by passing the mixture over a basic alumina column. The solvent was removed under reduced pressure and the crude polymer redissolved in a minimum volume of acetone (10 ml) before precipitation into cold petroleum ether. The resulting polymer was dried overnight at 40° C. in a vacuum oven. For determining monomer conversion (%), the reaction was sampled and diluted into CDCl₃ for 1H NMR analysis.

| | Mw/Da | Mn/Da | PDI | Z-Ave/nm | PdI |
|---|---|---|---|---|---|
| DEAEMA₅₀- | 353,759 | 62,918 | 5.62 | 238.2 | 0.115 |
| EGDMA_{2.0} | | | | | |
| DEAEMA₅₀- | 702,665 | 426,086 | 1.65 | 287.0 | 0.174 |
| BDME_{2.0} | | | | | |

8.4 Polymerisation of G0-D Dendron Initiated DEAEMA₅₀

In a typical synthesis, targeting a number average degree of polymerisation (DPₙ)=50 monomer units (poly(DEAEMA)₅₀; n_{DEAEMA}/n_{Initiator}: 50), bpy (134.9 mg, 0.8637 mmol, 2 eq.), DEAEMA (4 g, 21.59 mmol, 50 eq.) and isopropanol (IPA) (56% v/v based on DEAEMA) were placed into a 25 mL round-bottomed flask. The solution was stirred and deoxygenated using a nitrogen (N₂) purge for 15 minutes. Cu(ᵢ)CI (42.8 mg, 0.4318 mmol, 1 eq.) was added to the flask and left to purge for a further 5 minutes. G0-D dendron initiator (0.1089 g, 0.4318 mmol, 1 eq.) was added to the flask under a positive flow of N₂, and the solution was left to polymerise at 40° C. Reactions were terminated when >99% conversion was reached, as judged by ¹H NMR, by exposure to oxygen and addition of acetone. The catalyst residues were removed by passing the mixture over a basic alumina column. Acetone was removed under vacuum to concentrate the sample before precipitation into cold petroleum ether (40° C.-60° C.) and drying in the vacuum oven overnight.

| | Mw/Da | Mn/Da | PDI | Z-Ave/nm | PdI |
|---|---|---|---|---|---|
| DEAEMA₅₀ | 26,295 | 14,271 | 1.8 | 62.2 | 0.177 |

8.5 Polymerisation of G0-D dendron initiated DEAEMA₅₀-EGMA_{0.9} in a Typical Synthesis, Targeting a Number Average Degree of Polymerisation (DPₙ)⁼50 monomer units (poly(DEAEMA)₅₀; n_{DEAEMA}/n_{Initiator}: 50), bpy (134.9 mg, 0.8637 mmol, 2 eq.), DEAEMA (4 g, 21.59 mmol, 50 eq.), EGDMA (77.0 mg, 0.3886 mmol, 0.9 eq) and IPA³⁷ (38.9% v/v based on DEAEMA) were placed into a 25 mL round-bottomed flask. The solution was stirred and deoxygenated using a N₂ purge for 15 minutes. Cu(ᵢ)Cl (42.8 mg, 0.4318 mmol, 1 eq.) was added to the flask and left to purge for a further 5 minutes. G0-D dendron initiator (0.1089 g, 0.4318 mmol, 1 eq.) was added to the flask under a positive flow of N₂, and the solution was left to polymerise at 40° C. Reactions were terminated when >99% conversion was reached, as judged by ¹H NMR, by exposure to oxygen and addition of acetone. The catalyst residues were removed by passing the mixture over a basic alumina column. Acetone was removed under vacuum to concentrate the sample before precipitation into cold petroleum ether (40° C.-60° C.) and drying in the vacuum oven overnight. The polymerisation conditions and procedure is identical to those described for linear polymers above.

| | Mw/Da | Mn/Da | PDI | Z-Ave/nm | PdI |
|---|---|---|---|---|---|
| DEAEMA₅₀- | 184,394 | 41,431 | 4.45 | 127.8 | 0.089 |
| EGDMA_{0.9} | | | | | |

8.6 Polymerisation of G1-D Dendron Initiated DEAEMA₅₀

In a typical synthesis, targeting a number average degree of polymerisation (DPₙ)=50 monomer units (poly(DEAEMA)₅₀; n_{DEAEMA}/n_{Initiator}: 50), bpy (134.9 mg, 0.8637 mmol, 2 eq.), DEAEMA (4 g, 21.59 mmol, 50 eq.) and isopropanol (IPA) (56% v/v based on DEAEMA) were placed into a 25 mL round-bottomed flask. The solution was stirred and deoxygenated using a nitrogen (N₂) purge for 15 minutes. Cu(ᵢ)CI (42.8 mg, 0.4318 mmol, 1 eq.) was added to the flask and left to purge for a further 5 minutes. G1-D dendron initiator (0.2204 g, 0.4318 mmol, 1 eq.) was added to the flask under a positive flow of N₂, and the solution was left to polymerise at 40° C. Reactions were terminated when >99% conversion was reached, as judged by ¹H NMR, by exposure to oxygen and addition of acetone. The catalyst residues were removed by passing the mixture over a basic alumina column. Acetone was removed under vacuum to concentrate the sample before precipitation into cold petroleum ether (40° C.-60° C.) and drying in the vacuum oven overnight.

| | Mw/Da | Mn/Da | PDI | Z-Ave/nm | PdI |
|---|---|---|---|---|---|
| DEAEMA₅₀ | 59,416 | 31,542 | 1.9 | 134.7 | 0.103 |

8.7 Polymerisation of G1-D Dendron Initiated DEAEMA₅₀-EGDMA_{0.9}

In a typical synthesis, targeting a number average degree of polymerisation (DPₙ)=50 monomer units (poly(DEAEMA)₅₀; n_{DEAEMA}/n_{Initiator}: 50), bpy (134.9 mg, 0.8637 mmol, 2 eq.), DEAEMA (4 g, 21.59 mmol, 50 eq.), EGDMA (77.0 rag, 0.3886 mmol, 0.9 eq) and IPA³⁷ (38.9% v/v based on DEAEMA) were placed into a 25 mL round-bottomed flask. The solution was stirred and deoxygenated using a N₂ purge for 15 minutes. Cu(ᵢ)Cl (42.8 mg, 0.4318 mmol, 1 eq.) was added to the flask and left to purge for a further 5 minutes. G1-D dendron initiator (0.2204 g, 0.4318 mmol, 1 eq.) was added to the flask under a positive flow of N₂, and the solution was left to polymerise at 40° C. Reactions were terminated when >99% conversion was reached, as judged by ¹H NMR, by exposure to oxygen and addition of acetone. The catalyst residues were removed by passing the mixture over a basic alumina column. Acetone was removed under vacuum to concentrate the sample before precipitation into cold petroleum ether (40° C.-60° C.) and drying in the vacuum oven overnight. The polymerisation conditions and procedure is identical to those described for linear polymers above.

| | Mw/Da | Mn/Da | PDI |
|---|---|---|---|
| DEAEMA₅₀- | 173024 | 61944 | 2.79 |
| EGDMA_{0.9} | | | |

8.8 Polymerisation of G2-D Dendron Initiated DEAEMA₅₀

In a typical synthesis, targeting a number average degree of polymerisation (DPₙ)=50 monomer units (poly(DEAEMA)₅₀; n_{DEAEMA}/n_{Initiator}: 50), bpy (134.9 mg, 0.8637 mmol, 2 eq.), DEAEMA (4 g, 21.59 mmol, 50 eq.) and isopropanol (IPA) (56% v/v based on DEAEMA) were placed into a 25 mL round-bottomed flask. The solution was stirred and deoxygenated using a nitrogen (N₂) purge for 15 minutes. Cu(ᵢ)Cl (42.8 mg, 0.4318 mmol, 1 eq.) was added to the flask and left to purge for a further 5 minutes. G2-D dendron initiator (0.3934 g, 0.4318 mmol, 1 eq.) was added to the flask under a positive flow of N₂, and the solution was left to polymerise at 40° C. Reactions were terminated when >99% conversion was reached, as judged by ¹H NMR, by exposure to oxygen and addition of acetone. The catalyst residues were removed by passing the mixture over a basic alumina column. Acetone was removed under vacuum to concentrate the sample before precipitation into cold petroleum ether (40° C.-60° C.) and drying in the vacuum oven overnight.

| | Mw/Da | Mn/Da | PDI | Z-Ave/nm | PdI |
|---|---|---|---|---|---|
| DEAEMA₅₀ | 34,386 | 21,553 | 1.6 | 110.9 | 0.123 |

8.9 Polymerisation of G2-D Dendron Initiated DEAEMA₅₀-EGDMA_{0.9}

In a typical synthesis, targeting a number average degree of polymerisation (DPₙ)=50 monomer units (poly(DEAEMA)₅₀; n_{DEAEMA}/n_{Initiator}: 50), bpy (134.9 mg, 0.8637 mmol, 2 eq.), DEAEMA (4 g, 21.59 mmol, 50 eq.), EGDMA (77.0 mg, 0.3886 mmol, 0.9 eq) and IPA³⁷ (38.9% v/v based on DEAEMA) were placed into a 25 mL round-bottomed flask. The solution was stirred and deoxygenated using a N₂ purge for 15 minutes. Cu(ᵢ)Cl (42.8 mg, 0.4318 mmol, 1 eq.) was added to the flask and left to purge for a further 5 minutes. G2-D dendron initiator (0.3934 g, 0.4318 mmol, 1 eq.) was added to the flask under a positive flow of N₂, and the solution was left to polymerise at 40° C. Reactions were terminated when >99% conversion was reached, as judged by ¹H NMR, by exposure to oxygen and addition of acetone. The catalyst residues were removed by passing the mixture over a basic alumina column. Acetone was removed under vacuum to concentrate the sample before precipitation into cold petroleum ether (40° C.-60° C.) and drying in the vacuum oven overnight. The polymerisation conditions and procedure is identical to those described for linear polymers above.

| | Mw/Da | Mn/Da | PDI | Z-Ave/nm | PdI |
|---|---|---|---|---|---|
| DEAEMA₅₀- | 302,557 | 125,652 | 2.4 | 115.9 | 0.158 |
| EGDMA_{0.9} | | | | | |

### 9. Hydrolysis of Branched pDEAEMAx

Hydrolysis of branched pDEAEMA₅₀ was carried out in acetone at room temperature in the presence of a small amount of aqueous HCl, with magnetic stirring. Solutions of each branched polymer in acetone were prepared (40 mg/ml, 9 ml). HCl (6M, 300 μl) was added dropwise to each of the polymer solutions. The solutions were stirred vigorously at room temperature for 20 minutes, resulting in a cloudy solution with solid precipitate. Distilled water (9 ml) was added to each of the acidic polymer solutions. The solutions were allowed to stir overnight in a sealed vial. Each of the hydrolysed polymer solutions were frozen in liquid nitrogen and lyophilised for 72 hours, then dissolved in a THF/2 v/v % TEA eluent system and analysed by GPC.

| | Z-Ave/nm | PdI |
|---|---|---|
| DEAEMA₅₀- | 73.4 | 0.330 |
| EGDMA_{2.0} | | |
| DEAEMA₅₀- | 108.9 | 0.202 |
| BDME_{2.0} | | |

### 10. Co-Polydendron Synthesis

10.1 Co-polymerisation of G2-D dendron initiated DEAEMA₅₀-tBuMA₆₅

In a typical synthesis, targeting a number average degree of polymerisation (DPₙ)=50 monomer units (poly(DEAEMA)₅₀; n_{DEAEMA}/n_{Initiator}: 50), bpy (134.9 mg, 0.8637 mmol, 2 eq.), DEAEMA (4 g, 21.59 mmol, 50 eq.) and isopropanol (IPA) (37.7% v/v based on DEAEMA) were placed into a 50 mL round-bottomed flask. The solution was stirred and deoxygenated using a nitrogen (N₂) purge for 15 minutes. Cu(ᵢ)Cl (42.8 mg, 0.4318 mmol, 1 eq.) was added to the flask and left to purge for a further 5 minutes. G2-D dendron initiator (0.3934 g, 0.4318 mmol, 1 eq.) was added to the flask under a positive flow of N₂, and the solution was left to polymerise at 40° C. In another 25 mL round-bottomed flask, bpy (134.9 mg, 0.8637 mmol), tBuMA (4.0 g, 21.59 mmol) and aqueous isopropanol (23.8% v/v based on tBuMA) were added. The solution was stirred and deoxygenated using a nitrogen (N₂) purge for 15 minutes. Cu(ᵢ)Cl (42.8 mg, 0.4318 mmol, 1 eq.) was added to the flask and left to purge for a further 5 minutes. After the conversion of DEAEMA reached around 85%, the mixture from the second flask was added into the first flask rapidly using a syringe and taking care not to admit any air into the vessel. A sample was taken immediately after the addition of the tBuMA monomer solution for ¹H NMR analysis. The block copolymerization reaction was carried out at ambient temperature and samples were taken periodically from the reaction mixture for ¹H NMR analysis. Reactions were terminated when >99% conversion was reached, as judged by ¹H NMR, by exposure to oxygen and addition of acetone. The catalyst residues were removed by passing the mixture over a basic alumina column. Acetone was removed under vacuum to concentrate the sample before precipitation into cold petroleum ether (40° C.-60° C.) and drying in the vacuum oven overnight.

| | Mw/Da | Mn/Da | PDI | Z-Ave/nm | PdI |
|---|---|---|---|---|---|
| DEAEMA₅₀ | 92,617 | 80,687 | 1.1 | 38.42 | 0.244 |
| tBuMA₆₅ | | | | | |

10.2 Co-Polymerisation of G2-D Dendron Initiated DEAEMA₅₀-tBuMA₆₅-EGDMA_{0.9}

In a typical synthesis, targeting a number average degree of polymerisation (DPₙ)=50 monomer units (poly(DEAEMA)₅₀; n_{DEAEMA}/n_{Initiator}: 50), bpy (134.9 mg, 0.8637 mmol, 2 eq.), DEAEMA (4 g, 21.59 mmol, 50 eq.) and isopropanol (IPA) (37.7% v/v based on DEAEMA) were placed into a 50 mL round-bottomed flask. The solution was stirred and deoxygenated using a nitrogen (N₂) purge for 15 minutes. Cu(ᵢ)Cl (42.8 mg, 0.4318 mmol, 1 eq.) was added to the flask and left to purge for a further 5 minutes. G2-D dendron initiator (0.3934 g, 0.4318 mmol, 1 eq.) was added to the flask under a positive flow of N₂, and the solution was left to polymerise at 40° C. In another 25 mL round-bottomed flask, bpy (134.9 mg, 0.8637 mmol), tBuMA (4.0 g, 28.1 mmol, 65 eq.), EGDMA (77.0 mg, 0.3886 mmol, 0.9 eq) and aqueous isopropanol (23.8% v/v based on tBuMA) were added. The solution was stirred and deoxygenated using a nitrogen (N₂) purge for 15 minutes. Cu(ᵢ)Cl (42.8 mg, 0.4318 mmol, 1 eq.) was added to the flask and left to purge for a further 5 minutes. After the conversion of DEAEMA reached around 85%, the mixture from the second flask was added into the first flask rapidly using a syringe and taking care not to admit any air into the vessel. A sample was taken immediately after the addition of the tBuMA monomer solution for ¹H NMR analysis. The block copolymerization reaction was carried out at ambient temperature and samples were taken periodically from the reaction mixture for ¹H NMR analysis. Reactions were terminated when >99% conversion was reached, as judged by ¹H NMR, by exposure to oxygen and addition of acetone. The catalyst residues were removed by passing the mixture over a basic alumina column. Acetone was removed under vacuum to concentrate the sample before precipitation into cold petroleum ether (40° C.-60° C.) and drying in the vacuum oven overnight.

| | Mw/Da | Mn/Da | PDI | Z-Ave/nm | PdI |
|---|---|---|---|---|---|
| DEAEMA₅₀ | 374,192 | 129,737 | 2.9 | 162.9 | 0.082 |
| tBuMA₆₅- | | | | | |
| EGDMA_{0.9} | | | | | |

### 11. Nanoparticle Formation in Respect of Polydendrons of Sections 8 to 10

In a typical procedure, 10 mg of sample was completely dissolved in 2 mL of acetone at room temperature; the resulting solution (5 mg mL⁻¹) was added drop wise to 10 mL of distilled water under vigorous stirring for ca. 15 min using a glass pipette. The solution was stirred vigorously for 24 h at room temperature, until the acetone was completely evaporated as determined by ¹H NMR analysis, where no peak at δ 2.22 corresponding to acetone was observed.

### 12. Nile Red Encapsulation in Respect of Polydendrons of Sections 8 to 10

In a typical procedure, 10 mg of sample and 0.1 mg Nile Red was dissolved completely in 2 mL of acetone at room temperature; the resulting solution (5.05 mg mL⁻¹) was added drop wise to 10 mL of distilled water under vigorous stirring for ca. 15 min using a glass pipette. The solution was stirred vigorously for 24 h at room temperature, until the acetone was completely evaporated as determined by ¹H NMR analysis, where no peak at δ 2.22 corresponding to acetone was observed.

### 13. Fluorescenamine Encapsulation in Respect of Polydendrons of Sections 8 to 10

In a typical procedure, 10 mg of sample and 1 mg of Fluoresceinamine was dissolved completely in 2 mL of acetone at room temperature; the resulting solution (5.5 mg mL⁻¹) was added drop wise to 10 mL of distilled water under vigorous stirring for ca. 15 min using a glass pipette. The solution was stirred vigorously for 24 h at room temperature, until the acetone was completely evaporated as determined by ¹H NMR analysis, where no peak at δ 2.22 corresponding to acetone was observed.

### 14. Example of Nanoprecipitation to Encapsulate Inorganic Magnetic Nanoparticles

Polydendron (G2:2K PEG(50:50)-pHPMA₅₀-EGDMA_{0.8}) was dissolved in THF for a minimum of 6 hours. Once fully dissolved the polymer in THF (0.2 ml, 25 mg/ml) was mixed with Fe₃O₄ 10 nm particles in THF (0.5 ml, 5 mg/ml) and this mixture of polymer and Fe₃O₄ was added quickly to a vial of water (1 ml) stirring at 30° C. The solvent was allowed to evaporate overnight in a fume cupboard to give a final concentration of 5 mg/ml polymer, 2.5 mg/ml Fe₃O₄ in water. The nanoparticles formed were analysed by dynamic light scattering (DLS), scanning electron microscopy (SEM) and transmission electron microscopy (TEM).

SEM imaging showed spherical nanoparticles of size range varying from approximately 150 to 250 nm while TEM imaging showed the majority of nanoparticles to have encapsulated Fe₃O₄ with no free Fe₃O₄ observed.

DLS (2.5 mg/ml in water) determined the Z-Ave hydrodynamic diameter to be 182 nm with PDI to be 0.01. In the presence of a magnetic field (i.e. with a magnetic suspended above, just touching the surface of the dispersion) DLS measurements showed a 50% reduction in derived count rate after 12 hours and a 40% reduction in derived count rate after 8 hours, with Z-Ave diameter remaining constant throughout. The reduction in derived count rate is intrinsic to a decrease in concentration of nanoparticles within the dispersion and demonstrates the effect of the magnetic field on directing the behaviour of the nanoprecipitate. In the absence of a magnetic field there is no drop in derived count rate.

### 15. Illustrations of Some Effects of the Polydendrons Including pH Responsive Effects

FIGS. 15 to 19 illustrate some effects of the polydendrons including pH responsive effects.

FIG. 15 is a photograph showing the encapsulation of oil red into amine containing polydendron nanoprecipitates. In the vial on the left, oil red is not dissolved in water due to the inherent hydrophobicity of the dye (in the original photograph, the fluid in the vial is almost colourless). In the vial on the right, oil red is encapsulated in polydendron nanoprecipitate (in the original photograph, the fluid in the vial is dark red).

FIG. 16 contains photographs showing fluoresceinamine encapsulated within poly(DEAEMA) polydendron in a dialysis bag. The photograph on the left shows the hydrophobic dye encapsulated in the polydendron nanoprecipitate after standing in an aqueous solution at neutral pH for 24 hours (in the original photograph, a yellow colour is confined to the dialysis bag). The photograph on the right shows the release of the dye into the dialysis sink water after addition of HCl to the sink water thereby triggering release from the polydendron nanoprecipitates (in the original photograph, a yellow colour is visible throughout the fluid in the beaker, not just confined to the dialysis bag).

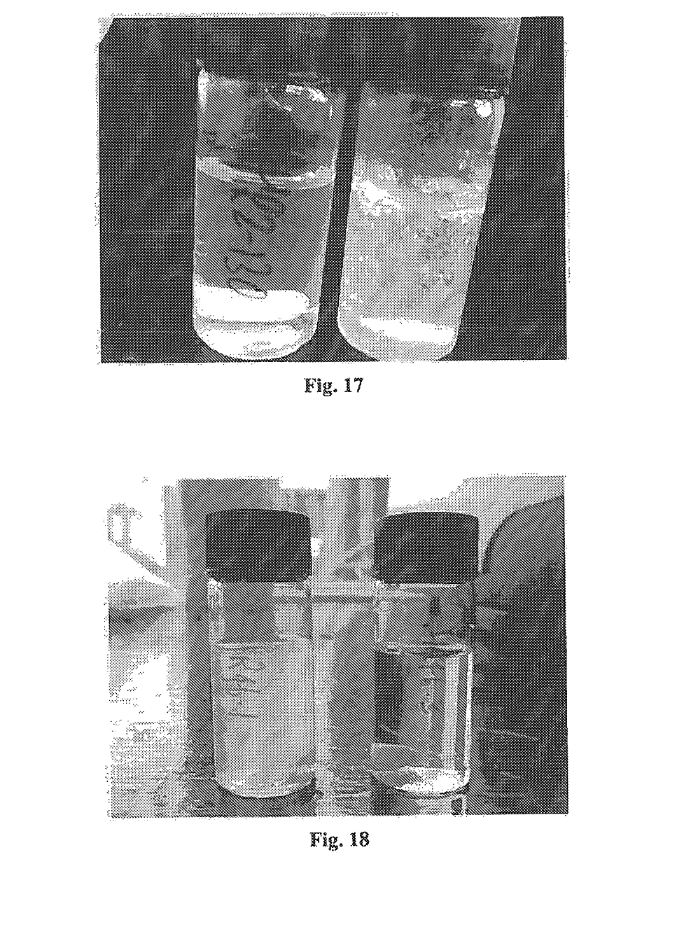

FIG. 17 shows a photograph of two vials. The vial on the left contains an amine containing dendron initiated polydendron nanoprecipiate in water after the addition of transport buffer. The vial on the right shows a branched polymer nanoprecipitate (without amine containing dendron end groups) after the addition of transport buffer. This shows that the presence of the dendron prevents precipitation.

FIG. 18 is a photograph showing nanoprecipitated amine containing polydendron at neutral pH (left) and after addition of HCl (right). The clarity of the vial on the right (compared to the cloudiness of the vial of the left) indicates solvation and lack of nanoprecipitated particles after HCl addition.

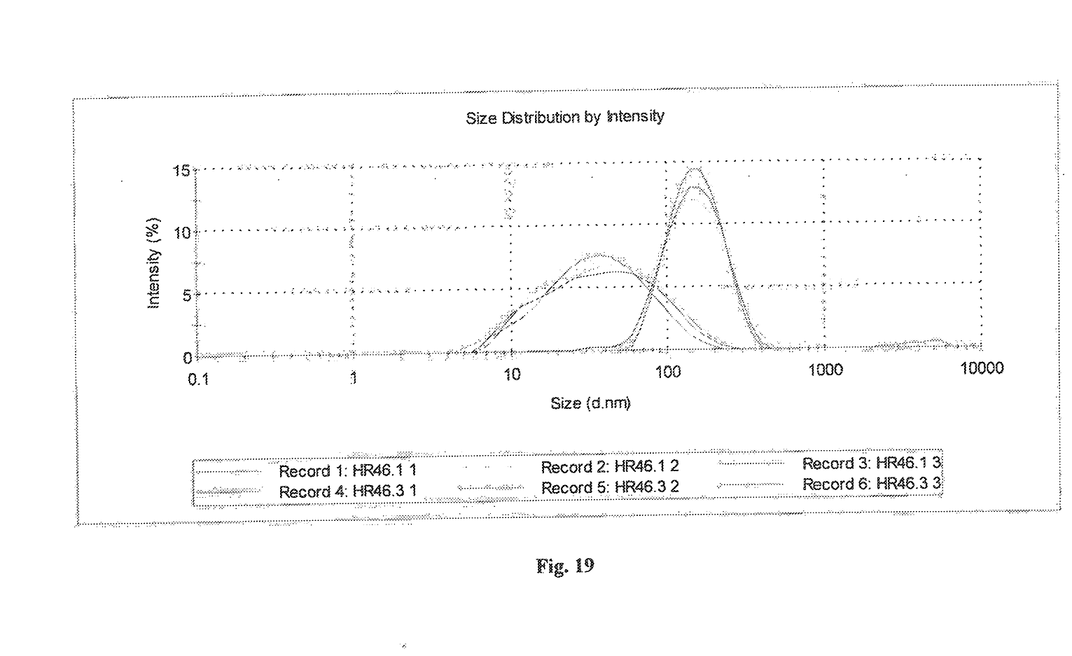

FIG. 19 shows a dynamic light scattering (DLS) trace from amine containing polydendron nanoprecipiates at neutral pH (sharp peak to the right: approx z-average 136 nm, PDI 0.14) and the same sample after addition of HCl (broad peak to the left: approx z-average 28 nm, PDI 0.38). The change in particle size and increased PDI shows solvation and disassembly of the nanoprecipitate.

1. A method of preparing a pH-responsive non-gelled branched vinyl polymer scaffold carrying dendrons, comprising the living or controlled polymerization of a monofunctional vinyl monomer and a difunctional vinyl monomer, using a dendron initiator.

2. The method as claimed in claim 1 wherein the living polymerization is ATRP.

3. The method as claimed in claim 1 wherein the molar ratio of difunctional vinyl monomer to initiators is less than 1.

4. The method as claimed in claim 1 wherein a further initiator is used selected from or comprising one or more of the following: a small molecule, a drug, an active pharmaceutical ingredient, a polymer, a peptide, a sugar, a dendron, a moiety which carries or can carry a drug, an anionic functional group, a cationic functional group, a moiety which enhances solubility, a moiety which prolongs residence time within the body, a moiety which enhances stability of a drug or other active material, a moiety which reduces macrophage uptake, a moiety which enhances controlled release, a moiety which enhances drug transport, or a moiety which enhances drug targeting.

5. The method as claimed in claim 1 wherein the further initiator comprises a PEG group.

6. The method as claimed in claim 1 wherein the dendron initiator comprises a generation 1 dendron.

7. The method as claimed in claim 6 wherein the first generation branches are identical.

8. The method as claimed in claim 1 wherein the dendron initiator comprises a generation 2 dendron.

9. The method as claimed in claim 8 wherein the second generation branches are identical.

10. The method as claimed in claim 1 wherein one or more of the initiators comprises a functional group allowing post-functionalization.

11. The method as claimed in claim 1 followed by nanoprecipitation to form nanoparticles.

12. The method as claimed in claim 1 wherein the monofunctional vinyl monomer and/or the difunctional vinyl monomer comprise a methacrylate.

13. A product obtained by the method of claim 1.

14. A pH responsive non-gelled branched vinyl polymer scaffold carrying a dendron moiety.

15. The scaffold as claimed in claim 14 which is an atom transfer radical polymerized material.

16. The scaffold as claimed in claim 14 carrying a further moiety selected from one or more of the following: a small molecule, a drug, an active pharmaceutical ingredient, a polymer, a peptide, a sugar, a dendron, a moiety which carries or can carry a drug, an anionic functional group, a cationic functional group, a moiety which enhances solubility, a moiety which prolongs residence time within the body, a moiety which enhances stability of a drug or other active material, a moiety which reduces macrophage uptake, a moiety which enhances controlled release, a moiety which enhances drug transport, or a moiety which enhances drug targeting.

17. The scaffold as claimed in claim 14 wherein the further moiety comprises a PEG group.

18. The scaffold as claimed in claim 14 wherein the dendron initiator comprises a generation 1 dendron.

19. The scaffold as claimed in claim 18 wherein the first generation branches are identical.

20. The scaffold as claimed in claim 14 wherein the dendron initiator comprises a generation 2 dendron.

21. The scaffold as claimed in claim 20 wherein the second generation branches are identical.

22. The scaffold as claimed in claim 14 wherein one or more of the initiators comprises a functional group allowing post-functionalization.

23. A nanoparticle comprising the scaffold as claimed in claim 14.

24. A pharmaceutical composition comprising the product as claimed in claim 13 and a pharmaceutically acceptable diluent.

25. The pharmaceutical composition as claimed in claim 24 which is formulated for oral, parenteral, topical or ocular administration.

26-30. (canceled)

31. A method of treatment comprising administration of the product as claimed in claim 13 to a patient in need thereof.

32. A method of releasing an encapsulated or carried material from a product as claimed in claim 13 comprising altering the pH of the environment.

33. The method as claimed in claim 32 wherein the encapsulated or carried material is, or releases, a drug.
